SEQUENCE LISTING (21 sequences)

SEQ ID NO: 1 - RNA, 2341 nt, homo_sapiens
gggaggccggaugugaguggagcggccauuuccuguuucucugcaguuuuccucagcuuugggugguggccgcugccgggcaucggcuuccaguccgcggagggcgaggcggcguggacagcggccccggcacccagcgccccgccgcccgcaagccgcgcgcccguccgccgcgccccgagcccgccgcuuccuaucucagcgcccugccgccgccgccgcggcccagcgagcggcccugaugcaggccaucaagugugugguggugggagacggagcuguagguaaaacuugccuacugaucaguuacacaaccaaugcauuuccuggagaauauaucccuacugucuuugacaauuauucugccaauguuaugguagauggaaaaccggugaaucugggcuuaugggauacagcuggacaagaagauuaugacagauuacgcccccuauccuauccgcaaacagauguguucuuaauuugcuuuucccuugugaguccugcaucauuugaaaauguccgugcaaagugguauccugaggugcggcaccacugucccaacacucccaucauccuagugggaacuaaacuugaucuuagggaugauaaagacacgaucgagaaacugaaggagaagaagcugacucccaucaccuauccgcagggucuagccauggcuaaggagauuggugcuguaaaauaccuggagugcucggcgcucacacagcgaggccucaagacaguguuugacgaagcgauccgagcaguccucugcccgccucccgugaagaagaggaagagaaaaugccugcuguuguaaaugucucagccccucguucuugguccugucccuuggaaccuuuguacgcuuugcucaaaaaaaaacaaaaaaaaaaaacaaaaaaaaaaaacaacgguggagccuucgcacucaaugccaacuuuuuguuacagauuaauuuuuccauaaaaccauuuuuugaaccaaucaguaauuuuaagguuuuguuuguucuaaauguaagaguucagacucacauucuauuaaaauuuagcccuaaaaugacaagccuucuuaaagccuuauuuuucaaaagcgccccccccauucuuguucagauuaagaguugccaaaauaccuucugaacuacacugcauuguugugccgagaacaccgagcacugaacuuugcaaagaccuucgucuuugagaagacgguagcuucugcaguuaggaggugcagacacuugcucuccuauguaguucucagaugcguaaagcagaacagccucccgaaugaagcguugccauugaacucaccagugaguuagcagcacguguucccgacauaacauuguacuguaauggagugagcguagcagcucagcucuuuggaucagucuuugugauuucauagcgaguuuucugaccagcuuuugcggagauuuugaacagaacugcuauuuccucuaaugaagaauucuguuuagcugugggugugccggguggggugugugugaucaaaggacaaagacaguauuuugacaaaauacgaaguggagauuuacacuacauuguacaaggaaugaaagugucacggguaaaaacucuaaaagguuaauuucugucaaaugcaguagaugaugaaagaaagguugguauuaucaggaaauguuuucuuaagcuuuuccuuucucuuacaccugccaugccuccccaaauugggcauuuaauucaucuuuaaacugguuguucuguuagucgcuaacuuaguaagugcuuuucuuauagaaccccuucugacugagcaauaugccuccuuguauuauaaaaucuuucugauaaugcauuagaagguuuuuuugucgauuaguaaaagugcuuuccauguuacuuuauucagagcuaauaagugcuuuccuuaguuuucuaguaacuagguguaaaaaucauguguugcagcuuuauaguuuuuaaaauauuuuagauaauucuuaaacuaugaaccuucuuaacaucacugucuugccagauuaccgacacugucacuugaccaauacugacccucuuuaccucgcccacgcggacacacgccuccuguagucgcuuugccuauugauguuccuuugggucugugagguucuguaaacugugcuagugcugacgauguucuguacaacuuaacucacuggcgagaauacagcgugggacccuucagccacuacaacagaauuuuuuaaauugacaguugcagaauuguggaguguuuuuacauugaucuuuugcuaaugcaauuagcauuauguuuugcauguaugacuuaauaaauccuugaaucaua

SEQ ID NO: 2 - RNA, 1319 nt, homo_sapiens
cgcgcuuggccuugcccgcgcccgcucgccucgucucgcccggccuccccgcgucgccucgucgccuguuccgcgccaggcauggcccccagcacuguggccguggagcugcucagccccaaagagaaaaaccgacugcggaagccggugguggagaagaugcgccgcgaccgcaucaacagcagcaucgagcagcugaagcugcugcuggagcaggaguucgcgcggcaccagcccaacuccaagcuggagaaggccgacauccuggagauggcugucagcuaccugaagcacagcaaagccuucgucgccgccgccggccccaagagccugcaccaggacuacagcgaaggcuacucguggugccugcaggaggccgugcaguuccugacgcuccacgccgccagcgacacgcagaugaagcugcuguaccacuuccagcggcccccggccgcgcccgccgcgcccgccaaggagcccaaggcgccgggcgccgcgcccccgcccgcgcucuccgccaaggccaccgccgccgccgccgccgcgcaccagcccgccugcggccucuggcggcccuggugacccggcgggaccugcgggcgcgcggcccgacgaccagagggcgagccugcuccucucgccuguagggaagcgccuucccgccgucguccgccccgggcuuggacgcgcccuucuccggaaggcucuggccccaagcuggccggcccgcaggagccccauucucagagaaugugugugcagagucccugccguuuuaggacaaucagggcccaucuucugccaagugucugaccccaugggguuguucuguguuugcauuuaagcaagugacuucugggaaguccccggccgcccgggguucuaugauauuuguagugccggggcucgcacacugcugcccccagccuguagaggacuuucuucagggcccguagcugcugggcguaccccuggcaggcgggcugugccgcgggcacauuugccuuuugugaaggccgaacucgagcuguauccucauaggaaacagugaucaccccggacgggcguccaggacccugagggccauggccaaaaggcuccugagugugccugguggucuggcuggggcucacggugggcugucuggggagggugggugccuccacuaugauccuuaaaggauuccucuguguggguggaugcgugugggcacgacuuuguacucagaaauugaacucucagucacguggaagccacgggacugcuccgaagccgccauaauaaaaucugauuguucagcccccaaaaaaaaaaaaaaa

SEQ ID NO: 3 - RNA, 2672 nt, homo_sapiens
gcguggccggcgccggcucuugcggccgagcagaguugcggcgugggaaagagccgcuaggagcagaccgcgccgccgccggagccgcgccugcccaggcccggggagggaggaggcgggcgucagggugcugcgccccgcucggcguccgagcuuccggccgggcugugccccgcgcggucuucgccgggaugaagcgccccugcgaggagacgaccuccgagagcgacauggacgagaccaucgacguggggagcgagaacaauuacucggggcaaaguacuagcucugugauuagauugaauucuccaacaacaacaucucagauuauggcaagaaagaaaaggagagggauuauagagaaaaggcgucgggaucggauaaauaacaguuuaucugaguugagaagacuugugccaacugcuuuugaaaaacaaggaucugcaaaguuagaaaaagcugaaauauugcaaaugacaguggaucauuugaagaugcuucaggcaacaggggguaaaggcuacuuugacgcacacgcucuugccauggacuucaugagcauaggauuccgagagugccuaacagaaguugcgcgguaccugagcuccguggaaggccuggacuccucggauccgcugcgggugcggcuugugucucaucucagcacuugcgccacccagcgggaggcggcggccaugacauccuccauggcccaccaccaucauccgcuccacccgcaucacugggccgccgccuuccaccaccugcccgcagcccugcuccagcccaacggccuccaugccucagagucaaccccuugucgccucuccacaacuucagaagugccuccugcccacggcucugcucuccucacggccacguuugcccaugcggauucagcccuccgaaugccauccacgggcagcgucgcccccugcgugccaccucucuccaccucucucuugucccucucugccaccguccacgccgcagccgcagcagccaccgcggcugcacacagcuucccucuguccuucgcgggggcauuccccaugcuucccccaaacgcagcagcagcaguggccgcggccacagccaucagcccgcccuugucaguaucagccacguccaguccucagcagaccagcaguggaacaaacaauaaaccuuaccgacccugggggacagaaguuggagcuuuuuaaauuuuucuugaacuucuugcaauaguaacugaauguccuccauuucagagucagcuuaaaaccucugcacccugaagguagccauacagaugccgacagauccacaaaggaacaauaaagcuauuugagacacaaaccucacgaguggaaaugugguauucucuuuuuuuucucucccuuuuuuguuugguucaaggcagcucgguaacugacaucagcaacuuuugaaaacuucacacuuguuaccauuuagaaguuuccuggaaaauauauggaccguaccauccagcagugcaucaguaugucugaauuggggaaguaaaaugcccugacugaauucucuugagacuagaugggacauacauauauagagagagagugagagagucguguuucguaagugccugagcuuaggaaguuuucuucuggauauauaacauugcacaagggaagacgaguguggaggauagguuaagaaaggaaagggacagaagucuugcaauaggcugcagacauuuuaauaccaugccagagaagaguauucugcugaaaccaacagguuuuacuggucaaaaugacugcugaaaauaauuuucaaguugaaagaucuaguuuuaucuuaguuugccuucuuuguacagacaugccaagaggugacauuuagcagugcauugguauaagcaauuauuucaucaguucucagauuaacaagcauuucugcucugccugcaggcccccaggcacuuuuuuuuuuggauggcucaaaauauggugcugcuuuauauaaaccuuacauuuauauagugcaccuaugagcaguugccuaccauguguccaccagaggcuauuuaauucaugccaacuugaaaacucuccaguuuguaggaguuugguuuaauuuauucaguuucauuaggacuauuuuuauauauuuauccucuucauuuucuccuaaugaugcaacaucuauucuugucacccuuugggagaaguuacauuucuggaggugaugaagcaaggagggagcacuaggaagagaaaagcuacaauuuuuaaagcucuuugucaaguuagugauugcauuugaucccaaaacaagaugaauguaugcaaugggauguacauaaguuauuuuugcccaugccuaaacuagugcuauguaaugggguugugguuuuguuuuuuucgauuucguuuaaugacaaaauaaucucuuaauaugcugaaaucaagcacgugagaguuuuuguuuaaaagauaagagacacagcauguauuaugcacuucauuucucuacuguguggagaaagcaauaaacauuaugagaauguuaaacguuaugcaaaauuauacuuuuaaauauuuguuuugaaauuacuguaccuagucuuuuuugcauuacuuuguaaccuuuuucuaugcaagagucuuuacauaccacuaauuaaaugaaguccuuuuugacua

SEQ ID NO: 4 - RNA, 2862 nt, homo_sapiens
agauuccuacuucuuacgccccccacaucacccgccucgagaccucaaggguagaggugggcacccccgccuccgcacuuuugcucggggcuccagauuguagggcagggcggcgcuucucggaaagcgaaagccggcggggcggggcgggugccgcaggagaaagaggaagcgcuggcagacaaugcgacccgaccgcgcugaggcuccaggaccgcccgccauggcugcaggaggucccggcgcggggucugcggccccggucuccuccacauccucccuuccccuggcugcucucaacaugcgagugcggcgccgccugucucuguucuugaacgugcggacacagguggcggccgacuggaccgcgcuggcggaggagauggacuuugaguacuuggagauccggcaacuggagacacaagcggaccccacuggcaggcugcuggacgccuggcagggacgcccuggcgccucuguaggccgacugcucgagcugcuuaccaagcugggccgcgacgacgugcugcuggagcugggacccagcauugaggaggauugccaaaaguauaucuugaagcagcagcaggaggaggcugagaagccuuuacagguggccgcuguagacagcagugucccacggacagcagagcuggcgggcaucaccacacuugaugacccccuggggcauaugccugagcguuucgaugccuucaucugcuauugccccagcgacauccaguuugugcaggagaugauccggcaacuggaacagacaaacuaucgacugaaguugugugugucugaccgcgauguccugccuggcaccugugucuggucuauugcuagugagcucaucgaaaagaggugccgccggaugguggugguugucucugaugauuaccugcagagcaaggaaugugacuuccagaccaaauuugcacucagccucucuccaggugcccaucagaagcgacugauccccaucaaguacaaggcaaugaagaaagaguuccccagcauccugagguucaucacugucugcgacuacaccaaccccugcaccaaaucuugguucuggacucgccuugccaaggccuugucccugcccugaagacuguucugaggcccuggguguguguguaucugucugccuguccauguacuucugcccugccuccuccuuucguuguaggaggaaucugugcucuacuuaccucucaauuccuggagaugccaacuucacagacacgucugcagcagcuggacaucacauuucauguccugcauggaaccaguggcugugaguggcauguccacuugcuggauuaucagccaggacacuauagaacaggaccagcugagacuaagaaggaccagcagagccagcucagcucugagccauucacacaucuucacccucaguuuccucacuugaggagugggauggggagaacagagaguagcuguguuugaaucccuguaggaaauggugaagcauagcucugggucuccugggggagaccaggcuuggcugcgggagagcuggcuguugcuggacuacaugcuggccacugcugugaccacgacacugcuggggcagcuucuuccacagugaugccuacugaugcuucagugccucugcacaccgcccauuccacuuccuccuuccccacagggcagguggggaagcaguuuggcccagcccaaggagaccccaccuugagccuuauuuccuaauggguccaccucucaucugcaucuuucacaccucccagcuucugcccaaccuucagcagugacaaguccccaagagacucgccugagcagcuugggcugcuuuucauuuccaccugucaggaugccuguggucaugcucucagcuccaccuggcaugagaagggauccuggccucuggcauauucaucaaguaugaguucuggggaugagucacuguaaugaugugagcagggagccuuccucccugggccaccugcagagagcuuucccaccaacuuuguaccuugauugccuuacaaaguuauuuguuuacaaacagcgaccauauaaaagccuccugccccaaagcuugugggcacaugggcacauacagacucacauacagacacacacauauauguacagacauguacucucacacacacaggcaccagcauacacacguuuuucuagguacagcucccaggaacagcuaggugggaaagucccaucacugagggagccuaaccaugucccugaacaaaaauugggcacucaucuauuccuuuucucuugugucccuacucauugaaaccaaacucuggaaaggacccaauguaccaguauuuauaccucuaaugaagcacagagagaggaagagagcugcuuaaacucacacaacaaugaacugcagacacagcuguucucucccucucuccuucccagagcaauuuauacuuuacccucaggcuguccucuggggagaaggugccauggucuuaggugucugugccccaggacagacccuaggacccuaaauccaauagaaaaugcauaucuuugcuccacuuucagccaggcuggagcaagguaccuuuucuuaggaucuugggagggaauggaugccccucucugcaugaucuuguugaggcauuuagcugccaugcaccugucccccuuuaauacugggcauuuuaaagccaucucaagaggcaucuucuacauguuuuguacgcauuaaaauaauuucaaagauaucugagaaaagccgauauuugccauucuuccuauauccuggaauauaucuugcauccugaguuuauaauaauaaauaauauucuaccuuggaaaaaaaaaaaaaaa

SEQ ID NO: 5 - RNA, 2681 nt, homo_sapiens
agauuccuacuucuuacgccccccacaucacccgccucgagaccucaaggguagaggugggcacccccgccuccgcacuuuugcucggggcuccagauuguagggcagggcggcgcuucucggaaagcgaaagccggcggggcggggcgggugccgcaggagaaagaggaagcgcuggcagacaaugcgacccgaccgcgcugaggcuccaggaccgcccgccauggcugcaggaggucccggcgcggggucugcggccccggucuccuccacauccucccuuccccuggcugcucucaacaugcgagugcggcgccgccugucucuguucuugaacgugcggacacagguggcggccgacuggaccgcgcuggcggaggagauggacuuugaguacuuggagauccggcaacuggagacacaagcggaccccacuggcaggcugcuggacgccuggcagggacgcccuggcgccucuguaggccgacugcucgagcugcuuaccaagcugggccgcgacgacgugcugcuggagcugggacccagcauugaggaggauugccaaaaguauaucuugaagcagcagcaggaggaggcugagaagccuuuacagguggccgcuguagacagcagugucccacggacagcagagcuggcgggcaucaccacacuugaugacccccugggugccgccggaugguggugguugucucugaugauuaccugcagagcaaggaaugugacuuccagaccaaauuugcacucagccucucuccaggugcccaucagaagcgacugauccccaucaaguacaaggcaaugaagaaagaguuccccagcauccugagguucaucacugucugcgacuacaccaaccccugcaccaaaucuugguucuggacucgccuugccaaggccuugucccugcccugaagacuguucugaggcccuggguguguguguaucugucugccuguccauguacuucugcccugccuccuccuuucguuguaggaggaaucugugcucuacuuaccucucaauuccuggagaugccaacuucacagacacgucugcagcagcuggacaucacauuucauguccugcauggaaccaguggcugugaguggcauguccacuugcuggauuaucagccaggacacuauagaacaggaccagcugagacuaagaaggaccagcagagccagcucagcucugagccauucacacaucuucacccucaguuuccucacuugaggagugggauggggagaacagagaguagcuguguuugaaucccuguaggaaauggugaagcauagcucugggucuccugggggagaccaggcuuggcugcgggagagcuggcuguugcuggacuacaugcuggccacugcugugaccacgacacugcuggggcagcuucuuccacagugaugccuacugaugcuucagugccucugcacaccgcccauuccacuuccuccuuccccacagggcagguggggaagcaguuuggcccagcccaaggagaccccaccuugagccuuauuuccuaauggguccaccucucaucugcaucuuucacaccucccagcuucugcccaaccuucagcagugacaaguccccaagagacucgccugagcagcuugggcugcuuuucauuuccaccugucaggaugccuguggucaugcucucagcuccaccuggcaugagaagggauccuggccucuggcauauucaucaaguaugaguucuggggaugagucacuguaaugaugugagcagggagccuuccucccugggccaccugcagagagcuuucccaccaacuuuguaccuugauugccuuacaaaguuauuuguuuacaaacagcgaccauauaaaagccuccugccccaaagcuugugggcacaugggcacauacagacucacauacagacacacacauauauguacagacauguacucucacacacacaggcaccagcauacacacguuuuucuagguacagcucccaggaacagcuaggugggaaagucccaucacugagggagccuaaccaugucccugaacaaaaauugggcacucaucuauuccuuuucucuugugucccuacucauugaaaccaaacucuggaaaggacccaauguaccaguauuuauaccucuaaugaagcacagagagaggaagagagcugcuuaaacucacacaacaaugaacugcagacacagcuguucucucccucucuccuucccagagcaauuuauacuuuacccucaggcuguccucuggggagaaggugccauggucuuaggugucugugccccaggacagacccuaggacccuaaauccaauagaaaaugcauaucuuugcuccacuuucagccaggcuggagcaagguaccuuuucuuaggaucuugggagggaauggaugccccucucugcaugaucuuguugaggcauuuagcugccaugcaccugucccccuuuaauacugggcauuuuaaagccaucucaagaggcaucuucuacauguuuuguacgcauuaaaauaauuucaaagauaucugagaaaagccgauauuugccauucuuccuauauccuggaauauaucuugcauccugaguuuauaauaauaaauaauauucuaccuuggaaaaaaaaaaaaaaa

SEQ ID NO: 6 - RNA, 2886 nt, homo_sapiens
agauuccuacuucuuacgccccccacaucacccgccucgagaccucaaggguagaggugggcacccccgccuccgcacuuuugcucggggcuccagauuguagggcagggcggcgcuucucggaaagcgaaagccggcggggcggggcgggugccgcaggagaaagaggaagcgcuggcagacaaugcgacccgaccgcgcugaggcuccaggaccgcccgccauggcugcaggaggucccggcgcggggucugcggccccggucuccuccacauccucccuuccccuggcugcucucaacaugcgagugcggcgccgccugucucuguucuugaacgugcggacacagguggcggccgacuggaccgcgcuggcggaggagauggacuuugaguacuuggagauccggcaacuggagacacaagcggaccccacuggcaggcugcuggacgccuggcagggacgcccuggcgccucuguaggccgacugcucgagcugcuuaccaagcugggccgcgacgacgugcugcuggagcugggacccagcauugaggaggauugccaaaaguauaucuugaagcagcagcaggaggaggcugagaagccuuuacagguggccgcuguagacagcagugucccacggacagcagagcuggcgggcaucaccacacuugaugacccccuggggcauaugccugagcguuucgaugccuucaucugcuauugccccagcgacauccaguuugugcaggagaugauccggcaacuggaacagacaaacuaucgacugaaguugugugugucugaccgcgauguccugccuggcaccugugucuggucuauugcuagugagcucaucgaaaagagguuggcuagaaggccacggggugggugccgccggaugguggugguugucucugaugauuaccugcagagcaaggaaugugacuuccagaccaaauuugcacucagccucucuccaggugcccaucagaagcgacugauccccaucaaguacaaggcaaugaagaaagaguuccccagcauccugagguucaucacugucugcgacuacaccaaccccugcaccaaaucuugguucuggacucgccuugccaaggccuugucccugcccugaagacuguucugaggcccuggguguguguguaucugucugccuguccauguacuucugcccugccuccuccuuucguuguaggaggaaucugugcucuacuuaccucucaauuccuggagaugccaacuucacagacacgucugcagcagcuggacaucacauuucauguccugcauggaaccaguggcugugaguggcauguccacuugcuggauuaucagccaggacacuauagaacaggaccagcugagacuaagaaggaccagcagagccagcucagcucugagccauucacacaucuucacccucaguuuccucacuugaggagugggauggggagaacagagaguagcuguguuugaaucccuguaggaaauggugaagcauagcucugggucuccugggggagaccaggcuuggcugcgggagagcuggcuguugcuggacuacaugcuggccacugcugugaccacgacacugcuggggcagcuucuuccacagugaugccuacugaugcuucagugccucugcacaccgcccauuccacuuccuccuuccccacagggcagguggggaagcaguuuggcccagcccaaggagaccccaccuugagccuuauuuccuaauggguccaccucucaucugcaucuuucacaccucccagcuucugcccaaccuucagcagugacaaguccccaagagacucgccugagcagcuugggcugcuuuucauuuccaccugucaggaugccuguggucaugcucucagcuccaccuggcaugagaagggauccuggccucuggcauauucaucaaguaugaguucuggggaugagucacuguaaugaugugagcagggagccuuccucccugggccaccugcagagagcuuucccaccaacuuuguaccuugauugccuuacaaaguuauuuguuuacaaacagcgaccauauaaaagccuccugccccaaagcuugugggcacaugggcacauacagacucacauacagacacacacauauauguacagacauguacucucacacacacaggcaccagcauacacacguuuuucuagguacagcucccaggaacagcuaggugggaaagucccaucacugagggagccuaaccaugucccugaacaaaaauugggcacucaucuauuccuuuucucuugugucccuacucauugaaaccaaacucuggaaaggacccaauguaccaguauuuauaccucuaaugaagcacagagagaggaagagagcugcuuaaacucacacaacaaugaacugcagacacagcuguucucucccucucuccuucccagagcaauuuauacuuuacccucaggcuguccucuggggagaaggugccauggucuuaggugucugugccccaggacagacccuaggacccuaaauccaauagaaaaugcauaucuuugcuccacuuucagccaggcuggagcaagguaccuuuucuuaggaucuugggagggaauggaugccccucucugcaugaucuuguugaggcauuuagcugccaugcaccugucccccuuuaauacugggcauuuuaaagccaucucaagaggcaucuucuacauguuuuguacgcauuaaaauaauuucaaagauaucugagaaaagccgauauuugccauucuuccuauauccuggaauauaucuugcauccugaguuuauaauaauaaauaauauucuaccuuggaaaaaaaaaaaaaaa

SEQ ID NO: 7 - RNA, 2546 nt, homo_sapiens
agauuccuacuucuuacgccccccacaucacccgccucgagaccucaaggguagaggugggcacccccgccuccgcacuuuugcucggggcuccagauuguagggcagggcggcgcuucucggaaagcgaaagccggcggggcggggcgggugccgcaggagaaagaggaagcgcuggcagacaaugcgacccgaccgcgcugaggcuccaggaccgcccgccauggcugcaggaggucccggcgcggggucugcggccccggucuccuccacauccucccuuccccuggcugcucucaacaugcgagugcggcgccgccugucucuguucuugaacgugcggacacagguggcggccgacuggaccgcgcuggcggaggagauggacuuugaguacuuggagauccggcaacuggagacacaagcggaccccacuggcaggcugcuggacgccuggcagggacgcccuggcgccucuguaggccgacugcucgagcugcuuaccaagcugggccgcgacgacgugcugcuggagcugggacccagcauuggugccgccggaugguggugguugucucugaugauuaccugcagagcaaggaaugugacuuccagaccaaauuugcacucagccucucuccaggugcccaucagaagcgacugauccccaucaaguacaaggcaaugaagaaagaguuccccagcauccugagguucaucacugucugcgacuacaccaaccccugcaccaaaucuugguucuggacucgccuugccaaggccuugucccugcccugaagacuguucugaggcccuggguguguguguaucugucugccuguccauguacuucugcccugccuccuccuuucguuguaggaggaaucugugcucuacuuaccucucaauuccuggagaugccaacuucacagacacgucugcagcagcuggacaucacauuucauguccugcauggaaccaguggcugugaguggcauguccacuugcuggauuaucagccaggacacuauagaacaggaccagcugagacuaagaaggaccagcagagccagcucagcucugagccauucacacaucuucacccucaguuuccucacuugaggagugggauggggagaacagagaguagcuguguuugaaucccuguaggaaauggugaagcauagcucugggucuccugggggagaccaggcuuggcugcgggagagcuggcuguugcuggacuacaugcuggccacugcugugaccacgacacugcuggggcagcuucuuccacagugaugccuacugaugcuucagugccucugcacaccgcccauuccacuuccuccuuccccacagggcagguggggaagcaguuuggcccagcccaaggagaccccaccuugagccuuauuuccuaauggguccaccucucaucugcaucuuucacaccucccagcuucugcccaaccuucagcagugacaaguccccaagagacucgccugagcagcuugggcugcuuuucauuuccaccugucaggaugccuguggucaugcucucagcuccaccuggcaugagaagggauccuggccucuggcauauucaucaaguaugaguucuggggaugagucacuguaaugaugugagcagggagccuuccucccugggccaccugcagagagcuuucccaccaacuuuguaccuugauugccuuacaaaguuauuuguuuacaaacagcgaccauauaaaagccuccugccccaaagcuugugggcacaugggcacauacagacucacauacagacacacacauauauguacagacauguacucucacacacacaggcaccagcauacacacguuuuucuagguacagcucccaggaacagcuaggugggaaagucccaucacugagggagccuaaccaugucccugaacaaaaauugggcacucaucuauuccuuuucucuugugucccuacucauugaaaccaaacucuggaaaggacccaauguaccaguauuuauaccucuaaugaagcacagagagaggaagagagcugcuuaaacucacacaacaaugaacugcagacacagcuguucucucccucucuccuucccagagcaauuuauacuuuacccucaggcuguccucuggggagaaggugccauggucuuaggugucugugccccaggacagacccuaggacccuaaauccaauagaaaaugcauaucuuugcuccacuuucagccaggcuggagcaagguaccuuuucuuaggaucuugggagggaauggaugccccucucugcaugaucuuguugaggcauuuagcugccaugcaccugucccccuuuaauacugggcauuuuaaagccaucucaagaggcaucuucuacauguuuuguacgcauuaaaauaauuucaaagauaucugagaaaagccgauauuugccauucuuccuauauccuggaauauaucuugcauccugaguuuauaauaauaaauaauauucuaccuuggaaaaaaaaaaaaaaa

SEQ ID NO: 8 - RNA, 2727 nt, homo_sapiens
agauuccuacuucuuacgccccccacaucacccgccucgagaccucaaggguagaggugggcacccccgccuccgcacuuuugcucggggcuccagauuguagggcagggcggcgcuucucggaaagcgaaagccggcggggcggggcgggugccgcaggagaaagaggaagcgcuggcagacaaugcgacccgaccgcgcugaggcuccaggaccgcccgccauggcugcaggaggucccggcgcggggucugcggccccggucuccuccacauccucccuuccccuggcugcucucaacaugcgagugcggcgccgccugucucuguucuugaacgugcggacacagguggcggccgacuggaccgcgcuggcggaggagauggacuuugaguacuuggagauccggcaacuggagacacaagcggaccccacuggcaggcugcuggacgccuggcagggacgcccuggcgccucuguaggccgacugcucgagcugcuuaccaagcugggccgcgacgacgugcugcuggagcugggacccagcauugggcauaugccugagcguuucgaugccuucaucugcuauugccccagcgacauccaguuugugcaggagaugauccggcaacuggaacagacaaacuaucgacugaaguugugugugucugaccgcgauguccugccuggcaccugugucuggucuauugcuagugagcucaucgaaaagaggugccgccggaugguggugguugucucugaugauuaccugcagagcaaggaaugugacuuccagaccaaauuugcacucagccucucuccaggugcccaucagaagcgacugauccccaucaaguacaaggcaaugaagaaagaguuccccagcauccugagguucaucacugucugcgacuacaccaaccccugcaccaaaucuugguucuggacucgccuugccaaggccuugucccugcccugaagacuguucugaggcccuggguguguguguaucugucugccuguccauguacuucugcccugccuccuccuuucguuguaggaggaaucugugcucuacuuaccucucaauuccuggagaugccaacuucacagacacgucugcagcagcuggacaucacauuucauguccugcauggaaccaguggcugugaguggcauguccacuugcuggauuaucagccaggacacuauagaacaggaccagcugagacuaagaaggaccagcagagccagcucagcucugagccauucacacaucuucacccucaguuuccucacuugaggagugggauggggagaacagagaguagcuguguuugaaucccuguaggaaauggugaagcauagcucugggucuccugggggagaccaggcuuggcugcgggagagcuggcuguugcuggacuacaugcuggccacugcugugaccacgacacugcuggggcagcuucuuccacagugaugccuacugaugcuucagugccucugcacaccgcccauuccacuuccuccuuccccacagggcagguggggaagcaguuuggcccagcccaaggagaccccaccuugagccuuauuuccuaauggguccaccucucaucugcaucuuucacaccucccagcuucugcccaaccuucagcagugacaaguccccaagagacucgccugagcagcuugggcugcuuuucauuuccaccugucaggaugccuguggucaugcucucagcuccaccuggcaugagaagggauccuggccucuggcauauucaucaaguaugaguucuggggaugagucacuguaaugaugugagcagggagccuuccucccugggccaccugcagagagcuuucccaccaacuuuguaccuugauugccuuacaaaguuauuuguuuacaaacagcgaccauauaaaagccuccugccccaaagcuugugggcacaugggcacauacagacucacauacagacacacacauauauguacagacauguacucucacacacacaggcaccagcauacacacguuuuucuagguacagcucccaggaacagcuaggugggaaagucccaucacugagggagccuaaccaugucccugaacaaaaauugggcacucaucuauuccuuuucucuugugucccuacucauugaaaccaaacucuggaaaggacccaauguaccaguauuuauaccucuaaugaagcacagagagaggaagagagcugcuuaaacucacacaacaaugaacugcagacacagcuguucucucccucucuccuucccagagcaauuuauacuuuacccucaggcuguccucuggggagaaggugccauggucuuaggugucugugccccaggacagacccuaggacccuaaauccaauagaaaaugcauaucuuugcuccacuuucagccaggcuggagcaagguaccuuuucuuaggaucuugggagggaauggaugccccucucugcaugaucuuguugaggcauuuagcugccaugcaccugucccccuuuaauacugggcauuuuaaagccaucucaagaggcaucuucuacauguuuuguacgcauuaaaauaauuucaaagauaucugagaaaagccgauauuugccauucuuccuauauccuggaauauaucuugcauccugaguuuauaauaauaaauaauauucuaccuuggaaaaaaaaaaaaaaa

SEQ ID NO: 9 - RNA, 19 nt, Artificial Sequence
  Chemically synthesized
cgugcaaagugguauccug

SEQ ID NO: 10 - RNA, 19 nt, artificial
  Chemically synthesized
caggauaccacuuugcacg

SEQ ID NO: 11 - RNA, 19 nt, artificial
  Chemically synthesized
ggguucuaugauauuugua

SEQ ID NO: 12 - RNA, 19 nt, artificial
  Chemically synthesized
uacaaauaucauagaaccc

SEQ ID NO: 13 - RNA, 19 nt, artificial
  Chemically synthesized
ggguaaaggcuacuuugau

SEQ ID NO: 14 - RNA, 19 nt, artificial
  Chemically synthesized
aucaaaguagccuuuaccc

SEQ ID NO: 15 - RNA, 19 nt, artificial
  Chemically synthesized
gaaugugacuuccagacca

SEQ ID NO: 16 - RNA, 19 nt, artificial
  Chemically synthesized
uggucuggaagucacauuc

SEQ ID NO: 17 - DNA, 51 bp, artificial
  Chemically synthesized
gtcgtatccagtgcagggtccgaggtattcgcactggatacgaccgtgcaa

SEQ ID NO: 18 - DNA, 21 bp, artificial
  Chemically synthesized
cggcggcaggataccactttg

SEQ ID NO: 19 - DNA, 20 bp, artificial
  Chemically synthesized
agtgcagggtccgaggtatt

SEQ ID NO: 20 - DNA, 25 bp, artificial
  Chemically synthesized
cgactggagcacgaggacactgcat

SEQ ID NO: 21 - DNA, 25 bp, Artificial Sequence
  Chemically synthesized
ggacactgcatggactgaaggagta

(19) United States
(12) Patent Application Publication
(10) Pub. No.: US 2016/0208247 A1
(43) Pub. Date: Jul. 21, 2016

(54) METHODS OF USE OF SPHINGOLIPID POLYALKYLAMINE OLIGONUCLEOTIDE COMPOUNDS

(71) Applicant: QBI ENTERPRISES LTD., Nes Ziona (IL)

(72) Inventors: Elena FEINSTEIN, Rehovot (IL); Sharon AVKIN-NACHUM, Nes Zionna (IL)

(73) Assignee: QBI ENTERPRISES LTD., Nes Ziona (IL)

(21) Appl. No.: 14/908,132

(22) PCT Filed: Jul. 30, 2014
(86) PCT No.: PCT/IL2014/050693

§ 371 Date: Jan. 28, 2016

Related U.S. Application Data
(60) Provisional application No. 61/860,275, filed on Jul. 31, 2013.

(51) Int. Cl. C12N 15/113

(52) U.S. Cl. CPC C12N 15/113; C12N 2310/14; C12N 2310/351; C12N 2320/30

(57) ABSTRACT

Provided herein are sphingolipid-polyalkylamine siRNA compounds pharmaceutical compositions comprising such compounds, and methods of use in therapy.

### RELATED APPLICATIONS

This application claims the benefit of U.S. Provisional Application Ser. No. 61/860,275 filed Jul. 31, 2013, entitled “Methods of Use of Sphingolipid Polyalkylamine Oligonucleotide Compounds” and incorporated herein by reference in its entirety and for all purposes.

### SEQUENCE LISTING

This application incorporates-by-reference nucleotide and/or amino acid sequences which present in the file named “253-PCT1.ST25.txt”, which is 29 kb in size, and which was created on Jul. 27, 2014 in the IBM-PC machine format, having an operating system compatibility with MS-Windows, and is submitted herewith.

### FIELD OF THE INVENTION

Disclosed herein are methods of using sphingolipid-polyalkylamine oligonucleotide compounds for the modulation of gene expression in therapy. The compounds, which include single stranded or double-stranded nucleic acid molecules, show improved cell penetration and enhanced circulation time compared to non-conjugated compounds and are useful in therapeutic treatment of subjects suffering from diseases or conditions in which modulation of gene expression provides a favorable outcome.

### BACKGROUND OF THE INVENTION

Use of therapeutic oligonucleotides, including single stranded (ssNA) and double-stranded nucleic acids (dsNA including dsRNA and siRNA), in the clinic has been hampered by the lack of efficient and safe delivery systems. Cationic lipids have been used to deliver therapeutic oligonucleotides, however, their use is limited by cell toxicity and the fact that cationic lipids accumulate primarily in the liver.

International Patent Publication Nos. WO 2008/104978, WO 2009/044392, WO 2011/066475, WO 2011/084193, WO 2011/085056, WO/2012/078536, assigned to the assignee of the present invention, disclose chemically modified dsRNA, and are hereby incorporated by reference in their entirety.

Sphingolipid-polyalkylamine conjugates are disclosed in U.S. Pat. No. 7,771,711; and a process for large-scale preparation of sphingosine is provided in U.S. Pat. No. 6,469,148; both are incorporated by reference in their entirety.

PCT publication No. WO 2010/150004 relates to oligonucleotides carrying lipid molecules and their use as inhibitors of gene expression.

The applicants of this patent application are co-applicants of a patent application concurrently filed with the instant patent application, claiming the benefit of U.S. Provisional Application Ser. No. 61/860,274 filed Jul. 31, 2013, and disclosing synthesis of sphingolipid-polyalkylamine derivatives and their use in generating sphingolipid-polyalkylamine oligonucleotides.

There remains a need for active and safe dsNA therapeutic agents, which exhibit at least one of improved cellular uptake, enhanced endosomal release, favorable biodistribution, increased circulation time, reduced toxicity and reduced immunogenicity compared to the unmodified counterparts, while retaining therapeutic activity.

### SUMMARY OF THE INVENTION

Provided herein are methods of using sphingolipid-polyalkylamine oligonucleotide compounds, and in particular sphingolipid-polyalkylamine single and double stranded nucleic acid compounds useful in therapy, with the proviso that the therapy does not comprise treatment of cancer. The methods of treatment and compounds and compositions for use disclosed herein are preferably by local administration of a sphingolipid-polyalkylamine oligonucleotide to a target tissue or organ.

Further provided are sphingolipid-polyalkylamine oligonucleotide compounds or salt of such compounds or composition comprising such compounds or salt of such compounds, for use in therapy, with the proviso that the therapy does not comprise treatment of cancer.

Further provided is use of the sphingolipid-polyalkylamine oligonucleotide compounds or salt of such compounds for the manufacture of a medicament for the treatment of a disease or disorder, with the proviso that the disease or disorder does not comprise cancer.

The sphingolipid-polyalkylamine oligonucleotide compounds disclosed herein possess structures and modifications which, for example, exhibit at least one of increased cellular uptake, increased circulation time, enhanced endosomal release, improved biodistribution, reduced toxicity, reduced immunogenicity, reduced off-target effects, or enhanced loading into the RISC complex when compared to an unmodified and/or unconjugated nucleic acid molecule.

In one aspect, provided herein is a method for treating or preventing a disease or a disorder in a subject having or at risk of developing the disease or disorder comprising administering to the subject a therapeutic amount of a sphingolipid-polyalkylamine-oligonucleotide compound comprising an oligonucleotide and a sphingolipid-polyalkylamine conjugate, having general formula I:

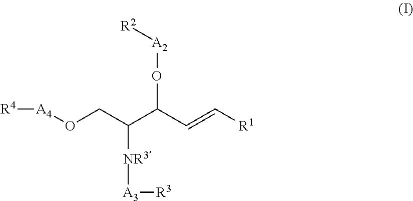

wherein
R¹ is a branched or linear C₇-C₂₄ alkyl, alkenyl or polyenyl;
R², R³ and R⁴ each independently is hydrogen, C₁-C₄ alkyl, a branched or linear polyalkylamine or derivative thereof, or an oligonucleotide;
R^{3′} is hydrogen or C₁-C₄ alkyl;
A₂, A₃ and A₄ each independently is present or absent but if present is one of C(O), C(O)NHX, C(O)NHR⁵X, C(O)R⁵X, C(O)R⁵C(O)X, R⁵X or R⁵OC(O)X;
R⁵ is a branched or linear C₁-C₂₀ alkyl chain optionally substituted with one or more heteroatoms;
X is present or absent but if present is S, P, O or NH;
at least one of R², R³ or R⁴ is a branched or linear polyalkylamine or derivative thereof; and
at least one of R², R³ or R⁴ is an oligonucleotide;
or a salt of such compound, with the proviso that the disease or disorder is other than cancer.

In one aspect, provided is a compound of Formula I for use in treating or preventing a disease or a disorder, with the proviso that the disease or a disorder is other than cancer.

In another aspect, provided is use of a compound of Formula I for the preparation of a medicament for treating or preventing a disease or a disorder, with the proviso that the disease or a disorder is other than cancer.

In some embodiments of the method, compound for use or use, the oligonucleotide is capable of modulating expression of a target gene (e.g. ncRNA, mRNA etc. . . . ). In some embodiments, the oligonucleotide down regulates expression of the target gene. In other embodiments, the oligonucleotide up regulates expression of the target gene.

In some embodiments of the method, compound for use or use, the disease or disorder is selected from the group consisting an inner ear disease or disorder, an eye disease or disorder, a respiratory disease or disorder, a central or peripheral nervous system disease or disorder, a skin disease or disorder, a renal disease or disorder, a cardiac disease or disorder, a liver disease or disorder and an inflammatory, a viral infection, a bacterial infection a fungal infection or fibrotic disease or disorder of any organ.

In some embodiments of the method, compound for use or use, the inner ear disease or disorder is a hearing loss or a vestibular disease or disorder. In preferred embodiments, the sphingolipid-polyalkylamine oligonucleotide compound or salt of such compound is formulated for otic or transtympanic delivery. In some embodiments, the inner ear disease or condition is a hearing loss or disorder or a vestibular or disorder (e.g. balance disorder, tinnitus and the like), and wherein the sphingolipid-polyalkylamine oligonucleotide compound or salt of such compound is to be administered to the subject via a local otic administration route selected from ear drops to the tympanic membrane, transtympanic delivery to the middle ear for subsequent diffusion through the round window membrane, and intraoperational delivery methods to the round window or to the endolymph.

In some embodiments of the method, compound for use or use, the disease or disorder is an eye disease or disorder. In preferred embodiments, the sphingolipid-polyalkylamine oligonucleotide compound or salt of such compound is formulated for ocular or intravitreal delivery. In some embodiments, the disease or condition is an eye disease or disorder, and the sphingolipid-polyalkylamine oligonucleotide compound or salt of such compound is to be administered to the subject via local ocular administration routes or local otic administration route selected from eye drops, intravitreal, subretinal or subscleral administration.

In some embodiments of the method, compound for use or use, the disease or disorder is a respiratory disease or disorder. In some embodiments, the sphingolipid-polyalkylamine oligonucleotide compound or salt of such compound is formulated for pulmonary, e.g. instillation, inhalation or intratracheal delivery. In some embodiments, the disease or condition is a respiratory disease or disorder and the sphingolipid-polyalkylamine oligonucleotide compound or salt of such compound is to be administered to the subject via one of the local pulmonary administration routes selected from intranasal, intratracheal and/or intrabronchial instillation or inhalation.

In some embodiments, the disease or condition is selected from the group consisting of a central or peripheral nervous system disease or disorder; and the sphingolipid-polyalkylamine oligonucleotide compound or salt of such compound is to be administered to the subject via one of the local administration routes selected from intraotic, intrathecal/suprathecal or intraventricular administration.

In some embodiments of the method, compound for use or use, the disease or disorder a skin disease or disorder. In some embodiments, the sphingolipid-polyalkylamine oligonucleotide compound or salt of such compound is formulated for topical or intradermal delivery. In some embodiments, the disease or condition is a skin disease or disorder; and wherein the sphingolipid-polyalkylamine oligonucleotide compound or salt of such compound is to be administered to the subject via one of the local administration routes including but not limited to topical, subcutaneous or intradermal administration.

In some embodiments, the disease or condition is selected from the group consisting of a cardiac disease or disorder; and the sphingolipid-polyalkylamine oligonucleotide compound or salt of such compound is to be administered to the subject via one of the local administration routes including but not limited to intramyocardial or intracoronary administration.

In some embodiments, the disease or condition is a renal disease or disorder, a cardiac disease or disorder, a liver disease or disorder and an inflammatory or fibrotic disease or disorder of any location; and the sphingolipid-polyalkylamine oligonucleotide compound or salt of such compound is to be administered to the subject via parenteral administration selected from the group consisting of intravenous, intraarterial, subcutaneous, transdermal, intraperitoneal, or intramuscular administration.

In some embodiments of the method, compound for use or use, in the sphingolipid-polyalkylamine-oligonucleotide compound R¹ is C₇-C₂₄ alkyl, C₁₀-C₂₀ alkyl or C₁₀-C₁₆ alkyl. Preferably R¹ is C₁₃ alkyl.

In some embodiments of the method, compound for use or use, in the sphingolipid-polyalkylamine-oligonucleotide compound, the sphingolipid is sphingosine.

In some embodiments of the method, compound for use or use, in the sphingolipid-polyalkylamine-oligonucleotide compound A₂ is C(O). In some embodiments, A₄ is C(O). In some embodiments R² is a linear polyalkylamine or a derivative thereof. In some embodiments R² is a linear polyalkylamine or a derivative thereof. Preferably the linear polyalkylamine is spermine or spermidine. In some embodiments R² is spermidine. In some embodiments R² is spermine

In some embodiments of the method, compound for use or use, in the sphingolipid-polyalkylamine oligonucleotide compound R^{3′} is hydrogen, A₂ is C(O), A₃ is absent and R² is spermine and provided herein is a compound having general formula (Ia)

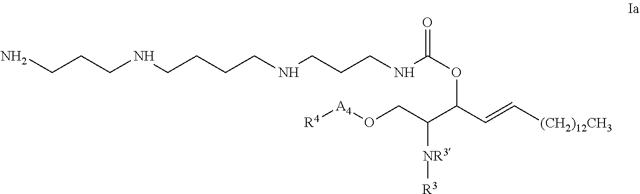

wherein
A₄ is one of C(O), C(O)NHX, C(O)NHR⁵X, C(O)R⁵X, C(O)R⁵C(O)X, R⁵X or R⁵OC(O)X;
R⁵ is a branched or linear C₁-C₂₀ alkyl chain optionally substituted with one or more heteroatoms;
R³ and R^{3′} each independently is hydrogen or C₁-C₄ alkyl;
R⁴ is an oligonucleotide;
or a salt of such compound.

In some embodiments R^{3′} is hydrogen, A₂ is C(O), A₃ is absent and R² is spermidine and provided herein is a compound having general formula (Ib)

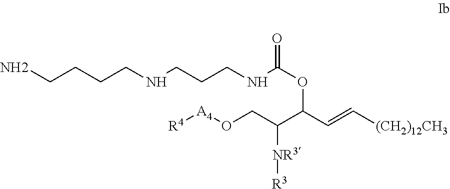

wherein
A₄ is one of C(O), C(O)NHX, C(O)NHR⁵X, C(O)R⁵X, C(O)R⁵C(O)X, R⁵X or R⁵OC(O)X;
R⁵ is a branched or linear C₁-C₂₀ alkyl chain optionally substituted with one or more heteroatoms;
R³ and R^{3′} each independently is hydrogen or C₁-C₄ alkyl;
R⁴ is an oligonucleotide;
or a salt of such compound.

In some embodiments, in the compound A₄ is C(O), R¹ is C₁₃ alkyl, R⁴ is spermine or spermidine and provided herein is a compound having general formula (Ic) or (Id):

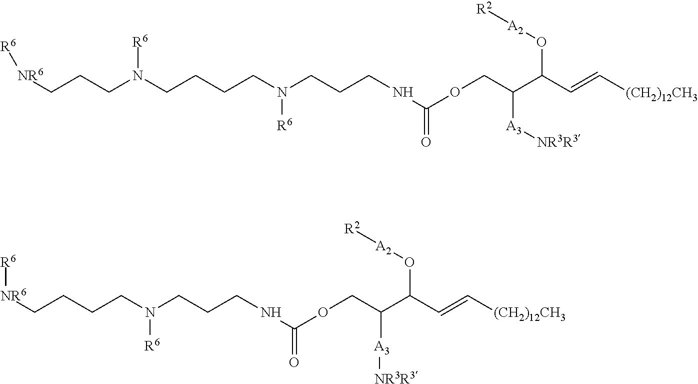

In various embodiments of general formulae Ia and Ib, A₄ is C(O)NHR⁵X, wherein R⁵ is a linear C₆ alkyl chain and X is O, having the general formula IIa or IIb:

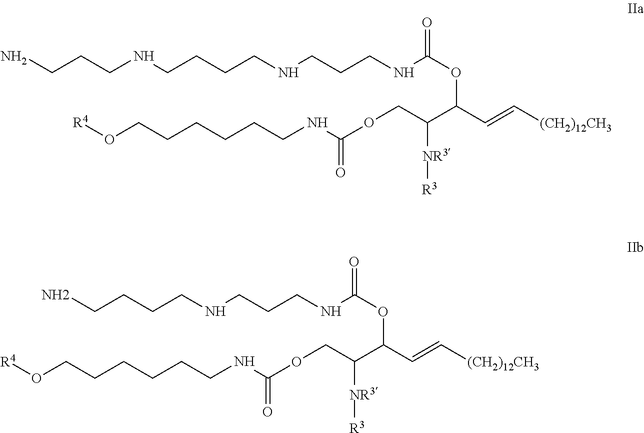

wherein R⁴ is an oligonucleotide.

In some embodiments of the method, compound for use or use, the sphingolipid-polyalkylamine oligonucleotide compounds are useful in therapy, with the proviso that the therapy is not cancer therapy. In some embodiments the oligonucleotide is a single stranded oligonucleotide. Preferred single-stranded oligonucleotides include an antisense nucleic acid molecule of any functional type or composition targeting mRNA, pre-mRNA or any type of non-coding RNA, an aRNA, an aptamer, a ribozyme, a synthetic mRNA and shRNA. In some embodiments of the method, compound for use or use, the oligonucleotide is a dsNA molecule.

In certain embodiments, the dsNA acts to up regulate target gene expression and is, for example, a short activating RNA (saRNA). A preferred dsNA is a siNA molecule, preferably a chemically modified siNA molecule. In some embodiments, the dsNA acts via RNA interference. The double stranded NA (dsNA) molecule of any type or composition selected from the group consisting of but not limited to a siRNA, a miRNA and a miRNA mimetic.

In some embodiments, one or more of the ribonucleotides in the siRNA is substituted with a modified ribonucleotide, an unconventional moiety or both a modified ribonucleotide and an unconventional moiety.

In some embodiments of the method, compound for use or use, the siRNA molecule comprises a sense strand and an antisense strand each strand having a 5′ terminus and a 3′terminus;

(a) wherein the sense strand is 8 to 49 nucleotides in length and the antisense strand is 15 to 49 nucleotides in length;

(b) a 15 to 49 nucleotide sequence of the antisense strand is complementary to a consecutive sequence of a target gene RNA;

(c) an 8 to 49 nucleotide sequence of the sense strand is complementary to the antisense strand and includes an 8 to 49 nucleotide sequence of an mRNA of a target gene;

(d) wherein a sphingolipid-polyalkylamine conjugate is covalently attached to the 5′ terminus or the 3′ terminus of the sense strand or the 5′ terminus or the 3′ terminus of the antisense strand.

In some embodiments, one or more of the ribonucleotides in the siRNA is substituted with a modified ribonucleotide, an unconventional moiety or both a modified ribonucleotide and an unconventional moiety. In preferred embodiments, the sense strand, the antisense strand or both strands include at least one unconventional moiety or a non-nucleotide overhang.

Each nucleotide is independently unmodified (natural ribonucleotides) or modified ribonucleotides (2′O-alkyl, 2′deoxyfluoro) or an unconventional moiety (L-DNA, L-RNA, TNA, 2′5′ linked, UNA and the like).

In some embodiments the sense strand comprises two or more sets of covalently joined consecutive nucleotides which are not joined by a covalent bond (ie the sense strand is “nicked”).

In some embodiments the dsNA is a siRNA molecule having the structure set forth as A1 below

### A1

5′ (N)x-Z 3′ (antisense strand)

3′ Z′—(N′)y-z″ 5′ (sense strand)

wherein each of N and N′ is an unmodified ribonucleotide, a modified ribonucleotide or an unconventional moiety;
wherein each of (N)x and (N′)y is an oligonucleotide in which each consecutive N or N′ is joined to the next N or N′ by a covalent bond;
wherein each of x and y is independently an integer between 15 and 49;
wherein z″ is present or absent, but if present is a capping moiety covalently attached to the 5′ terminus of the sense strand;
wherein each of Z and Z′ is independently present or absent, but if present is 1-5 consecutive nucleotides or non-nucleotide moieties or a combination thereof are covalently attached at the 3′ terminus of the strand in which it is present;
wherein the sense strand, the antisense strand or both strands include at least one unconventional moiety or a non-nucleotide overhang;
wherein a sphingolipid-polyalkylamine conjugate is covalently attached to at least one of the 3′ terminus of the antisense strand, the 3′ terminus of the sense strand, the 5′ terminus of the sense strand or the 5′ terminus of the antisense strand; and
wherein the sequence of (N′)y is substantially complementary to the sequence of (N)x; and wherein (N)x comprises an antisense sequence complementary to a consecutive sequence in a target RNA.

In some embodiments, z″ is absent when the sphingolipid-polyalkylamine conjugate is attached at the 5′ terminus of the sense strand, and/or, Z′ is absent when the sphingolipid-polyalkylamine is attached at the 3′ terminus of the sense strand then; and/or Z is absent when the sphingolipid-polyalkylamine is attached at the 3′ terminus of the antisense strand.

In some embodiments, more than one of N and N′ is a modified ribonucleotide or an unconventional moiety.

In some embodiments of the siRNA, each covalent bond joining each consecutive N or N′ is independently selected from a phosphodiester bond or a phosphodiester bond.

In certain embodiments of the siRNA, x=y and each of x and y is an integer from 15-49, or from 17-40, preferably from 18-25. In some embodiments, x=y=19, 20, 21, 22 or 23. Preferably x=y=19 or 21. In certain embodiments, x=y=19. In certain embodiments, x is an integer from 19-25 and y is an integer from 15-17.

In some embodiments of the method, compound for use, and use, in the sphingolipid-polyalkylamine oligonucleotide compound, the sphingolipid-polyalkylamine conjugate is covalently attached to at least one of the 3′ terminus of the sense strand (N′)y, the 3′ terminus of the antisense strand (N)x or the 5′ terminus of the sense strand (N′)y. In some embodiments, the sphingolipid-polyalkylamine conjugate is covalently attached to the 3′ terminus of (N)x. In some embodiments, the sphingolipid-polyalkylamine conjugate is covalently attached to the 3′ terminus of (N′)y. The 3′ terminus of (N)x or (N′)y may include Z or Z′, respectively, for example a nucleotide or non-nucleotide overhang. In some embodiments, the sphingolipid-polyalkylamine conjugate is attached to the strand at the Z or Z′. Such compounds may further include a capping moiety (z″) covalently attached to the 5′ terminus of the sense strand (N′)y. In some embodiments, the sphingolipid-polyalkylamine conjugate may be attached to the 5′ terminus of the sense strand, for example when the oligonucleotide is a DICER substrate dsNA.

In preferred embodiments, the sphingolipid-polyalkylamine conjugate is covalently attached to the 5′ terminus of (N′)y. In such compounds, Z and/or Z′ is optionally covalently attached at the 3′ terminus of (N)x and/or at the 3′ terminus of (N)y.

In some embodiments, the sequence of (N′)y is fully complementary to the sequence of (N)x, and the sequence of (N)x is fully complementary to the target RNA. The sequence of (N′)y may also be fully complementary to the sequence of (N)x and the sequence of (N)x is partially complementary to the target RNA. In such compounds, for example, the 5′ terminal nucleotide of the antisense strand [(N)x] is mismatched to the target RNA. Such a structure is set forth as A2 below:

A2 5′ N1-(N)x-Z 3′ (antisense strand)

3′ Z′—N2-(N′)y-z″ 5′ (sense strand)

wherein each of N2, N and N′ is an unmodified nucleotide, a modified nucleotide, nucleotide analogue or an unconventional moiety;
wherein each of (N)x and (N′)y is an oligonucleotide in which each consecutive N or N′ is joined to the adjacent N or N′ by a covalent bond;
wherein x is an integer from 8 to 48 and y is an integer from 15 to 48;
wherein the sequence of N2-(N′)y has complementarity to the sequence of N1-(N)x and (N)x has complementarity to a consecutive sequence in a target RNA;
wherein N2 is covalently bound to (N′)y;
wherein N1 is covalently bound to (N)x and is mismatched to the target mRNA;
wherein N1 is a moiety selected from the group consisting of natural uridine, a modified uridine, deoxyribouridine, ribothymidine, deoxyribothymidine, natural adenosine, modified adenosine, deoxyadenosine, adenosine pyrazolotriazine nucleic acid analogue, deoxyadenosine and pyrazolotriazine nucleic acid analogue,
wherein z″ is a capping or conjugate moiety and may be present or absent, but if present is covalently attached at the 5′ terminus of N2-(N′)y; and
wherein Z is present or absent, but if present is 1-5 consecutive nucleotides, 1-5 consecutive nucleotide analogues or 1-5 consecutive non-nucleotide moieties or a conjugate moiety covalently attached at the 3′ terminus of the antisense strand;
wherein Z′ is present or absent, but if present is 1-5 consecutive nucleotides, 1-5 consecutive nucleotide analogues or 1-5 consecutive non-nucleotide moieties or a complex moiety covalently attached at the 3′ terminus of the sense strand; and
wherein at least a portion of the sequence of (N)x is complementary to a consecutive sequence in the target RNA; or a pharmaceutically acceptable salt of such molecule.

Molecules fitting the description of Structure (A2) are also referred to herein as “18+1” or “18+1 mer”.

The siRNA is unmodified or chemically modified, preferably chemically modified. For example, in the chemically modified siRNA, at least one of N or N′ is a modified ribonucleotide, wherein the modified ribonucleotide possesses a modification in the sugar moiety, in the base moiety or in the internucleotide linkage moiety. In some embodiments, at least one modified ribonucleotide comprises a 2′ sugar modification. In some embodiments, the 2′ sugar modification is selected from the group consisting of 2′O-alkyl sugar modification, for example a 2′O-methyl sugar modification, 2′deoxyfluoro sugar modification, 2′-O-methoxyethyl (2′MOE) sugar modification and a 2′-amino sugar modification. In some embodiments, one or more up to about 20 N and N′ is a 2′O-methyl sugar modified ribonucleotide. In some embodiments, each internucleotide linkages (i.e. covalent bonds joining N or N′ is joined to the adjacent N or N′) is a phosphodiester linkage. In some embodiments, one or more internucleotide linkage comprises a phosphorothioate linkage.

In some embodiments, one or more up to about 12 of N and N′ in the chemically modified siRNA is an unconventional moiety. An unconventional moiety may be for example, a mirror nucleotide (i.e. L-DNA or L-RNA), a nucleotide forming a 2′-5′ linkage (2′5′ nucleotide), a locked nucleic acid (LNA), an unlocked nucleic acid (UNA), a threose nucleic acid (TNA), a DNA and the like.

In some embodiments, at least one unconventional nucleotide is present in (N′)y, the unconventional moiety selected from a 2'S′ linked nucleotide (i.e. 2′5′ linked RNA or 2′5′ linked DNA), a threose nucleic acid (TNA), pyrazolotriazine nucleotide or a mirror nucleotide (i.e. L-DNA or L-RNA). In some embodiments, x=y=19 and a 2′5′ linked nucleotide is present in positions (5′>3′) 16, 17, 18, and 19 or in positions 15, 16, 17, 18, and 19. In some embodiments, the compound further comprises a 2′5′ linked nucleotide, a threose nucleic acid (TNA) or a mirror nucleotide (i.e. L-DNA or L-RNA) in at least one of positions 6, 7, or 8 in (N)x. preferably the compound comprises a 2′5′ linked nucleotide or a TNA in position 7. IN some embodiments, least one of N or N′ is a sugar modified ribonucleotide. In preferred embodiments, the sugar modification comprises a 2′ sugar modification, selected from the group consisting of 2′O-methyl sugar modification, 2′deoxyfluoro sugar modification, 2′-O-methoxyethyl (2′MOE) sugar modification and a 2′-amino sugar modification, preferably a 2′O-methyl sugar modification.

In some embodiments, the sequence of (N′)y is fully complementary to the sequence of (N)x, and the sequence of (N)x is fully complementary to the target RNA. In some embodiments, the sequence of (N′)y is fully complementary to the sequence of (N)x and the sequence of (N)x is partially complementary to the target RNA. In some embodiments, the sequence of (N′)y is partially complementary to the sequence of (N)x and the sequence of (N)x is partially complementary to the target RNA. For example, the dsRNA compound includes mismatches or insertions of 2-6 nucleotides between the two strands of the duplex.

In preferred embodiments, the 5′ terminal nucleotide of the antisense strand [(N)x] is mismatched to the target RNA.

The target RNA may be endogenous or exogenous RNA, representing either coding or non-coding RNA. In some embodiments, the target RNA is the transcription product of an endogenous mammalian gene, that is, for example, up regulated in a pathological state. In certain preferred embodiments, the target RNA is mammalian mRNA, preferably human mRNA. In some embodiments, the target RNA is prokaryotic RNA, for example, a viral, fungal or bacterial RNA. In some embodiments, the target RNA is IncRNA.

In some embodiments the sphingolipid-polyalkylamine oligonucleotide compound is formulated with a carrier. In preferred embodiments the carrier is a pharmaceutically acceptable carrier. In some embodiments, the composition is formulated for parenteral or enteral administration. In preferred embodiments, the parenteral administration is selected from the group consisting of intravenous, subcutaneous, transdermal and intramuscular administration. In some embodiments the composition is formulated for topical administration, for example, for intradermal application. In some embodiments the composition is formulated for intravitreal or otic postoperative (e.g. transtympanic or topical) administration.

In another aspect, provided herein is a method of generating a plant with an altered phenotype, comprising contacting a plant cell with a sphingolipid-polyalkylamine oligonucleotide compound disclosed herein and generating a plant from the plant cell. In preferred embodiments the target RNA is plant RNA and the compounds are useful in generating plants with altered traits or treating a plant disease.

This disclosure is intended to cover any and all adaptations or variations of combination of features that are disclosed in the various embodiments herein. Although specific embodiments have been illustrated and described herein, it should be appreciated that the invention encompasses any arrangement of the features of these embodiments to achieve the same purpose. Combinations of the above features, to form embodiments not specifically described herein, will be apparent to those of skill in the art upon reviewing the instant description.

### BRIEF DESCRIPTION OF THE FIGURES

FIG. 1 is a graph showing dose-dependent knockdown of Renilla Luciferase activity by sphingolipid polyalkylamine siRNA compound target HES5 but not for their non-conjugated counterparts.

FIG. 2 is a picture of PAGE showing gel migration patterns of sphingolipid-spermine/sphingolipid spermidine conjugated Rac1 siRNA compounds and non conjugated siRAC1 compounds on a non-denaturing polyacrylamide gel.

FIGS. 3A and 3B show accumulation of sphingolipid-spermine/sphingolipid spermidine conjugated siRAC1 compounds and non conjugated siRAC1 compounds in rat retina following intravitreal injection of different amounts of compounds (2 ug, 6 ug and 20 ug per eye).

FIG. 4 shows distribution of sphingolipid-spermine/sphingolipid spermidine conjugated siRAC1 compounds and non conjugated siRAC1 compounds in retinal section as analyzed by siRNA in situ hybridization (siISH) images of retinal sections following intravitreal administration of the compounds to the eye. A and B) unconjugated siRAC1 and vehicle do not enter the inner layers of the retina; C and D) sphingolipid-spermine/sphingolipid spermidine siRAC1 compounds are detected in the inner layers of the retina.

FIG. 5A shows a bar graph of RAC1 knock down in the retina by sphingolipid-spermine conjugated siRAC1 compound compared to non conjugated siRAC1. FIG. 5B shows the RACE product of sphingolipid-spermine siRAC1 compound. FIG. 5C is a graph showing changes in expression of IFN-responsive genes IFIT and MX1 following IVT injection of SL-spermine linked siRAC1 compound. In each pair of columns, Left column represents levels of IFIT, right column represents levels of MX1.

FIG. 6 is a bar graph showing RAC1 mRNA levels per retina after 1, 3 or 7 days post IVT injection of SL-spermine linked siRAC1 compound (2 ug/eye, 6 ug/eye or 20 ug/eye).

FIG. 7 is a bar graph showing levels of residual RAC1 mRNA in the retina following local treatment with sphingolipid-spermine and sphingolipid spermidine siRAC1 compound (2 ug or 20 ug) in the eye.

FIG. 8 is a bar graph showing levels of residual RAC1 mRNA in the inner ear following local treatment with sphingolipid-spermine siRAC1 compound after 1 and 3 days.

FIG. 9 is a bar graph showing levels of sphingolipid-spermine siRAC1 compound in the lung 24 hours following intratracheal administration.

FIGS. 10A-10C shows knockdown of RAC1 mRNA in mice lungs following intratracheal administration of sphingolipid spermine and sphingolipid spermidine siRAC1 compound. FIG. 10A shows RAC1 mRNA quantity per mg lung tissue (presented as % of residual levels in vehicle treated lungs) of sphingolipid spermine and sphingolipid spermidine siRAC1 compound compared to treatment with non conjugated siRNA. FIG. 10B shows that the specific RT-PCR (RACE) product predicted for RNAi-mediated cleavage of RAC1 mRNA by sphingolipid spermine or sphingolipid spermine siRAC1 compounds was generated in mouse lung tissue. FIG. 10C is a bar graph that shows that sphingolipid spermine or sphingolipid spermidine siRAC1 compound did not induce the IFN-responsive genes.

The compounds, methods, materials, and examples that will now be described are illustrative only and are not intended to be limiting; materials and methods similar or equivalent to those described herein can be used in practice or testing of the invention. Other features and advantages of the invention will be apparent from the following detailed description, and from the claims.

### DETAILED DESCRIPTION OF THE INVENTION

Disclosed herein, inter alia, are sphingolipid-polyalkylamine oligonucleotide compounds comprising a sphingolipid-polyalkylamine conjugate covalently linked to a single stranded or double stranded oligonucleotide molecule, preferably a chemically modified siRNA molecule useful for modulating expression of a target gene, particularly to modulating expression of a target gene for treatment of a disease or disorder other than cancer. The sphingolipid-polyalkylamine oligonucleotide compounds disclosed herein exhibit one or more of increased on-target activity, decreased off-target activity, enhanced uptake into cells accompanied with enhanced endosomal escape into the cytoplasm, increased nuclease stability (exonuclease and or endonuclease), and reduced immunomodulation when compared to an unmodified double-stranded nucleic acid compound. Without wishing to be bound to theory, the presence of a sphingolipid-polyalkylamine conjugate provides stability to the oligonucleotide in body fluids and facilitates endosomal escape, by creation of a ‘proton sponge effect’ in the endosome.

Furthermore, it is now disclosed for the first time that sphingolipid-polyalkylamine oligonucleotide compounds, exemplified by short double stranded NA molecules, conjugated to a sphingolipid-spermidine or sphingolipid-spermine conjugate moiety have improved tissue retention in bodily tissues, including optic and otic tissues, thus leading to high concentrations of the active ingredient upon local administration. Additionally, it was shown that sphingolipid-polyalkylamine moiety conjugated dsNA exhibited broad distributed within the retinal layers, showing higher accumulation as compared to their non-conjugated counterparts as well as accessibility into retinal layers in which the non-conjugated counterparts could not be detected, e.g. into rods and cones, RPE and choroid.

The sphingolipid-polyalkylamine oligonucleotide compounds and compositions are able to modulate gene expression, for example down regulate, knock down, attenuate, reduce or inhibit target gene expression and are useful in the treatment of subjects suffering from diseases or conditions and or symptoms associated with such diseases or conditions or at risk of contracting diseases or conditions in which gene expression has adverse consequences

### DEFINITIONS

It is to be noted that, as used herein, the singular forms “a”, “an” and “the” include plural forms unless the content clearly dictates otherwise. Where aspects or embodiments of the invention are described in terms of Markush groups or other grouping of alternatives, those skilled in the art will recognize that the invention is also thereby described in terms of any individual member or subgroup of members of the group.

A “compound” and a “molecule” are used interchangeably herein when referring to the sphingolipid-polyalkylamine oligonucleotide.

The term “inhibit” as used herein refers to reducing the expression of a gene including reducing coding or non-coding transcript of a gene, or the activity of the product of such gene to an extent sufficient to achieve a desired biological or physiological effect Inhibition is either complete or partial.

A “siNA inhibitor” “dsRNA inhibitor” or “dsRNA molecule” are nucleic acid compounds which are capable of reducing the expression of a gene or the activity of the product of such gene to an extent sufficient to achieve a desired biological or physiological effect. The term “siNA inhibitor” as used herein refers to one or more of a siRNA, shRNA, synthetic shRNA; miRNA. Inhibition may also be referred to as down-regulation or, for RNAi, silencing. The dsRNA molecule includes a sense strand, also known as a passenger strand, which shares homology to a target RNA; and an antisense strand, also known as a guide strand, which is fully or partially complementary to the sense strand.

“Modulate gene expression” includes down regulating (e.g. siRNA) gene expression or up regulating (e.g. saRNA) activity of target coding or non-coding RNA or protein or genomic DNA. As used herein, the term “inhibition” of a target gene or “down regulation of gene expression” means inhibition of gene expression (transcription or translation) or polypeptide activity. The polynucleotide sequence of the target RNA sequence, refers to a mRNA target, a RNA target or any homologous sequences thereof preferably having at least 70% identity, more preferably 80% identity, even more preferably 90% or 95% identity to the target mRNA or RNA. Therefore, polynucleotide sequences, which have undergone mutations, alterations or modifications as described herein are encompassed in the present invention. The terms “mRNA polynucleotide sequence” and “mRNA” are used interchangeably. Throughout this disclosure, mRNA sequences are set forth as representing the corresponding genes.

As used herein, the terms “polynucleotide” and “nucleic acid” may be used interchangeably and refer to nucleotide sequences comprising deoxyribonucleic acid (DNA) and/or ribonucleic acid (RNA) and/or modified nucleotides and/or unconventional moieties. The terms DNA and RNA are to be understood to include, as equivalents, analogs of either DNA or RNA made from nucleotide analogs and or unconventional moieties.

“Oligonucleotide” or “oligomer” refers to a nucleic acid sequences from about 2 to about 100 nucleotides. Each oligonucleotide may be independently natural or synthetic, and or modified or unmodified. Modifications include changes to the sugar moiety, the base moiety and or the linkages between nucleotides in the oligonucleotide. The dsRNA molecules disclosed herein may comprise deoxyribonucleotides, ribonucleotides, modified deoxyribonucleotides, modified ribonucleotides, nucleotide analogs, modified nucleotide analogs, unconventional and abasic moieties and combinations thereof

A “conjugate” refers to a compound formed by the union of two compounds via covalent bonding. For example a sphingolipid-polyalkylamine moiety is a conjugate between a sphingolipid and a polyalkylamine. A sphingolipid-polyalkylamine oligonucleotide compound refers to a conjugate between an oligonucleotide (ssNA, dsNA etc) and a sphingolipid-polyalkylamine moiety. Various methods of synthesis are described below, in the Examples. The oligonucleotide may be directly attached to the sphingolipid polyalkylamine moiety or me be attached via a linker.

As used herein, “linker” and “linkage” refer to one or more atoms that join one chemical moiety to another chemical moiety, for example the sphingolipid-polyalkylamine to the phosphoramidite or NHS ester or the sphingolipid-polyalkylamine to the oligonucleotide. The linker is a nucleotide or non-nucleotide agent comprising one atom or a chain of for example, 2, 3, 4, 5, 6, 7, 8, 9, 10, 11, 12, 13, 14, 15, 16, 17, 18, 19, or 20 atoms including carbon, oxygen, sulfur, nitrogen and phosphorus atoms or combinations thereof. Examples of linkers include relatively low molecular weight groups such as alkyl, hydrocarbonyl, amide, ester, carbonate and ether, as well as higher molecular weight linking groups such as polyethylene glycol (PEG) as well as alkyl chains.

As used herein, the term “duplex region” refers to the region in the double stranded molecule in which two complementary or substantially complementary oligonucleotides form base pairs with one another, typically by Watson-Crick base pairing or by any other manner that allows for a duplex formation. The length of the RNA duplex is from about 15 to about 49 ribonucleotides, or about, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49, preferably 18-40, 18-27, 18-25 or 19 to 23 ribonucleotides. In some embodiments the length of each strand (oligomer) is independently selected from the group consisting of about 18 to about 40 nucleotides, preferably 18 to 27, 18 to 25, 19-23 and more preferably 19 ribonucleotides. For example, an oligonucleotide strand having 19, 20, 21, 22 nucleotide units can base pair with a complementary oligonucleotide of 19, 20, 21, 22 nucleotide units, or can base pair with 15, 16 17 or 18 nucleotides on each strand such that the “duplex region” consists of 15, 16 17 or 18 base pairs. The remaining base pairs may, for example, exist as 5′ and 3′ overhangs. Further, within the duplex region, 100% complementarity is not required; substantial complementarity is allowable within a duplex region. The overhang region may consist of nucleotide or non-nucleotide moieties. As disclosed herein at least one overhang region consists of one or more non-nucleotide moieties.

As used herein, the term “halogen” includes fluoro, chloro, bromo, and iodo, and is preferably fluoro, chloro or bromo.

The term “hydrocarbyl” in the definition of R⁶ refers to a radical containing only carbon and hydrogen atoms that may be saturated or unsaturated, linear or branched, cyclic or acyclic, or aromatic, and includes (C₁-C₈)alkyl, (C₂-C₈)alkenyl, (C₂-C₈)alkynyl, (C₃-C₁₀)cycloalkyl, (C₃-C₁₀)cycloalkenyl, (C₆-C₁₄)aryl, (C₁-C₈)alkyl(C₆-C₁₄)aryl, and (C₆-C₁₄)aryl(C₁-C₈)alkyl.

The term “(C₁-C₂₄) alkyl” typically means a straight or branched hydrocarbon radical having 1-24 carbon atoms and includes, e.g., methyl, ethyl, n-propyl, isopropyl, n-butyl, sec-butyl, isobutyl, tert-butyl, n-pentyl, 2,2-dimethylpropyl, n-hexyl, n-heptyl, n-octyl, and the like. Depending on the residue, preferred are (C₁-C₄)alkyl groups, most preferably methyl and ethyl or “(C₇-C₂₄) alkyl”. The terms “(C₂-C₈)alkenyl” and “(C₂-C₈)alkynyl” typically mean straight and branched hydrocarbon radicals having 2-8 carbon atoms and 1 double or triple bond, respectively, and include ethenyl, 3-buten-1-yl, 2-ethenylbutyl, 3-octen-1-yl, and the like, and propynyl, 2-butyn-1-yl, 3-pentyn-1-yl, and the like. (C₂-C₆)alkenyl and alkynyl radicals are preferred, more preferably (C₂-C₄)alkenyl and alkynyl.

The term “(C₁-C₈)alkylene” typically means a divalent straight or branched hydrocarbon radical having 1-8 carbon atoms and includes, e.g., methylene, ethylene, propylene, butylene, 2-methylpropylene, pentylene, 2-methylbutylene, hexylene, 2-methylpentylene, 3-methylpentylene, 2,3-dimethylbutylene, heptylene, octylene, and the like. Preferred are (C₁-C₄)alkylene, more preferably (C₁-C₂)alkylene.

The term “phosphate moiety” as used herein refers to a monophosphate moiety of the general formula —[O—P(O)(R′)—O]²⁻, a diphosphate moiety of the general formula —[O—P(O)(R′)—O—P(O)(R′)—O]³⁻, or a triphosphate moiety of the general formula —[O—P(O)(R′)—O—P(O)(R′)—O—P(O)(R′)—O]⁴⁻, wherein R′ each independently is O⁻, S⁻, BH₃⁻, or N⁻, preferably to such mono-, di- and tri-phosphate moieties wherein (i) R′ each is O⁻; or (ii) one of the R's, preferably the R′ linked to the phosphate atom at position α, is S⁻ or BH₃⁻, and the other R's are O⁻, as well as to any protonated form thereof. Preferred are monophosphate moieties as defined above, such as —[O—PO₃]²⁻, —[O—PO₂S]²⁻, and [O—PO₂(BH₃)]²⁻, more preferably —[O—PO₃]²⁻.

The term “phosphate linking moiety” as used herein refers to a moiety of the general formula —[O—P(O)(R′)]⁻—, wherein R′ is O⁻, S⁻, BH₃⁻, or N⁻, preferably O⁻, S⁻, or BH₃⁻, more preferably O⁻, as well as to a protonated form thereof

“Terminal functional group” includes halogen, alcohol, amine, carboxylic, ester, amide, aldehyde, ketone, ether groups.

According to one aspect provided herein are compounds comprising chemically modified dsRNA molecules comprising unmodified ribonucleotides, modified ribonucleotides and/or unconventional moieties covalently linked to least one sphingolipid-polyalkylamine conjugate. In some embodiments the chemically modified dsRNA comprises at least one modified nucleotide selected from the group consisting of a sugar modification, a base modification and an internucleotide linkage modification and may contain one or more unconventional moiety DNA, TNA (threose nucleic acid), LNA (locked nucleic acid), ENA (ethylene-bridged nucleic acid), L-DNA or L-RNA, PNA (peptide nucleic acid), arabinoside, phosphonocarboxylate or phosphinocarboxylate nucleotide (PACE nucleotide), or nucleotides with a 6 carbon sugar. All analogs of, or modifications to, a nucleotide/oligonucleotide are employed with the molecules described herein, provided that said analog or modification does not substantially adversely affect the properties, e.g. function, of the oligonucleotide.

In some embodiments a modified ribonucleotide is a 2′OMe (2′ methoxy) sugar modified ribonucleotide. In some embodiments some or all of the pyrimidine ribonucleotides in the antisense strand comprise 2′OMe sugar modified ribonucleotides. In some embodiments some or all of the purines in the antisense strand comprise 2′OMe sugar modified ribonucleotides. In preferred embodiments the antisense strand comprises 2′OMe sugar modified ribonucleotides in nuclease sensitive positions. In some embodiments the sense strand comprises 2′OMe sugar modified ribonucleotides in nuclease sensitive positions. In some embodiments the sense strand [e.g. (N′)y or N2′(N′)y] comprises one or more 2′OMe sugar modified ribonucleotides. In some embodiments the sense strand comprises one or more deoxyribonucleotide. In some embodiments the siRNA is blunt ended at the 3′ terminus of the compound, i.e. the dsRNA or siRNA is blunt ended on the end defined by the 3′-terminus of the sense or passenger strand and the 5′-terminus of antisense or guide strand. In some embodiments the 3′terminus comprises a 3′Pi (3′ terminal phosphate). In some embodiments the 5′terminus comprises a 5′Pi (5′ terminal phosphate).

In some embodiments nucleotides are selected from those having naturally occurring or synthetic modified bases. Naturally occurring bases include adenine, guanine, cytosine, thymine and uracil. Modified bases of nucleotides include pyrazolotriazine, inosine, xanthine, hypoxanthine, 2-aminoadenine, 6-methyl, 2-propyl and other alkyl adenines, 5-halouracil, 5-halocytosine, 6-azacytosine and 6-az thymine, pseudouracil, deoxypseudouracil, 4-thiouracil, ribo-2-thiouridine, ribo-4-thiouridine, 8-haloadenine, 8-aminoadenine, 8-thioladenine, 8-thiolalkyl adenines, 8-hydroxyl adenine and other 8-substituted adenines, 8-haloguanines, 8-aminoguanine, 8-thiolguanine, 8-thioalkylguanines 8-hydroxylguanine and other substituted guanines, other aza and deaza adenines, other aza and deaza guanines, 5-methylribouridine, 5-trifluoromethyl uracil, 5-methylribocytosine, and 5-trifluorocytosine. In some embodiments one or more nucleotides in an oligomer is substituted with inosine.

Modified deoxyribonucleotide includes, for example 5′OMe DNA (5-methyl-deoxyriboguanosine-3′-phosphate); PACE (deoxyriboadenosine 3′ phosphonoacetate, deoxyribocytidine 3′ phosphonoacetate, deoxyriboguanosine 3′ phosphonoacetate, deoxyribothymidine 3′ phosphonoacetate).

Bridged nucleic acids include LNA (2′-O, 4′-C-methylene bridged Nucleic Acid adenosine 3′ monophosphate, 2′-O,4′-C-methylene bridged Nucleic Acid 5-methyl-cytidine 3′ monophosphate, 2′-O,4′-C-methylene bridged Nucleic Acid guanosine 3′ monophosphate, 5-methyl-uridine (or thymidine) 3′ monophosphate); and ENA (2′-O,4′-C-ethylene bridged Nucleic Acid adenosine 3′ monophosphate, 2′-O,4′-C-ethylene bridged Nucleic Acid 5-methyl-cytidine 3′ monophosphate, 2′-O,4′-C-ethylene bridged Nucleic Acid guanosine 3′ monophosphate, 5-methyl-uridine (or thymidine) 3′ monophosphate).

A sugar modification includes a modification on the 2′ moiety of the sugar residue and encompasses amino, fluoro, alkoxy (e.g. methoxy), alkyl, amino, fluoro, chloro, bromo, CN, CF, imidazole, carboxylate, thioate, C₁-C₁₀ lower alkyl, substituted lower alkyl, alkaryl or aralkyl, OCF₃, OCN, O-, S-, or N-alkyl; O-, S-, or N-alkenyl; SOCH₃; SO₂CH₃; ONO₂; NO₂, N₃; heterocycloalkyl; heterocycloalkaryl; aminoalkylamino; polyalkylamino or substituted silyl, as, among others, described in European patents EP 0 586 520 B1 or EP 0 618 925 B1.

In one embodiment the modified molecules comprise at least one ribonucleotide comprising a 2′ modification on the sugar moiety (“2′ sugar modification”). In certain embodiments the sugar modified moiety comprises 2′O-alkyl or 2′-fluoro or 2′O-allyl or any other 2′ modification. In some embodiments a preferred 2′O-alkyl is 2′O-methyl (methoxy) sugar modification. Other stabilizing modifications are also possible (e.g. terminal modifications).

In some embodiments the backbone of the oligonucleotides is modified and comprises phosphate-D-ribose entities but may also contain thiophosphate-D-ribose entities, triester, thioate, 2′-5′ bridged backbone (also may be referred to as 2′5′ linked nucleotide or 5′-2′), PACE and the like. Additional modifications include reversible or labile phosphotriester linkages such as those disclosed in US2009093425 and US2011294869, respectively.

As used herein, the terms “non-pairing nucleotide analog” means a nucleotide analog which comprises a non-base pairing moiety including but not limited to: 6 des amino adenosine (Nebularine), 4-Me-indole, 3-nitropyrrole, 5-nitroindole, Ds, Pa, N3-Me riboU, N3-Me riboT, N3-Me dC, N3-Me-dT, N1-Me-dG, N1-Me-dA, N3-ethyl-dC, N3-Me dC. In some embodiments the non-base pairing nucleotide analog is a ribonucleotide (2′OH). In other embodiments the non-base pairing nucleotide analog is a deoxyribonucleotide (2′H). In addition, analogs of polynucleotides may be prepared wherein the structure of one or more nucleotide is fundamentally altered and better suited as therapeutic or experimental reagents. An example of a nucleotide analog is a peptide nucleic acid (PNA) wherein the deoxyribose (or ribose) phosphate backbone in DNA (or RNA) is replaced with a polyamide backbone which is similar to that found in peptides. PNA analogs have been shown to be resistant to enzymatic degradation and to have enhanced stability in vivo and in vitro. Other modifications include polymer backbones, cyclic backbones, acyclic backbones, thiophosphate-D-ribose backbones, triester backbones, thioate backbones, 2′-5′ bridged backbone, artificial nucleic acids, morpholino nucleic acids, glycol nucleic acid (GNA), threose nucleic acid (TNA), arabinoside, and mirror nucleoside (for example, beta-L-deoxyribonucleoside instead of beta-D-deoxyribonucleoside). Examples of siRNA compounds comprising LNA nucleotides are disclosed in Elmen et al., (NAR 2005, 33(1):439-447).

Other modifications include 3′ terminal modifications also known as capping moieties. Such terminal modifications are selected from a nucleotide, a modified nucleotide, a lipid, a peptide, a sugar and inverted abasic moiety. Such modifications are incorporated, for example at the 3′ terminus of the sense and/or antisense strands.

The term “capping moiety” as used herein includes abasic ribose moiety, abasic deoxyribose moiety, modifications abasic ribose and abasic deoxyribose moieties including 2′ O alkyl modifications; inverted abasic ribose and abasic deoxyribose moieties and modifications thereof; C6-imino-Pi; a mirror nucleotide including L-DNA and L-RNA; 5′O-Me nucleotide; and nucleotide analogs including 4′,5′-methylene nucleotide; 1-(β-D-erythrofuranosyl)nucleotide; 4′-thionucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non bridging methylphosphonate and 5′-mercapto moieties.

Certain preferred capping moieties are abasic ribose or abasic deoxyribose moieties; inverted abasic ribose or abasic deoxyribose moieties; C6-amino-Pi; a mirror nucleotide including L-DNA and L-RNA. In some embodiments the molecules are synthesized with one or more inverted nucleotides, for example inverted thymidine or inverted adenosine (see, for example, Takei, et al., 2002, JBC 277(26):23800-06). In some embodiments an inverted abasic deoxyribose moiety is covalently attached to the 5′ terminus of the sense strand (N′)y.

“Terminal functional group” includes halogen, alcohol, amine, carboxylic, ester, amide, aldehyde, ketone, ether groups.

The term “unconventional moiety” as used herein refers to abasic ribose moiety, an abasic deoxyribose moiety, a deoxyribonucleotide, a modified deoxyribonucleotide, a mirror nucleotide, a non-base pairing nucleotide analog and a nucleotide joined to an adjacent nucleotide by a 2′-5′ internucleotide phosphate bond; a pyrazolotriazine nucleotide analog; a threose nucleic acid (TNA) moiety; unlocked nucleic acids (UNA), bridged nucleic acids including locked nucleic acids (LNA) and ethylene bridged nucleic acids (ENA) and morpholinos.

“TNA” refers to (L)-alpha-threofuranosyl nucleotides. The TNA phosphoramidites are linked to adjacent TNA, deoxyribonucleotide or ribonucleotide by (3′->2′) phosphodiester linkages. TNA comprise a four-carbon sugar (Schoning, et al Science 2000. 290:1347-51). In some embodiments, in addition to TNA the siRNA compound further comprises at least one modified ribonucleotide selected from the group consisting of a ribonucleotide having a sugar modification, a base modification or an internucleotide linkage modification and may contain DNA, a mirror nucleotide (L-DNA, L-RNA) and modified nucleotides such as LNA (locked nucleic acid), ENA (ethylene-bridged nucleic acid), PNA (peptide nucleic acid), arabinoside, phosphonocarboxylate or phosphinocarboxylate nucleotide (PACE nucleotide), or nucleotides with a 6 carbon sugar.

What is sometimes referred to herein as an “abasic nucleotide” or “abasic nucleotide analog” is more properly referred to as a pseudo-nucleotide or an unconventional moiety. A nucleotide is a monomeric unit of nucleic acid, consisting of a ribose or deoxyribose sugar, a phosphate, and a base (adenine, guanine, thymine, or cytosine in DNA; adenine, guanine, uracil, or cytosine in RNA). A modified nucleotide comprises a modification in one or more of the sugar, phosphate and or base. The abasic pseudo-nucleotide lacks a base, and thus is not strictly a nucleotide. Abasic deoxyribose moiety includes for example abasic deoxyribose-3′-phosphate; 1,2-dideoxy-D-ribofuranose-3-phosphate; 1,4-anhydro-2-deoxy-D-ribitol-3-phosphate. Inverted abasic deoxyribose moiety includes inverted deoxyriboabasic; 3′,5′ inverted deoxyabasic 5′-phosphate.

A “mirror” nucleotide is a nucleotide with reversed chirality to the naturally occurring or commonly employed nucleotide, i.e., a mirror image (L-nucleotide) of the naturally occurring (D-nucleotide), also referred to as L-RNA in the case of a mirror ribonucleotide, and “spiegelmer”. The mirror nucleotide is a ribonucleotide or a deoxyribonucleotide and my further comprise at least one sugar, base and or backbone modification. See U.S. Pat. No. 6,586,238. Also, U.S. Pat. No. 6,602,858 discloses nucleic acid catalysts comprising at least one L-nucleotide substitution. Mirror nucleotide includes for example L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror dT) and L-RNA (L-riboadenosine-3′-phosphate (mirror rA); L-ribocytidine-3′-phosphate (mirror rC); L-riboguanosine-3′-phosphate (mirror rG); L-ribouridine-3′-phosphate (mirror dU).

In some embodiments a modified ribonucleotide is a 2′OMe sugar modified ribonucleotide. In some embodiments some or all of the pyrimidine ribonucleotides in the antisense strand comprise 2′OMe sugar modified ribonucleotides. In some embodiments some or all of the purines in the antisense strand comprise 2′OMe sugar modified ribonucleotides. In preferred embodiments the antisense strand comprises 2′OMe sugar modified ribonucleotides in nuclease sensitive positions. In some embodiments the sense strand comprises 2′OMe sugar modified ribonucleotides in nuclease sensitive positions. In some embodiments the sense strand [e.g. (N′)y] comprises one or more 2′OMe sugar modified ribonucleotides. In some embodiments the sense strand comprises one or more deoxyribonucleotide. In some embodiments the siRNA is blunt ended at the 3′ terminus of the compound, i.e. the dsRNA or siRNA is blunt ended on the end defined by the 3′-terminus of the sense or passenger strand and the 5′-terminus of antisense or guide strand.

In other embodiments at least one of the two strands has a 3′ overhang of at least one nucleotide at the 3′-terminus; the overhang comprises at least one deoxyribonucleotide. At least one of the strands optionally comprises an overhang of at least one nucleotide at the 3′-terminus. The overhang consists of from about 1 to about 5 nucleotides.

In other embodiments at least one of the two strands has a 3′ non-nucleotide overhang covalently attached at the 3′-terminus of the strand. In various embodiments the overhangs are independently selected from a nucleotide, a non-nucleotide and a combination thereof. In certain embodiments, each overhang, if present, is independently selected from a ribonucleotide, deoxyribonucleotide, abasic deoxyribose moiety, abasic deoxyribose moiety, C3-amino-Pi, C4-amino-Pi, C5-amino-Pi, C6-amino-Pi, a mirror nucleotide.

In some embodiments each of Z and/or Z′ independently includes a C2, C3, C4, C5 or C6 alkyl moiety, optionally a C3 [propane, —(CH2)₃—] moiety or a derivative thereof including propanol (C3-OH), propanediol, and phosphodiester derivative of propanediol (“C3Pi”). In preferred embodiments each of Z and/or Z′ includes two hydrocarbon moieties and in some examples is C3Pi-C3OH or C3Pi-C3Pi. Each C3 is covalently conjugated to an adjacent C3 via a covalent bond, preferably a phospho-based bond. In some embodiments the phospho-based bond is a phosphorothioate, a phosphonoacetate or a phosphodiester bond.

In a specific embodiment x=y=19 and Z comprises C3-C3. In some embodiments the C3-C3 overhang is covalently attached to the 3′ terminus of (N)x or (N′)y via a covalent linkage, for example a phosphodiester linkage. In some embodiments the linkage between a first C3 and a second C3 is a phosphodiester linkage. In some embodiments the 3′ non-nucleotide overhang is C3Pi-C3Pi. In some embodiments the 3′ non-nucleotide overhang is C3Pi-C3Ps. In some embodiments the 3′ non-nucleotide overhang is C3Pi-C3OH (OH is hydroxy). In some embodiments the 3′ non-nucleotide overhang is C3Pi-C3OH.

In various embodiments the alkyl moiety comprises an alkyl derivative including a C3 alkyl, C4 alkyl, C5 alky or C6 alkyl moiety comprising a terminal hydroxyl, a terminal amino, or terminal phosphate group. In some embodiments the alkyl moiety is a C3 alkyl or C3 alkyl derivative moiety. In some embodiments the C3 alkyl moiety comprises propanol, propylphosphate, propylphosphorothioate or a combination thereof

The C3 alkyl moiety is covalently linked to the 3′ terminus of (N′)y and/or the 3′ terminus of (N)x via a phosphodiester bond. In some embodiments the alkyl moiety comprises propanol, propyl phosphate or propyl phosphorothioate.

The structures of exemplary 3′ terminal C3 non-nucleotide moieties are as follows:

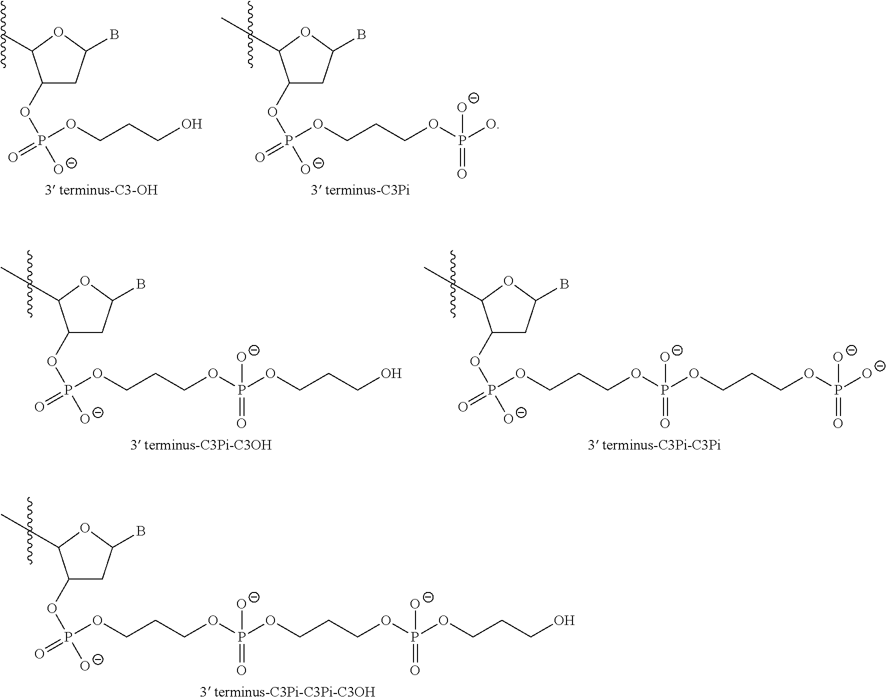

In some embodiments each of Z and Z′ is independently selected from propanol, propyl phosphate propyl phosphorothioate, combinations thereof or multiples thereof in particular 2 or 3 covalently linked propanol, propyl phosphate, propyl phosphorothioate or combinations thereof. In some embodiments, when the 3′ terminal nucleotide comprises a 2′5′ linked nucleotide the C3 moiety may be linked to the 2′ position of the sugar via a phosphodiester linkage or other linkage.

In some embodiments each of Z and Z′ is independently selected from propyl phosphate, propyl phosphorothioate, propyl phospho-propanol; propyl phospho-propyl phosphorothioate; propylphospho-propyl phosphate; (propyl phosphate)₃, (propyl phosphate)₂-propanol, (propyl phosphate)₂-propyl phosphorothioate. Any propane or propanol conjugated moiety can be included in Z or Z′.

In additional embodiments each of Z and/or Z′ comprises a combination of an abasic moiety and an unmodified deoxyribonucleotide or ribonucleotide or a combination of a hydrocarbon moiety and an unmodified deoxyribonucleotide or ribonucleotide or a combination of an abasic moiety (deoxyribo or ribo) and a hydrocarbon moiety. In such embodiments, each of Z and/or Z′ comprises C3Pi-rAb or C3Pi-dAb.

In some embodiments, the complementarity between the antisense strand of the dsRNA and the target nucleic acid is perfect. In other embodiments, the antisense strand of the modified siRNA compound and the target nucleic acid are substantially complementary, i.e. having one, two or up to three mismatches between said antisense strand and the target nucleic acid. In some embodiments the antisense strand is mismatched to the target mRNA at the 5′ terminal nucleotide.

In some embodiments, the complementarity between the antisense strand of the dsRNA and the target nucleic acid is perfect. In other embodiments, the antisense strand of the modified siRNA compound and the target nucleic acid are substantially complementary, i.e. having one, two or up to three mismatches between said antisense strand and the target nucleic acid. In some embodiments the antisense strand is mismatched to the target mRNA at the 5′ terminal nucleotide.

In certain embodiments the complementarity between the antisense strand and the sense strand of the dsRNA molecule is perfect. In some embodiments, the strands are substantially complementary, i.e. having one, two or up to three mismatches between said antisense strand and said sense strand. In some embodiments the antisense strand is fully complementary to the sense strand.

### RNAi Oligonucleotides

The presence of long dsRNAs in cells stimulates the activity of a ribonuclease III enzyme referred to as “dicer” (Bass, 2000, Cell, 101, 235; Zamore et al., 2000, Cell, 101, 25-33; Hammond et al., 2000, Nature, 404, 293). Dicer is involved in the processing of the dsRNA into short dsRNA pieces known as siNA or siRNA (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and include about 19 base pair duplexes (Zamore et al., 2000, Cell, 101, 25-33; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

RNAi has been studied in a variety of systems. Fire et al., 1998, Nature, 391, 806, were the first to observe RNAi in C. elegans. Bahramian and Zarbl, 1999, Molecular and Cellular Biology, 19, 274-283 and Wianny and Goetz, 1999, Nature Cell Biol., 2, 70, describe RNAi mediated by dsRNA in mammalian systems. Hammond et al., 2000, Nature, 404, 293, describe RNAi in Drosophila cells transfected with dsRNA. Elbashir et al., 2001, Nature, 411, 494 and Tuschl et al., International PCT Publication No. WO 01/75164, describe RNAi induced by introduction of duplexes of synthetic 21-nucleotide RNAs in cultured mammalian cells including human embryonic kidney and HeLa cells. Research in Drosophila embryonic lysates (Elbashir et al., 2001, EMBO J., 20, 6877 and Tuschl et al., International PCT Publication No. WO 01/75164) has revealed certain requirements for siRNA length, structure, chemical composition, and sequence that are essential to mediate efficient RNAi activity.

Nucleic acid molecules (for example having structural features as disclosed herein) may inhibit or down regulate gene expression or viral replication by mediating RNA interference “RNAi” or gene silencing in a sequence-specific manner; see e.g., Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Mello and Fire, International PCT Publication No. WO 01/29058; Li et al., International PCT Publication No. WO 00/44914; Hutvagner and Zamore, 2002, Science, 297, 2056-60; McManus et al., 2002, RNA, 8, 842-850.

The selection and synthesis of siRNA corresponding to known genes has been widely reported; (see for example Ui-Tei et al., J Biomed Biotech. 2006; 2006: 65052; Chalk et al., BBRC. 2004, 319(1): 264-74; Sioud & Leirdal, Met. Mol Biol.; 2004, 252:457-69; Levenkova et al., Bioinform. 2004, 20(3):430-2; Ui-Tei et al., NAR. 2004, 32(3):936-48; De Paula et al., RNA 2007, 13:431-56).

For examples of the use of, and production of, modified siRNA see, for example, Braasch et al., Biochem. 2003, 42(26):7967-75; Chiu et al., RNA, 2003, 9(9):1034-48; PCT publications WO 2004/015107 (atugen AG) and WO 02/44321 (Tuschl et al). U.S. Pat. Nos. 5,898,031 and 6,107,094, describe chemically modified oligomers. US Patent Publication Nos. 2005/0080246 and 2005/0042647 relate to oligomeric compounds having an alternating motif and nucleic acid molecules having chemically modified internucleoside linkages, respectively.

Other modifications have been disclosed. The inclusion of a 5′-phosphate moiety was shown to enhance activity of siRNAs in Drosophila embryos (Boutla, et al., Curr. Biol. 2001, 11:1776-1780) and is required for siRNA function in human HeLa cells (Schwarz et al., Mol. Cell, 2002, 10:537-48). Amarzguioui et al., (NAR, 2003, 31(2):589-95) showed that siRNA activity depended on the positioning of the 2′-O-methyl modifications. Holen et al (NAR. 2003, 31(9):2401-07) report that an siRNA having small numbers of 2′-O-methyl modified nucleosides gave good activity compared to wild type but that the activity decreased as the numbers of 2′-O-methyl modified nucleosides was increased. Chiu and Rana (RNA. 2003, 9:1034-48) describe that incorporation of 2′-O-methyl modified nucleosides in the sense or antisense strand (fully modified strands) severely reduced siRNA activity relative to unmodified siRNA. The placement of a 2′-O-methyl group at the 5′-terminus on the antisense strand was reported to severely limit activity whereas placement at the 3′-terminus of the antisense and at both termini of the sense strand was tolerated (Czauderna et al., NAR. 2003, 31(11):2705-16; WO 2004/015107). The molecules of the disclosed herein offer an advantage in that they are stable and active and are useful in the preparation of pharmaceutical compositions for treatment of various diseases.

PCT Patent Publication Nos. WO 2008/104978, WO 2009/044392, WO 2011/066475 and WO 2011/084193 to a co-assignee of the present invention and hereby incorporated by reference in their entirety, disclose dsRNA structures.

PCT Publication No. WO 2008/050329 and U.S. Ser. No. 11/978,089 to a co-assignee of the present invention relate to inhibitors of pro-apoptotic genes, and are incorporated by reference in their entirety.

PCT Patent Publication Nos. WO 2004/111191 and WO 2005/001043 relate to methods for enhancing RNAi.

The role of microRNAs in various diseases is being actively researched and novel targets for gene modulation are continuously being identified.

Provided herein is a method of modulating the expression of target gene in a cell by at least 20%, 30%, 40% or 50% as compared to a control, comprising contacting a cell with one or more of the compounds of the invention.

Additionally provided herein is a method of modulating the expression of target gene in a mammal by at least 20%, 30%, 40% or 50% as compared to a control, comprising administering one or more of the dsRNA molecules disclosed herein to the mammal. In a preferred embodiment the mammal is a human.

Modulating gene expression is down-regulating gene expression or up-regulating gene expression.

In various embodiments the down-regulation of the expression of a target gene is selected from the group comprising down-regulation of gene function (which is examined, e.g. by an enzymatic assay or a binding assay with a known interactor of the native gene/polypeptide, inter alia), down-regulation of polypeptide product of the gene (which is examined, e.g. by Western blotting, ELISA or immuno-precipitation, inter alia) and down-regulation of mRNA expression of the gene (which is examined, e.g. by Northern blotting, quantitative RT-PCR, in-situ hybridization or microarray hybridization, inter alia).

In other embodiments modulation is up-regulation and the up-regulation of the expression of a target gene is selected from the group comprising up-regulation of gene function (which is examined, e.g. by an enzymatic assay or a binding assay with a known interactor of the native gene/polypeptide, inter alia), up-regulation of polypeptide product of the gene (which is examined, e.g. by Western blotting, ELISA or immuno-precipitation, inter alia) and up-regulation of mRNA expression of the gene (which is examined, e.g. by Northern blotting, quantitative RT-PCR, in-situ hybridization or microarray hybridization, inter alia).

In preferred embodiments the oligonucleotide useful for conjugation to the sphingolipid-polyalkylamine is a RNA interference (RNAi) oligonucleotide. A RNAi oligonucleotide is a molecule capable of inducing RNA interference through interaction with the RNA interference pathway machinery of mammalian cells to degrade or inhibit translation of messenger RNA (mRNA) transcripts of a transgene in a sequence specific manner. Two primary RNAi oligonucleotide are small (or short) interfering RNAs (siRNA) and micro RNAs (miRNA or miR). RNAi oligonucleotides may be for example RNA antisense, siRNA, siNA, miRNA, double-strand RNA (dsRNA), short hairpin RNA (shRNA). RNAi oligonucleotides may be chemically synthesized using standard synthesizers or recombinantly synthesized using expression cassettes encoding RNA capable of inducing RNAi. In some embodiments the oligonucleotide is a single-stranded oligonucleotide or a double-stranded oligonucleotide. Single-stranded oligonucleotides include antisense molecules (DNA, RNA or DNA/RNA chimeras) and miRNA mimetics. Double-stranded oligonucleotides include siRNA, siNA, shRNA and miRNA.

RNAi oligonucleotides may be chemically synthesized using standard synthesizers or recombinantly synthesized using expression cassettes encoding RNA capable of inducing RNAi. RNAi polynucleotide expression cassettes can be transcribed in the cell to produce small hairpin RNAs that can function as siRNA, separate sense and anti-sense strand linear siRNAs, or miRNA. RNA polymerase III transcribed DNAs contain promoters selected from the list comprising: U6 promoters, H1 promoters, and tRNA promoters. RNA polymerase II promoters include U1, U2, U4, and U5 promoters, snRNA promoters, microRNA promoters, and mRNA promoters.

siRNA comprises a double stranded structure typically containing 15-49 base pairs and preferably 18-25 base pairs and having a nucleotide sequence identical (perfectly complementary) or nearly identical (partially complementary) to a coding sequence in an expressed target gene or RNA within the cell. A siRNA may have dinucleotide 3′ overhangs. A siRNA may be composed of two annealed polynucleotides or a single polynucleotide that forms a hairpin structure. A siRNA molecule of the invention comprises a sense region and an antisense region. In one embodiment, the siRNA of the conjugate is assembled from two oligonucleotide fragments wherein one fragment comprises the nucleotide sequence of the antisense strand of the siRNA molecule and a second fragment comprises nucleotide sequence of the sense region of the siRNA molecule. In another embodiment, the sense strand is connected to the antisense strand via a linker molecule, such as a polynucleotide linker or a non-nucleotide linker. MicroRNAs (miRNAs) are small noncoding RNA gene products about 22 nucleotides long that direct destruction or translational repression of their mRNA targets. If the complementarity between the miRNA and the target mRNA is partial, translation of the target mRNA is repressed. If complementarity is extensive, the target mRNA is cleaved. For miRNAs, the complex binds to target sites usually located in the 3′ UTR of mRNAs that typically share only partial homology with the miRNA. A “seed region”—a stretch of about seven (7) consecutive nucleotides on the 5′ end of the miRNA that forms perfect base pairing with its target—plays a key role in miRNA specificity. Binding of the RISC/miRNA complex to the mRNA can lead to either the repression of protein translation or cleavage and degradation of the mRNA. Recent data indicate that mRNA cleavage happens preferentially if there is perfect homology along the whole length of the miRNA and its target instead of showing perfect base-pairing only in the seed region (Pillai et al. 2007).

Generic non-limiting nucleic acid molecule patterns are shown below where N′=sense strand nucleotide in the duplex region; z″=5′-capping moiety covalently attached at the 5′ terminus of the sense strand; C3=3 carbon non-nucleotide moiety; N=antisense strand nucleotide in the duplex region; idB=inverted abasic deoxyribonucleotide non-nucleotide moiety. Each N, N′, is independently modified or unmodified or an unconventional moiety. The sense and antisense strands are each independently 18-40 nucleotides in length. The examples provided below have a duplex region of 19 nucleotides; however, nucleic acid molecules disclosed herein can have a duplex region anywhere between 15 and 49 nucleotides, or between 18 and 40 nucleotides and where each strand is independently between 18 and 40 nucleotides, preferably 19-23 nucleotides (including modified nucleotides and or unconventional moieties) in length. In each duplex the antisense strand (N)x is shown on top (5′>3′) and the sense strand below (3′>5′). “SL” refers to a sphingolipid-polyalkylamine conjugate. Non-limiting examples of sphingolipid-polyalkylamine-dsRNA molecule have the following structure:

5′ (N)₁₉

3′ SL-(N′)₁₉

5′ (N)₁₉

3′ (N′)₁₉-SL

5′ (N)₁₉-SL

3′ (N′)₁₉

5′ (N)₁₉—C3Pi-C3Pi

3′ (N′)₁₉-SL

5′ (N)₁₉—C3Pi-C3Pi

3′ PiC3-(N′)₁₉-SL

5′ (N)₁₉-dTdT

3′ PiC3-(N′)₁₉-SL

5′ (N)₁₉-dTdT

3′ dTdT-(N′)₁₉-SL

5′ (N)₁₉—C3Pi-C3Pi

3′ dTdT-(N′)₁₉-SL

5′ (N)₁₉-dTdT-SL

3′ PiC3-(N′)₁₉-SL

5′ (N)₁₉—C3Pi-C3Pi

3′ SL-(N′)₁₉-z″

5′ (N)₁₉—C3Pi-C3Pi

3′ HOC3-(N′)₁₉-SL

wherein each N and N′ is independently an unmodified ribonucleotide, a modified ribonucleotide or is an unconventional moiety;
wherein each N is linked to the adjacent N by a covalent bond;
wherein each N′ is linked to the adjacent N′ by a covalent bond; and
wherein SL is a sphingolipid-polyalkylamine conjugate covalently attached at a terminus; and
wherein C3OH, C3Pi and the like refer to C3 non-nucleotide moieties covalently attached at the 3′ termini of a strand;
wherein dTdT refers to a thymidine dinucleotide;
wherein z″ is a capping moiety covalently attached to the 5′ terminus of the sense strand.

In some embodiments, the dsRNA comprises Z, a sphingolipid-polyalkylamine covalently attached to the 5; terminus of the sense strand, and 3, 4, or 5 2′-5′ linked ribonucleotides present at the 3′ terminus of the sense strand. In additional embodiments the compound comprises a nucleotide joined to an adjacent nucleotide by a 2′-5′ internucleotide phosphate bond in position 6, 7, or 8 (5′>3′) of the antisense strand.

In additional, when x=y=19 and the nucleotides at positions 15-19 or 16-19 or 17-19 in (N′)y are joined to adjacent nucleotides by 2′-5′ internucleotide phosphate bonds. In some embodiments x=y=19 and the nucleotides at positions 15-19 or 16-19 or 17-19 or 15-18 or 16-18 in (N′)y are joined to the adjacent nucleotides by 2′-5′ internucleotide phosphate bonds.

In some embodiments (N)x comprises modified and unmodified ribonucleotides, each modified ribonucleotide having a 2′-O-methyl on its sugar, wherein N at the 3′ terminus of (N)x is a modified ribonucleotide, (N)x comprises at least five alternating modified ribonucleotides beginning at the 3′ end and at least nine modified ribonucleotides in total and each remaining N is an unmodified ribonucleotide. In some embodiments, (N)x further includes an unconventional moiety selected from TNA and 2′5′ linked nucleotide.

In some embodiments in (N′)y at least one unconventional moiety is present, which unconventional moiety may be an abasic ribose moiety, an abasic deoxyribose moiety, a modified or unmodified deoxyribonucleotide, a mirror nucleotide, and a nucleotide joined to an adjacent nucleotide by a 2′-5′ internucleotide phosphate bond, or any other unconventional moiety disclosed herein.

In some embodiments an unconventional moiety is an L-DNA mirror nucleotide; in additional embodiments at least one unconventional moiety is present at positions 15, 16, 17, or 18 in (N′)y. In some embodiments the mirror nucleotide is an L-DNA moiety. In some embodiments the L-DNA moiety is present at position 17, position 18 or positions 17 and 18.

In some embodiments (N)x comprises nine alternating modified ribonucleotides. In other embodiments (N)x comprises nine alternating modified ribonucleotides further comprising a 2′ modified nucleotide at position 2. In some embodiments (N)x comprises 2′OMe modified ribonucleotides at the odd numbered positions 1, 3, 5, 7, 9, 11, 13, 15, 17, 19. In other embodiments (N)x further comprises a 2′OMe modified ribonucleotide at one or both of positions 2 and 18. In yet other embodiments (N)x comprises 2′OMe modified ribonucleotides at positions 2, 4, 6, 8, 11, 13, 15, 17, 19. In some embodiments at least one pyrimidine nucleotide (i.e. pyrimidine ribonucleotide) in (N)x comprises a 2′OMe sugar modification. In some embodiments all pyrimidine nucleotides (i.e. pyrimidine ribonucleotide) in (N)x comprises a 2′OMe sugar modification. In some embodiments 2, 3, 4, 5, 6, 7, 8, 9, 10, 11, 12, 13, 14, or 15 pyrimidine nucleotides in N(x) comprise a 2′OMe sugar modification

In one embodiment of the dsRNA molecules (N′)y comprises at least two nucleotides at either or both the 5′ and 3′ termini of (N′)y are joined by a 2′-5′ phosphodiester bond. In certain embodiments x=y=19; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; and three nucleotides at the 3′ terminus of (N′)y are joined by two 2′-5′ phosphodiester bonds. In other embodiments, x=y=19; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; and four consecutive nucleotides at the 5′ terminus of (N′)y are joined by three 2′-5′ phosphodiester bonds. In a further embodiment, an additional nucleotide located in the middle position of (N)y may be modified with 2′-O-methyl on its sugar. In another embodiment, in (N)x the nucleotides alternate between 2′-O-methyl modified ribonucleotides and unmodified ribonucleotides, and in (N′)y four consecutive nucleotides at the 5′ terminus are joined by three 2′-5′ phosphodiester bonds and the 5′ terminal nucleotide or two or three consecutive nucleotides at the 5′ terminus comprise 3′-O-Me sugar modifications.

In certain embodiments, x=y=19 and in (N′)y the nucleotide in at least one position comprises a mirror nucleotide, a deoxyribonucleotide and a nucleotide joined to an adjacent nucleotide by a 2′-5′ internucleotide bond. In certain embodiments (N′)y comprises 2′-5′ internucleotide bonds at positions 16, 17, 18, 16-17, 17-18, or 16-18. In certain embodiments (N′)y comprises 2′-5′ internucleotide bonds at positions 16, 17, 18, 16-17, 17-18, or 16-18 and a 5′ terminal cap nucleotide.

In various embodiments when the sphingolipid-polyalkylamine is linked to the 3′ terminus of the sense strand or to the antisense strand, z″ is present and is selected from an abasic ribose moiety, a deoxyribose moiety; an inverted abasic ribose moiety, a deoxyribose moiety; C6-amino-Pi; and a mirror nucleotide.

In various embodiments the double-stranded nucleic acid comprises at least one of the following modifications:

a threose nucleic acid moiety, a 2′5′ linked nucleotide or a mirror nucleotide in at least one of positions 5, 6, 7, 8, or 9 from the 5′ terminus of the antisense strand [(N)x];

a threose nucleic acid moiety, a 2′5′ linked nucleotide or a pseudoUridine in at least one of positions 9 or 10 from the 5′ terminus of the sense strand [(N′)y];

1, 2, 3, 4, 5, 6, 7, 8, 9 or 10 threose nucleic acid moieties or 2′5′ linked nucleotides at the 3′ terminal or penultimate positions the sense strand [(N′)y].

In some embodiments, the sequence of (N′)y is fully complementary to the sequence of (N)x. In some embodiments (N)x comprises an antisense that is fully complementary to about 17 to about 24 consecutive nucleotides in a target RNA.

The chemical modifications described herein are useful with any oligonucleotide pair (sense and antisense strands) to a mammalian or non-mammalian gene. In some embodiments the mammalian gene is a human gene.

In another aspect the provided is a method of generating a sphingolipid-polyalkylamine-dsRNA compound consisting of a sphingolipid-polyalkylamine conjugate attached to a dsRNA having a sense strand and an antisense strand comprising the steps of

a) selecting a consecutive 15 to 49 nucleotide sequence in a target RNA and synthesizing an antisense strand comprising complementarity to the consecutive 15 to 49 nucleotide sequence of the target mRNA;

b) synthesizing a sense strand of 8 to 49 nucleotides having complementarity to the antisense strand;

c) wherein at least one terminus of the sense strand or antisense strand is linked to a sphingolipid-polyalkylamine conjugate;

d) annealing the antisense and sense strands; thereby generating sphingolipid-polyalkylamine-dsRNA compound.

In some embodiments step a) includes selecting a consecutive 18 to 25 nucleotide, or 18, 19, 20, 21, 22, 23, 24 or 25 nucleotide sequence in a target RNA in a target cell wherein the 3′ terminal nucleotide is other than adenosine.

In preferred embodiments the chemically modified ribonucleotides are positioned along the sense strand and or the antisense strand and introduce a desired property upon the double-stranded compound including increased resistance to nucleases.

In some embodiments, x=19, a sphingolipid-polyalkylamine conjugate is covalently attached at the 5′ terminus of the sense strand and (N)x comprises a TNA moiety in position 5, in position 6, in position 7, in position 8, in position 9, in positions 5-6, in positions 6-7, in positions 7-8, in positions 8-9, in positions 5-7, in positions 6-8, in positions 7-9, in positions 5-8, in positions 6-9 or in positions 5-9. In some embodiments, x=19, a sphingolipid-polyalkylamine conjugate is covalently attached at the 5′ terminus of the sense strand and (N)x comprises a 2′-5′ nucleotide in position 5, in position 6, in position 7, in position 8, in position 9, in positions 5-6, in positions 6-7, in positions 7-8, in positions 8-9, in positions 5-7, in positions 6-8, in positions 7-9, in positions 5-8, in positions 6-9 or in positions 5-9. In preferred embodiments (N)x comprises a 2′-5′ nucleotide in position 5, in position 7, in position 8, in position 9, in positions 6-7, in positions 7-8, or in positions 8-9.

In some embodiments, a sphingolipid-polyalkylamine conjugate is covalently attached at the 5′ terminus of the sense strand N′ in at least one of positions 9 or 10 from the 5′ terminus of (N′)y. In some embodiments, (N′)y comprises a threose nucleic acid (TNA) moiety in position 9, or in position 10 or in positions 9-10. In some embodiments, (N′)y comprises a sphingolipid-polyalkylamine conjugate covalently attached at the 5′ terminus and a 2′5′ linked nucleotide in position 9, or in position 10 or in positions 9-10. In some embodiments, (N′)y comprises a sphingolipid-polyalkylamine conjugate covalently attached at the 5′ terminus and a mirror nucleotide in position 9, or in position 10 or in positions 9-10.

In some embodiments, (N′)y comprises a sphingolipid-polyalkylamine conjugate covalently attached at the 5′ terminus and 2′5′ linked nucleotides at the 4 most, 5 most or 6 most 3′ terminal positions of (N′)y. Without wishing to be bound to theory, a double-stranded nucleic acid molecule in the compound having multiple 2′5′ linked nucleotides at the 3′ terminus of the sense (passenger) strand confers increased nuclease stability to the duplex and or reduced off target effect of the sense (passenger) strand.

In some embodiments, (N′)y comprises a sphingolipid-polyalkylamine conjugate covalently attached at the 5′ terminus and 2′5′ linked nucleotides in the four 3′-most terminal positions. In some embodiments the x=y=19 and (N′)y comprises a sphingolipid-polyalkylamine conjugate covalently attached at the 5′ terminus and 2′5′ linked nucleotides in positions 16, 17, 18 and 19.

In some embodiments (N′)y comprises a sphingolipid-polyalkylamine conjugate covalently attached at the 5′ terminus and 2′5′ linked nucleotides in the five 3′-most terminal positions. In some embodiments the x=y=19 and (N′)y comprises a sphingolipid-polyalkylamine conjugate covalently attached at the 5′ terminus and 2′5′ linked nucleotides in positions 15, 16, 17, 18 and 19.

In some embodiments (N′)y comprises a sphingolipid-polyalkylamine conjugate covalently attached at the 5′ terminus and 2′5′ linked nucleotides in the six 3′-most terminal positions. In some embodiments the x=y=19 and (N′)y comprises a sphingolipid-polyalkylamine conjugate covalently attached at the 5′ terminus and 2′5′ linked nucleotides in positions 14, 15, 16, 17, 18 and 19.

In some embodiments (N′)y comprises a sphingolipid-polyalkylamine conjugate covalently attached at the 5′ terminus and 2′5′ linked nucleotides in the six 3′-most terminal positions. In some embodiments the x=y=19 and N²—(N′)y comprises 2'S′ linked nucleotides in positions 14, 15, 16, 17, 18 and 19.

The compounds may further comprise combinations of the aforementioned modifications, and 2′OMe sugar modified ribonucleotides including 2′OMe sugar modified pyrimidine ribonucleotides and or purine ribonucleotides in the sense strand and or antisense strand. In certain embodiments (N)x and (N′)y are fully complementary. In other embodiments (N)x and (N′)y are substantially complementary. In certain embodiments (N)x is fully complementary to a target sequence. In other embodiments (N)x is substantially complementary to a target sequence.

In some embodiments, neither strand of the modified dsRNA molecules disclosed herein is phosphorylated at the 3′ and 5′ termini. In other embodiments the sense and antisense strands are phosphorylated at the 3′ termini. In yet another embodiment, the antisense strand is phosphorylated at the terminal 5′ termini position using cleavable or non-cleavable phosphate groups. In yet another embodiment, either or both antisense and sense strands are phosphorylated at the 3′ termini position using cleavable or non-cleavable phosphate groups.

Unless otherwise indicated, in preferred embodiments of the structures discussed herein the covalent bond between each consecutive N and N′ is a phosphodiester bond. In some embodiments at least one of the covalent bond is a phosphorothioate bond.

For all of the structures above, in some embodiments the oligonucleotide sequence of (N)x is fully complementary to the oligonucleotide sequence of (N′)y. In other embodiments the antisense and sense strands are substantially complementary. In certain embodiments (N)x is fully complementary to a mammalian mRNA, a plant RNA, fungal RNA or microbial RNA including bacterial and viral RNA. In other embodiments (N)x is substantially complementary to a mammalian mRNA. In some embodiments, the target of oligonucleotide compound is genomic DNA belonging to mammalian or viral genomes, preferably a human mRNA.

In some embodiments the dsRNA molecule is a siRNA, siNA or a miRNA.

Further provided is a pharmaceutical composition comprising a compound disclosed herein, in an amount effective to inhibit mammalian or non-mammalian gene expression, and a pharmaceutically acceptable carrier, and use thereof for treatment of any one of the diseases and disorders disclosed herein other than cancer. In some embodiments the mammalian gene is a human gene. In some embodiments the non-mammalian gene is involved in a mammalian disease, preferably a human disease.

Further provided are methods for treating or preventing the incidence or severity of any one of the diseases or conditions disclosed herein or for reducing the risk or severity of a disease or a condition disclosed herein in a subject in need thereof, wherein the disease or condition and/or a symptom or risk associated therewith is associated with expression of a mammalian or a non-mammalian gene the method comprising administering to a subject in need thereof a therapeutically effective amount of a compound disclosed herein. In a preferred embodiment the subject is a human subject. Provided herein are double-stranded nucleic acid molecules for therapy, wherein the therapy is other than cancer therapy.

### Oligonucleotide Synthesis

Using public and proprietary algorithms the sense and antisense sequences of potential double-stranded RNA molecules are generated.

The modified nucleic acid molecules are synthesized by any of the methods that are well known in the art for synthesis of ribonucleic (or deoxyribonucleic) oligonucleotides. Synthesis is commonly performed in a commercially available synthesizer (available, inter alia, from Applied Biosystems). Oligonucleotide synthesis is described for example in Beaucage and Iyer, Tetrahedron 1992; 48:2223-2311; Beaucage and Iyer, Tetrahedron 1993; 49: 6123-6194 and Caruthers, et. al., Methods Enzymol. 1987; 154: 287-313; the synthesis of thioates is, among others, described in Eckstein, Ann Rev. Biochem. 1985; 54: 367-402, the synthesis of RNA molecules is described in Sproat, in Humana Press 2005 edited by Herdewijn P.; Kap. 2: 17-31 and respective downstream processes are, among others, described in Pingoud et al., in IRL Press 1989 edited by Oliver R. W. A.; Kap. 7: 183-208.

Other synthetic procedures are known in the art, e.g. the procedures described in Usman et al., 1987, J. Am. Chem. Soc., 109, 7845; Scaringe et al., 1990, NAR., 18, 5433; Wincott et al., 1995, NAR. 23, 2677-2684; and Wincott et al., 1997, Methods Mol. Bio., 74, 59, may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

In some embodiments the oligonucleotides disclosed herein are synthesized separately and joined together post-synthetically, for example, by ligation (Moore et al., 1992, Science 256, 9923; Draper et al., International Patent Publication No. WO 93/23569; Shabarova et al., 1991, NAR 19, 4247; Bellon et al., 1997, Nucleosides & Nucleotides, 16, 951; Bellon et al., 1997, Bioconjugate Chem. 8, 204), or by hybridization following synthesis and/or de-protection.

Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the sphingolipid-polyalkylamine compounds disclosed herein, two or more such sequences can be synthesized and linked together for use.

In various embodiments some of the dsRNA molecules possess a terminal moiety covalently bound at the 5′-terminus of the antisense strand which is mismatched to the corresponding nucleotide in the target mRNA.

In one embodiment, provide are double-stranded nucleic acid (e.g. dsRNA, siRNA, siNA), which down-regulate the expression of mammalian or non-mammalian target genes. The double-stranded molecules comprise at least one TNA on the sense strand and or the antisense strand. In some embodiments the sense strand comprises a nucleotide sequence derived from the target RNA sequence, and the antisense strand is complementary to the sense strand. In general, some deviation from the target mRNA sequence is tolerated without compromising the siRNA activity (see e.g. Czauderna et al., 2003, NAR 31(11), 2705-2716). A dsRNA of the invention inhibits gene expression on a post-transcriptional level with or without destroying the mRNA. Without being bound by theory, dsRNA may target the mRNA for specific cleavage and degradation and/or may inhibit translation from the targeted message.

In one aspect, provided are nucleic acid molecules (e.g., siNA molecules) in which a) the nucleic acid molecule includes a sense strand and an antisense strand; b) each strand of the is independently 15 to 49 nucleotides in length; (c) a 15 to 49 nucleotide sequence of the antisense strand is complementary to a sequence of a target RNA; d) at least one sphingolipid-polyalkylamine conjugate is covalently attached at the 3′ terminus of the sense strand, at the 3′ terminus of the antisense strand or at the 5′ terminus of the sense strand; and e) 15 to 49 nucleotide sequence of the sense strand is complementary to the a sequence of the antisense strand and includes a 15 to 49 nucleotide sequence of a target RNA.

In some embodiments the antisense strand and the antisense strand are the same length. In some embodiments the antisense strand and the sense strand are 18-25 or 18-23 or 18-21 or 19-21 or 19 nucleotides in length.

### Pharmaceutical Compositions

While it is possible for the molecules disclosed herein to be administered as the raw chemical, it is preferable to present them as a pharmaceutical composition. Accordingly, provided herein is a pharmaceutical composition comprising one or more of the sphingolipid-polyalkylamine-oligonucleotide compounds disclosed herein; and a pharmaceutically acceptable carrier. In some embodiments the pharmaceutical composition comprises two or more modified compounds disclosed herein.

Further provided are pharmaceutical compositions comprising at least one compound, or salt of such compound, disclosed herein in an amount effective to inhibit a target gene expression; and a pharmaceutically acceptable carrier. The compound may be processed intracellularly by endogenous cellular complexes to produce one or more nucleic acid molecules disclosed herein.

Further provided are pharmaceutical compositions comprising a pharmaceutically acceptable carrier and one or more of the compounds disclosed herein in an amount effective to inhibit expression in a cell of a mammalian target gene.

In some embodiments, the sphingolipid-polyalkylamine oligonucleotide (e.g. sphingolipid-polyalkylamine dsRNA) compounds, or salts of such compounds, disclosed herein are the main active component in a pharmaceutical composition. In other embodiments a sphingolipid-polyalkylamine oligonucleotide (e.g. sphingolipid-polyalkylamine dsRNA) compound disclosed herein is one of the active components of a pharmaceutical composition containing two or more therapeutic agents, said pharmaceutical composition further being comprised of one or more sphingolipid-polyalkylamine oligonucleotide or dsRNA molecules which target one or more target genes or for example, a small molecule drug.

Further provided is a process of preparing a pharmaceutical composition, which comprises: providing one or more compound disclosed herein; and admixing said compound with a pharmaceutically acceptable carrier.

In a preferred embodiment, a sphingolipid-polyalkylamine oligonucleotide dsRNA compound disclosed herein used in the preparation of a pharmaceutical composition is admixed with a carrier in a pharmaceutically effective dose.

Also provided are kits, containers and formulations that include a sphingolipid-polyalkylamine dsRNA compound as provided herein for reducing expression of a target gene for administering or distributing the nucleic acid molecule to a patient. A kit may include at least one container and at least one label. Suitable containers include, for example, bottles, vials, syringes, and test tubes. The containers can be formed from a variety of materials such as glass, metal or plastic. In one embodiment, the container holds a sphingolipid-polyalkylamine dsRNA compounds as disclosed herein. Kits may further include associated indications and/or directions; reagents and other compositions or tools used for such purpose can also be included.

The container can alternatively hold a composition comprising an active agent that is effective for treating, diagnosis, prognosing or prophylaxing a condition and can have a sterile access port (for example the container can be an intravenous solution bag or a vial having a stopper pierceable by a hypodermic injection needle). The active agent in the composition can be a sphingolipid-polyalkylamine compound as disclosed herein.

A kit may further include a second container that includes a pharmaceutically-acceptable buffer and may further include other materials desirable from a commercial and user standpoint, including other buffers, diluents, filters, stirrers, needles, syringes, and/or package inserts with indications and/or instructions for use.

The container holding the sphingolipid-polyalkylamine dsRNA compound may include a package that is labeled, and the label may bear a notice in the form prescribed by a governmental agency, for example the Food and Drug Administration, which notice is reflective of approval by the agency under Federal law, of the manufacture, use, or sale of the polynucleotide material therein for human administration.

### Dosages

The useful dosage to be administered and the particular mode of administration will vary depending upon such factors as the cell type, or for in vivo use, the age, weight and the particular subject and region thereof to be treated, the particular nucleic acid and delivery method used, the therapeutic or diagnostic use contemplated, and the form of the formulation, for example, naked, suspension, emulsion, micelle or liposome, as will be readily apparent to those skilled in the art. Typically, dosage is administered at lower levels and increased until the desired effect is achieved.

A “therapeutically effective dose” for purposes herein is determined by considerations as are known in the art. The dose must be effective to achieve improvement including but not limited to improved survival rate or more rapid recovery, or improvement or alleviation of elimination of symptoms and other indicators as are selected as appropriate measures by those skilled in the art. The dsRNA disclosed herein can be administered in a single dose or in multiple doses.

A suitable dosage unit of nucleic acid molecules may be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day.

Suitable amounts of nucleic acid molecules may be introduced and these amounts can be empirically determined using standard methods. Effective concentrations of individual nucleic acid molecule species in the environment of a cell may be about 1 femtomolar, about 50 femtomolar, 100 femtomolar, 1 picomolar, 1.5 picomolar, 2.5 picomolar, 5 picomolar, 10 picomolar, 25 picomolar, 50 picomolar, 100 picomolar, 500 picomolar, 1 nanomolar, 2.5 nanomolar, 5 nanomolar, 10 nanomolar, 25 nanomolar, 50 nanomolar, 100 nanomolar, 500 nanomolar, 1 micromolar, 2.5 micromolar, 5 micromolar, 10 micromolar, 100 micromolar or more.

An appropriate dosage for a mammal may be from 0.01 ug to 1 g per kg of body weight (e.g., 0.1 ug, 0.25 ug, 0.5 ug, 0.75 ug, 1 ug, 2.5 ug, 5 ug, 10 ug, 25 ug, 50 ug, 100 ug, 250 ug, 500 ug, 1 mg, 2.5 mg, 5 mg, 10 mg, 25 mg, 50 mg, 100 mg, 250 mg, or 500 mg per kg).

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 0.1 mg to about 500 mg of an active ingredient. Dosage units may be adjusted for local delivery, for example for intravitreal delivery or for otic or transtympanic delivery.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy. For example, the amount of sphingolipid-polyalkylamine oligonucleotide compound to be delivered to each ear in order to treat auditory or vestibular disturbances and disorders is normally in the range of 5 micrograms to 5 mg total compound per ear, preferably 100 micrograms to 1 mg total compound per ear.

Pharmaceutical compositions that include the compounds disclosed herein may be administered once daily, qid, tid, bid, QD, or at any interval and for any duration that is medically appropriate. However, the therapeutic agent may also be dosed in dosage units containing two, three, four, five, six or more sub-doses administered at appropriate intervals throughout the day. In that case, the nucleic acid molecules contained in each sub-dose may be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. The dosage unit may contain a corresponding multiple of the daily dose. The composition can be compounded in such a way that the sum of the multiple units of nucleic acids together contains a sufficient dose.

### Delivery

The sphingolipid-polyalkylamine-oligonucleotide compounds disclosed herein are administered as the compound per se (i.e. as naked siRNA) or as pharmaceutically acceptable salt and are administered alone or as an active ingredient in combination with one or more pharmaceutically acceptable carrier, solvent, diluent, excipient, adjuvant and vehicle. In some embodiments, the sphingolipid-polyalkylamine-oligonucleotide compounds are delivered to the target tissue by direct application of the naked molecules prepared with a carrier or a diluent.

The term “naked compound” refers to sphingolipid-polyalkylamine-oligonucleotide compounds s that are free from any delivery vehicle that acts to assist, promote or facilitate entry into the cell, including viral sequences, viral particles, liposome formulations, lipofectin or precipitating agents and the like. For example, a sphingolipid-polyalkylamine-oligonucleotide compound in PBS is “naked”.

Pharmaceutically acceptable carriers, solvents, diluents, excipients, adjuvants and vehicles as well as implant carriers generally refer to inert, non-toxic solid or liquid fillers, diluents or encapsulating material not reacting with the sphingolipid-polyalkylamine-oligonucleotide compounds disclosed herein. The sphingolipid-polyalkylamine-oligonucleotide compounds disclosed herein may be delivered as a naked compound (oligonucleotide or oligonucleotide conjugated to lipophilic agent) or with a carrier or diluent or any delivery vehicle that acts to assist, promote or facilitate entry to the cell, enhance endosomal release and or increase tissue/cell retention. The carrier can coat oligonucleotides, be complexed with it or be delivered sequentially with the oligonucleotide provided the delivery is topical or local or, in case of systemic delivery, both oligonucleotide and carrier are targeted to the same type of cells. Carriers, or delivery vehicles refer to all those known in the art including but not limited to viral vectors, viral particles, liposome formulations, lipofectin or precipitating or complexing agents and the like. Delivery systems include but are not limited to surface-modified liposomes containing poly (ethylene glycol) lipids (PEG-modified, or long-circulating liposomes or stealth liposomes). The compounds may be formulated or complexed with biological and non-biological gels including collagen, poly-lactic acid (PLA) and derivatives thereof, poly-glycolic acid, poly(ε-caprolactone), poly(β-hydroxybutyrate), poly(β-hydroxyvalerate), polydioxanone, poly(ethylene terephthalate), poly(malic acid), poly(tartronic acid), poly(ortho esters), polyanhydrides, polycyanoacrylates, poly(phosphoesters), polyphhosphazenes, hyaluronidate, polysulfones, polyacrylamides, polymethacrylate, CarboPol and hydroxyapatite and combinations thereof and derivatives thereof. Other materials include polyethylenimine (e.g., linear or branched PEI) and/or polyethylenimine derivatives, including for example polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives, grafted PEIs such as galactose PEI, cholesterol PEI, antibody derivatized PEI, and polyethylene glycol PEI (PEG-PEI) derivatives thereof. Alternatively, the compounds may be delivered using a dendrimer, for example a peptide dendrimer or cationic dendrimer; or a nanoparticle including gold or magnetic nanoparticle or self assembling DNA nanoparticle and the like. The carriers may also comprise cell-targeting entities including but not limited to vitamins, cell surface receptor ligands, antibodies or aptamers, peptides and/or cell penetration peptides (CPP). Oligonucleotide/carrier formulations may be further presented as liquids, gels, creams, foams, aerosols and in certain embodiments contain additional penetration enhancers known in the art (e.g. skin penetration enhancers). Yin, et al., (2014, Nature Reviews Genetics 15:541-555) and Zhu and Mahato (2010, Expert Opin Drug Deliv. 7(10): 1209-1226) disclose various non-limiting examples of vehicles useful for delivery of oligonucleotide compounds.

Additionally, the compositions may include an artificial oxygen carrier, such as perfluorocarbon (PFCs) e.g. perfluorooctyl bromide (perflubron). Pharmaceutically acceptable ingredients include, without being limited to, one or more of buffering agent, preservative, surfactant, carrier, solvent, diluent, co-solvent, tonicity building/enhancing agent, viscosity building/enhancing agent, excipient, adjuvant and vehicle. In certain embodiments accepted preservatives such as benzalkonium chloride and disodium edetate (EDTA) are included in the compositions disclosed herein in concentrations sufficient for effective antimicrobial action.

In one specific embodiment, topical and transdermal formulations are preferred. In one specific embodiment formulations including hyaluronic acid are preferred, for example for application of the sphingolipid-polyalkylamine oligonucleotide compound to the ear.

Additional formulations for improved delivery of the compounds disclosed herein can include non-formulated compounds and compounds bound to targeting antibodies (Song et al., Antibody mediated in vivo delivery of small interfering RNAs via cell-surface receptors, Nat Biotechnol. 2005. 23(6):709-17) or aptamers. The naked compounds or the pharmaceutical compositions comprising the compounds disclosed herein are administered and dosed in accordance with good medical practice, taking into account the clinical condition of the individual patient, the disease to be treated, the site and method of administration, scheduling of administration, patient age, sex, body weight and other factors known to medical practitioners.

The sphingolipid-polyalkylamine-oligonucleotide compounds disclosed herein can be administered by any of the conventional routes of administration, for example orally, subcutaneously, topically or parenterally including intravenous, intraarterial, intramuscular, intraperitoneally. Other methods of administration include dermal, transtympanic and intranasal administration, intratracheal instillation and intratracheal inhalation, as well as infusion techniques. Implants of the compounds are also useful. Intraocular administration can be carried out, for example, by intravitreal injection, eye drops or implants. In certain embodiments, treatment of ocular disorders is accomplished by administering the conjugated oligonucleotide directly to the eye by ocular tissue injection such as periocular, conjunctival, subtenon, intracameral, intravitreal, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device present in for example, the cul-de-sac or implanted adjacent to the sclera (transscleral) or in the sclera (intrascleral) or within the eye. Intracameral injection may be through the cornea into the interior chamber to allow the agent to reach the trabecular meshwork. Intracanalicular injection may be into the venous collector channels draining Schlemm's canal or into Schlemm's canal.

Liquid forms are prepared for invasive administration, e.g. injection or for topical or local or non-invasive administration. The term injection includes subcutaneous, transdermal, intravenous, intramuscular, intrathecal, intraocular, transtympanic and other parental routes of administration. The liquid compositions include aqueous solutions, with and without organic co-solvents, aqueous or oil suspensions, emulsions with edible oils, as well as similar pharmaceutical vehicles. In a particular embodiment, the administration comprises intravitreal administration. In another embodiment, the administration comprises otic or transtympanic administration.

In some embodiments the sphingolipid-polyalkylamine-oligonucleotide compounds disclosed herein are formulated for non-invasive administration. In some embodiments the compounds disclosed herein are formulated as eardrops for topical administration to the ear. In some embodiments the dsRNA molecules disclosed herein are formulated as eye drops for topical administration to the surface of the eye. Further information on administration of the dsRNA molecules disclosed herein can be found in Tolentino et al., Retina 2004. 24:132-138; and Reich et al., Molecular Vision, 2003. 9:210-216. In addition, in certain embodiments the compositions disclosed herein are formed as aerosols, for example for intranasal administration. In certain embodiments the compositions disclosed herein are formed as nasal drops, for example for intranasal instillation. In some embodiments the compositions are formulated as ear drops.

The therapeutic compositions disclosed herein are preferably administered into the lung by inhalation of an aerosol containing these compositions/compounds, or by intranasal or intratracheal instillation of said compositions. For further information on pulmonary delivery of pharmaceutical compositions see Weiss et al., Human Gene Therapy 1999. 10:2287-2293; Densmore et al., Molecular therapy 1999. 1:180-188; Gautam et al., Molecular Therapy 2001. 3:551-556; and Shahiwala & Misra, AAPS PharmSciTech 2004. 24; 6(3):E482-6. Additionally, respiratory formulations for siRNA are described in U.S. Patent Application Publication No. 2004/0063654. Respiratory formulations for siRNA are described in US Patent Application Publication No. 2004/0063654.

In certain embodiments, oral compositions (such as tablets, suspensions, solutions) may be effective for local delivery to the oral cavity such as oral composition suitable for mouthwash for the treatment of oral mucositis.

In a particular embodiment, the sphingolipid-polyalkylamine-oligonucleotide compounds disclosed herein are formulated for intravenous administration for delivery to the kidney for the treatment of kidney disorders, e.g. acute renal failure (ARF), delayed graft function (DGF) and diabetic retinopathy. It is noted that the delivery of the compounds to the target cells in the kidney proximal tubules is particularly effective in the treatment of ARF and DGF.

Delivery of compounds into the brain is accomplished by several methods such as, inter alia, neurosurgical implants, blood-brain barrier disruption, lipid mediated transport, carrier mediated influx or efflux, plasma protein-mediated transport, receptor-mediated transcytosis, absorptive-mediated transcytosis, neuropeptide transport at the blood-brain barrier, and genetically engineering “Trojan horses” for drug targeting. The above methods are performed, for example, as described in “Brain Drug Targeting: the future of brain drug development”, W. M. Pardridge, Cambridge University Press, Cambridge, UK (2001).

In addition, in certain embodiments the compositions for use in the treatments disclosed herein are formed as aerosols, for example for intranasal administration.

Intranasal delivery for the treatment of CNS diseases has been attained with acetylcholinesterase inhibitors such as galantamine and various salts and derivatives of galantamine, for example as described in US Patent Application Publication No. 2006003989 and PCT Applications Publication Nos. WO 2004/002402 and WO 2005/102275. Intranasal delivery of nucleic acids for the treatment of CNS diseases, for example by intranasal instillation of nasal drops, has been described, for example, in PCT Application Publication No. WO 2007/107789.

### Methods of Treatment

In one aspect provided herein is a method of treating a subject suffering from a disorder associated with target gene expression comprising administering to the subject a therapeutically effective amount of a sphingolipid-polyalkylamine-oligonucleotide compound disclosed herein. In preferred embodiments the subject being treated is a warm-blooded animal and, in particular, mammal including human.

“Treating a subject” refers to administering to the subject a therapeutic substance effective to ameliorate symptoms associated with a disease, to lessen the severity or cure the disease, to slow down the progress of the disease, to prevent the disease from occurring or to postpone the onset of the disease. “Treatment” refers to both therapeutic treatment and prophylactic or preventative measures, wherein the object is to prevent a disorder, to delay the onset of the disorder or reduce the symptoms of a disorder. Those in need of treatment include those already experiencing the disease or condition, those prone to having the disease or condition, and those in which the disease or condition is to be prevented. The compounds disclosed herein are administered before, during or subsequent to the onset of the disease or condition.

A “therapeutically effective dose” refers to an amount of a pharmaceutical compound or composition which is effective to achieve an improvement in a subject or his physiological systems including, but not limited to, improved survival rate, more rapid recovery, improvement or elimination of symptoms, delayed onset of a disorder, slower progress of disease and other indicators as are selected as appropriate determining measures by those skilled in the art.

In some embodiments the sphingolipid-polyalkylamine is covalently attached to a siRNA. In some embodiments the sphingolipid-polyalkylamine is covalently attached to an antisense molecule. In some embodiments the sphingolipid-polyalkylamine is covalently attached to a shRNA. In some embodiments the sphingolipid-polyalkylamine is covalently attached to an aptamer. In some embodiments the sphingolipid-polyalkylamine is covalently attached to a synthetic mRNA.

In some embodiments the disease or condition is selected from hearing loss or a balance disease or disorder, an eye disease or disorder, a respiratory disease or disorder, a renal disease or disorder, fibrosis and an inflammatory disease or disorder.

In some embodiments, the compounds disclosed herein are useful in treating acute renal failure (ARF), Delayed Graft Function (DGF) after kidney transplantation, glaucoma, ocular ischemic conditions including anterior ischemic optic neuropathy, age-related macular degeneration (AMD), Ischemic Optic Neuropathy (ION), dry eye syndrome, acute respiratory distress syndrome (ARDS) and other acute lung and respiratory injuries, chronic obstructive pulmonary disease (COPD), primary graft failure, ischemia-reperfusion injury, reperfusion injury, reperfusion edema, allograft dysfunction, pulmonary reimplantation response and/or primary graft dysfunction (PGD) after organ transplantation, in particular in lung transplantation, organ transplantation including lung, liver, heart, pancreas, and kidney transplantation, nephro- and neurotoxicity, spinal cord injury, brain injury, neurodegenerative disease or condition, pressure sores, oral mucositis fibrotic conditions including liver fibrosis, lung fibrosis; ocular neuropathy, elevated intraocular pressure (TOP), Sjögrens Syndrome, diabetic retinopathy (DR), diabetic macular edema (DME), optic neuritis, central retinal vein occlusion, brunch retinal vein occlusion, optic nerve injury, retinopathy of prematurity (ROP), retinitis pigmentosa (RP), retinal ganglion degeneration, macular degeneration, hereditary optic neuropathy, Leber's hereditary optic neuropathy, neuropathy due to a toxic agent and neuropathy caused by an adverse drug reaction or a vitamin deficiency; and Meniere's disease. Such methods involve administering to a mammal in need of such treatment a prophylactically or therapeutically effective amount of one or more sphingolipid-polyalkylamine-oligonucleotide compounds, which modulate expression or activity of at least one such gene.

Fibrotic diseases are generally characterized by the excess deposition of a fibrous material within the extracellular matrix, which contributes to abnormal changes in tissue architecture and interferes with normal organ function.

All tissues damaged by trauma respond by the initiation of a wound-healing program. Fibrosis, a type of disorder characterized by excessive scarring, occurs when the normal self-limiting process of wound healing response is disturbed, and causes excessive production and deposition of collagen. As a result, normal organ tissue is replaced with scar tissue, which eventually leads to the functional failure of the organ. Fibrosis may be initiated by diverse causes and in various organs. Liver cirrhosis, pulmonary fibrosis, sarcoidosis, keloids and kidney fibrosis are all chronic conditions associated with progressive fibrosis, thereby causing a continuous loss of normal tissue function. Acute fibrosis (usually with a sudden and severe onset and of short duration) occurs as a common response to various forms of trauma including accidental injuries (particularly injuries to the spine and central nervous system), infections, surgery, ischemic illness (e.g. cardiac scarring following heart attack), burns, environmental pollutants, alcohol and other types of toxins, acute respiratory distress syndrome, radiation and chemotherapy treatments).

Fibrosis, a fibrosis related pathology or a pathology related to aberrant crosslinking of cellular proteins may all be treated by the compounds disclosed herein. Fibrotic diseases or diseases in which fibrosis is evident (fibrosis related pathology) include both acute and chronic forms of fibrosis of organs, including all etiological variants of the following: pulmonary fibrosis, including interstitial lung disease and fibrotic lung disease, liver fibrosis, cardiac fibrosis including myocardial fibrosis, kidney fibrosis including chronic renal failure, skin fibrosis including scleroderma, keloids and hypertrophic scars; myelofibrosis (bone marrow fibrosis); all types of ocular scarring including proliferative vitreoretinopathy (PVR) and scarring resulting from surgery to treat cataract or glaucoma; inflammatory bowel disease of variable etiology, macular degeneration, Grave's ophthalmopathy, drug induced ergotism, keloid scars, scleroderma, psoriasis, and collagenous colitis.

The diseases and disorders relevant to the present disclosure may be classified in more than one group.

Inflammatory disease as used herein includes Ulcerative colitis, Crohn's disease, rheumatoid arthritis and multiple sclerosis. Inflammatory bowel disease (IBD) refers to two chronic syndromes: ulcerative colitis and Crohn's disease. IBD presents with any of the following symptoms: abdominal pain, vomiting, diarrhea, rectal bleeding, severe internal cramps/muscle spasms in the region of the pelvis and weight loss. Anemia is the most prevalent complication. Associated complaints or diseases include arthritis, pyoderma gangrenosum, and primary sclerosing cholangitis. Diagnosis is generally by assessment of inflammatory markers in stool followed by colonoscopy with biopsy of pathological lesions.

Multiple sclerosis (MS) is an inflammatory disease in which myelin sheaths around axons of the brain and spinal cord are damaged, leading to loss of myelin and scarring.

Rheumatoid arthritis (RA) is an autoimmune disease that results in a chronic, systemic inflammatory disorder that may affect many tissues and organs, but principally attacks flexible (synovial) joints.

One with skill in the art will be able to identify relevant inflammatory disease target genes and generate an active antisense or dsRNA molecule to target the gene or gene transcription product.

“Respiratory disorders” refers to conditions, diseases or syndromes of the respiratory system including but not limited to pulmonary disorders of all types including chronic obstructive pulmonary disease (COPD), emphysema, chronic bronchitis, and asthma inter alia. Emphysema and chronic bronchitis may occur as part of COPD or independently. In various embodiments provided are methods and compositions useful in preventing or treating primary graft failure, ischemia-reperfusion injury, reperfusion injury, reperfusion edema, allograft dysfunction, pulmonary reimplantation response and/or primary graft dysfunction (PGD) after organ transplantation, in particular in lung transplantation, in a subject in need thereof

One with skill in the art will be able to identify relevant respiratory disease target genes and generate an active antisense or dsRNA molecule to target the gene or gene transcription product.

“Microvascular disorder” refers to any condition that affects microscopic capillaries and lymphatics, in particular vasospastic diseases, vasculitic diseases and lymphatic occlusive diseases. Examples of microvascular disorders include, inter alia: eye disorders such as Amaurosis Fugax (embolic or secondary to SLE), apla syndrome, Prot CS and ATIII deficiency, microvascular pathologies caused by IV drug use, dysproteinemia, temporal arteritis, ischemic optic neuropathy (ION), non-arteritic ischemic optic neuropathy (NAION), anterior ischemic optic neuropathy (AION), optic neuritis (primary or secondary to autoimmune diseases), glaucoma, von Hippel Lindau syndrome, corneal disease, corneal transplant rejection cataracts, Eales' disease, frosted branch angiitis, encircling buckling operation, uveitis including pars planitis, choroidal melanoma, choroidal hemangioma, optic nerve aplasia; retinal conditions such as retinal artery occlusion, retinal vein occlusion, retinopathy of prematurity, HIV retinopathy, Purtscher retinopathy, retinopathy of systemic vasculitis and autoimmune diseases, diabetic retinopathy, hypertensive retinopathy, radiation retinopathy, branch retinal artery or vein occlusion, idiopathic retinal vasculitis, aneurysms, neuroretinitis, retinal embolization, acute retinal necrosis, Birdshot retinochoroidopathy, long-standing retinal detachment; systemic conditions such as Diabetes mellitus, diabetic retinopathy (DR), diabetes-related microvascular pathologies (as detailed herein), hyperviscosity syndromes, aortic arch syndromes and ocular ischemic syndromes, carotid-cavernous fistula, multiple sclerosis, systemic lupus erythematosus, arteriolitis with SS-A autoantibody, acute multifocal hemorrhagic vasculitis, vasculitis resulting from infection, vasculitis resulting from Behçet's disease, sarcoidosis, coagulopathies, neuropathies, nephropathies, microvascular diseases of the kidney, and ischemic microvascular conditions, inter alia.

Microvascular disorders may comprise a neovascular element. The term “neovascular disorder” refers to those conditions where the formation of blood vessels (neovascularization) is harmful to the patient. Examples of ocular neovascularization include: retinal diseases (diabetic retinopathy, diabetic Macular Edema, chronic glaucoma, retinal detachment, and sickle cell retinopathy); rubeosis iritis; proliferative vitreo-retinopathy; inflammatory diseases; chronic uveitis; neoplasms (retinoblastoma, pseudoglioma and melanoma); Fuchs' heterochromic iridocyclitis; neovascular glaucoma; corneal neovascularization (inflammatory, transplantation and developmental hypoplasia of the iris); neovascularization following a combined vitrectomy and lensectomy; vascular diseases (retinal ischemia, choroidal vascular insufficiency, choroidal thrombosis and carotid artery ischemia); neovascularization of the optic nerve; and neovascularization due to penetration of the eye or contusive ocular injury. In various embodiments all these neovascular conditions are treated using the compounds and pharmaceutical compositions disclosed herein.

One with skill in the art will be able to identify relevant microvascular disease target genes and generate an active antisense or dsRNA molecule to target the gene or gene transcription product.

“Eye disease” refers to conditions, diseases or syndromes of the eye including but not limited to any conditions involving choroidal neovascularization (CNV), wet and dry AMD, ocular histoplasmosis syndrome, angiod streaks, ruptures in Bruchs membrane, myopic degeneration, ocular tumors, retinal degenerative diseases and retinal vein occlusion (RVO). In various embodiments, conditions disclosed herein, such as DR, which are regarded as either a microvascular disorder or an eye disease, or both, under the definitions presented herein, are treated according to the methods disclosed herein.

One with skill in the art will be able to identify relevant eye disease target genes and generate an active antisense or dsRNA molecule to target the gene or gene transcription product.

An ear disease or disorder includes ear disorder, including, inter alia, balance disorders and hearing loss arising from chemical-induced ototoxicity, acoustic trauma and presbycusis; and microbial infections. International publication WO2013020097 to the assignee of the present application and incorporated by reference in its entirety provides siRNA molecules useful in generating sphingolipid-polyalkylamine oligonucleotide compounds useful for treating ear diseases and disorders.

One with skill in the art will be able to identify relevant ear disease target genes and generate an active antisense or dsRNA molecule to target the gene or gene transcription product.

Diseases and disorders of the nervous system include, inter alia, spinal cord injury and peripheral nerve injury. US patent publication US20120252875 to the assignee of the present application and incorporated by reference in its entirety provides siRNA molecules useful in generating sphingolipid-polyalkylamine oligonucleotide compounds useful for treating diseases and disorders of the nervous system.

One with skill in the art will be able to identify relevant nervous system disease target genes and generate an active antisense or dsRNA molecule to target the gene or gene transcription product.

Additionally, provided is a method of down-regulating the expression of a target gene by at least 20%, 30%, 40%, or 50%, preferably 60%, 70% or more as compared to a control comprising contacting target mRNA with one or more of the sphingolipid-polyalkylamine compounds disclosed herein. In various embodiments the sphingolipid-polyalkylamine compounds down-regulate target gene expression whereby the down-regulation is selected from the group comprising down-regulation of gene function, down-regulation of polypeptide expression and down-regulation of mRNA expression. Down regulation is examined by, for example, an enzymatic assay or a binding assay with a known interactor of the native gene/polypeptide, inter alia), inhibition of target protein (which is examined by, for example, Western blotting, ELISA or immuno-precipitation, inter alia) and inhibition of target mRNA expression (which is examined by, for example, Northern blotting, quantitative RT-PCR, in-situ hybridization or microarray hybridization, inter alia).

In additional embodiments provided is a method of treating a subject suffering from or susceptible to any disease or disorder accompanied by an elevated level of a mammalian or non-mammalian target gene, the method comprising administering to the subject a sphingolipid-polyalkylamine dsRNA compound disclosed herein in a therapeutically effective dose thereby treating the subject.

Provided herein are a sphingolipid-polyalkylamine dsRNA compounds for use in therapy, in particular for use where down-regulation of expression of a mammalian or non-mammalian target gene is beneficial.

By “exposure to a toxic agent” is meant that the toxic agent is made available to, or comes into contact with, a mammal. A toxic agent can be toxic to the nervous system. Exposure to a toxic agent can occur by direct administration, e.g., by ingestion or administration of a food, medicinal, or therapeutic agent, e.g., a chemotherapeutic agent, by accidental contamination, or by environmental exposure, e g., aerial or aqueous exposure.

In other embodiments the sphingolipid-polyalkylamine dsRNA compounds and methods disclosed herein are useful for treating or preventing the incidence or severity of other diseases and conditions in a subject.

Without limitation a mammalian target gene is selected from the group consisting of p53 (TP53), TP53BP2, LRDD, CYBA, ATF3, CASP2 (Caspase 2), NOX3, HRK; C1QBP, BNIP3, MAPK8; Rac1, GSK3B, CD38, STEAP4, BMP2a; GJA1, TYROBP, CTGF, SPP1, RTN4R, ANXA2, RHOA, DUOX1, SLC5A1, SLC2A2, AKR1B1, SORD, SLC2A1, MME, NRF2, SRM, REDD2 (RTP801L), REDD1 (RTP801), NOX4, MYC, PLK1, ESPL1, HTRA2, KEAP1, p66, ZNHIT1, LGALS3, CYBB (NOX2), NOX1, NOXO1, ADRB1, HI 95, ARF1, ASPP1, SOX9, FAS, FASLG, Human MLL, AF9, CTSD, CAPNS1, CD80, CD86, HES1, HES5, HEY1, HEY2, CDKN1B (p27), ID1, ID2, ID3, CDKN2A (p16), NOTCH, PSEN, Caspase 1, Caspase 3, Caspase 4, Caspase 5, Caspase 6, Caspase 7, Caspase 8, Caspase 9, Caspase 10, Caspase 12, Caspase 14, Apaf-1, Nod1, Nod2, Ipaf, DEFCAP, RAIDD, RICK, Bcl10, ASC, TUCAN, ARC, CLARP, FADD, DEDD, DEDD2, Cryopirin, PYC1, Pyrin, TRADD, UNC5a, UNC5b, UNC5c, ZUD, p84N5, LRDD, CDK1, CDK2, CDK4, CDK5, CDK9, PITSLRE A, CHK2, LATS1, Prk, MAP4K1, MAP4K2, STK4, SLK, GSK3alpha, GSK3beta, MEKK1, MAP3K5 (Ask1), MAP3K7, MAP3K8, MAP3K9, MAP3K10, MAP3K11, MAP3K12, DRP-1, MKK6, p38, JNK3, DAPK1, DRAK1, DRAK2, IRAK, RIP, RIP3, RIP5, PKR, IRE1, MSK1, PKCalpha, PKCbeta, PKCdelta, PKCepsilon, PKCeta, PKCmu, PKCtheta, PKCzeta, CAMK2A, HIPK2, LKB1, BTK, c-Src, FYN, Lck, ABL2, ZAP70, TrkA, TrkC, MYLK, FGFR2, EphA2, AATYK, c-Met, RET, PRKAA2, PLA2G2A, SMPD1, SMPD2, SPP1, FAN, PLCG2, IP6K2, PTEN, SHIP, AIF, AMID, Cytochrome c, Smac, HtrA2, TSAP6, DAP-1, FEM-, DAP-3, Granzyme B, DIO-1, DAXX, CAD, CIDE-A, CIDE-B, Fsp27, Ape1, ERCC2, ERCC3, BAP31, Bit1, AES, Huntingtin, HIP1, hSir2, PHAP1, GADD45b, GADD34, RAD21, MSH6, ADAR, MBD4, WW45, ATM, mTOR, TIP49, diubiquitin/FAT10, FAF1, p193, Scythe/BAT3, Amida, IGFBP-3, TDAG51, MCG10, PACT, p52/RAP, ALG2, ALG3, presenelin-1, PSAP, AIP1/Alix, ES18, mda-7, p14ARF, ANTI, p33ING1, p33ING2, p53AIP1, p53DINP1, MGC35083, NRAGE, GRIM19, lipocalin 2, glycodelin A, NADE, Porimin, STAG1, DAB2, Galectin-7, Galectin-9, SPRC, FLJ21908, WWOX, XK, DKK-1, Fzd1, Fzd2, SARP2, axin 1, RGS3, DVL1, NFkB2, IkBalpha, NF-ATC1, NF-ATC2, NF-ATC4, zf3/ZNF319, Egr1, Egr2, Egr3, Sp1, TIEG, WT1, Zac1, Icaros, ZNF148, ZK1/ZNF443, ZNF274, WIG1, HIVEP1, HIVEP3, Fliz1, ZPR9, GATA3, TR3, PPARG, CSMF, RXRa, RARa, RARb, RARg, T3Ra, Erbeta, VDR, GR/GCCR, p53, p73alpha, p63 (human [ta alpha, ta beta, ta gamma, da alpha, a beta, da gamma], 53BP2, ASPP1, E2F1, E2F2, E2F3, alpha, TCF4, c-Myc, Max, Mad, MITF, Id2, Id3, Id4, c-Jun, c-Fos, ATF3, NF-IL6, CHOP, NRF1, c-Maf, Bach2, Msx2, Csx, HoxaS, Ets-1, PU1/Spi1, Ets-2, ELK1, TEL1, c-Myb, TBXS, IRF1, IRF3, IRF4, IRF9, AP-2 lpha, FKHR, FOXO1A, FKHRL1, FOXO3a, AFX1, MLLT7, Tip60, BTG1, AUF1, HNRPD, TIA1, NDG1, PCBP4, MCG10, FXR2, TNFR2, LTbR, CD40, CD27, CD30, 4-1BB, TNFRSF19, XEDAR, Fn14, OPG, DcR3, FAS, TNFR1, WSL-1, p75NTR, DR4, DR5, DR6, EDAR, TNF lpha, FAS ligand, TRAIL, Lymphotoxin alpha, Lymphotoxin beta, 4-1BBL, RANKL, TL1, TWEAK, LIGHT, APRIL, IL-1-alpha, IL-1-beta, IL-18, FGF8, IL-2, IL-21, IL-5, IL-4, IL-6, LIF, IL-12, IL-7, IL-10, IL-19, IL-24, IFN alpha, IFN beta, IFN gamma, M-CSF, Prolactinm, TLR2, TLR3, TLR4, MyD88, TRIF, RIG-1, CD14, TCR alpha, CD3 gamma, CD8, CD4, CD7, CD19, CD28, CTLA4, SEMA3A, SEMA3B, HLA-A, HLA-B, HLA-L, HLA-Dmalpha, CD22, CD33, CALL, DCC, ICAM1, ICAM3, CD66a, PVR, CD47, CD2, Thy-1, SIRPa1, CD5, E-cadherin, ITGAM, ITGAV, CD18, ITGB3, CD9, IgE Fc R beta, CD82, CD81, PERP, CD24, CD69, KLRD1, galectin 1, B4GALT1, C1q alpha, C5R1, MIP1alpha, MIP1beta, RANTES, SDF1, XCL1, CCCKR5, OIAS/OAS1, INDO, MxA, IFI16, AIM2, iNOS, HB-EGF, HGF, MIF, TRAF3, TRAF4, TRAF6, PAR-4, IKKGamma, FIP2, TXBP151, FLASH, TRF1, IEX-1S, Dok1, BLNK, CIN85, Bif-1, HEF1, Vav1, RasGRP1, POSH, Rac1, RhoA, RhoB, RhoC, ALG4, SPP1, TRIP, SIVA, TRABID, TSC-22, BRCA1, BARD1, 53BP1, MDC1, Mdm4, Siah-1, Siah-2, RoRet, TRIM35, PML, RFWD1, DIP1, Socs1, PARC, USP7, CYLD, TTR, SERPINH1 (HSP47) or any combination. Other useful target genes are genes of plant origin, fungal origin and microbial origin including viral, bacterial, and mycoplasmal genes.

Ear Disorders: in some embodiments, the sphingolipid-polyalkylamine dsRNA compounds are useful in treating a patient suffering from or at risk of various otic disorders, including auditory and vestibular disorders and diseases. Ear disorders include hearing loss induced for example by ototoxins, excessive noise or ageing. Middle and inner ear disorders produce many of the same symptoms, and a disorder of the middle ear may affect the inner ear and vice versa.

In addition to hearing loss, ear disorders include myringitis, an eardrum infection caused by a variety of viruses and bacteria; temporal bone fracture for example due to a blow to the head; auditory nerve tumors (acoustic neuroma, acoustic neurinoma, vestibular schwannoma, eighth nerve tumor). In various embodiments, the methods and compositions disclosed herein are useful in treating various conditions of hearing loss. Without being bound by theory, the hearing loss may be due to apoptotic inner ear hair cell damage or loss (Zhang et al., Neuroscience 2003. 120:191-205; Wang et al., J. Neuroscience 23((24):8596-8607), wherein the damage or loss is caused by infection, mechanical injury, loud sound (noise), aging (presbycusis), or chemical-induced ototoxicity.

By “ototoxin” in the context disclosed herein is meant a substance that through its chemical action injures, impairs or inhibits the activity of the sound receptors component of the nervous system related to hearing, which in turn impairs hearing (and/or balance). In the context of the present invention, ototoxicity includes a deleterious effect on the inner ear hair cells. Ototoxins include therapeutic drugs including antineoplastic agents, salicylates, loop-diuretics, quinines, and aminoglycoside antibiotics, contaminants in foods or medicinals, and environmental or industrial pollutants. Typically, treatment is performed to prevent or reduce ototoxicity, especially resulting from or expected to result from administration of therapeutic drugs. Preferably a compounds or composition disclosed herein is given immediately after the exposure to prevent or reduce the ototoxic effect. More preferably, treatment is provided prophylactically, either by administration of the pharmaceutical composition of the invention prior to or concomitantly with the ototoxic pharmaceutical or the exposure to the ototoxin. Incorporated herein by reference are chapters 196, 197, 198 and 199 of The Merck Manual of Diagnosis and Therapy, 14th Edition, (1982), Merck Sharp & Dome Research Laboratories, N.J. and corresponding chapters in the most recent 16th edition, including Chapters 207 and 210) relating to description and diagnosis of hearing and balance impairments.

Ototoxicity is a dose-limiting side effect of antibiotic administration. Ototoxic aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include neomycin B, kanamycin A, kanamycin B, gentamicin C1, gentamicin C1a, and gentamicin C2, and the like that are known to have serious toxicity, particularly ototoxicity and nephrotoxicity, which reduce the usefulness of such antimicrobial agents (see Goodman and Gilman's The Pharmacological Basis of Therapeutics, 6th ed., A. Goodman Gilman et al., eds; Macmillan Publishing Co., Inc., New York, pp. 1169-71 (1980)).

Ototoxicity is also a serious dose-limiting side-effect for anti-cancer agents. Ototoxic neoplastic agents include but are not limited to vincristine, vinblastine, cisplatin and cisplatin-like compounds and taxol and taxol-like compounds. Cisplatin-like compounds include carboplatin (Paraplatin®), tetraplatin, oxaliplatin, aroplatin and transplatin inter alia and are platinum based chemotherapeutics.

Diuretics with known ototoxic side-effect, particularly “loop” diuretics include, without being limited to, furosemide, ethacrylic acid, and mercurials. Ototoxic quinines include but are not limited to synthetic substitutes of quinine that are typically used in the treatment of malaria. In some embodiments the hearing disorder is side-effect of inhibitors of type 5 phosphodiesterase (PDE-5), including sildenafil (Viagra®), vardenafil (Levitra®) and tadalafil (Cialis).

Salicylates, such as aspirin, are the most commonly used therapeutic drugs for their anti-inflammatory, analgesic, anti-pyretic and anti-thrombotic effects. Unfortunately, they too have ototoxic side effects. They often lead to tinnitus (“ringing in the ears”) and temporary hearing loss. Moreover, if the drug is used at high doses for a prolonged time, the hearing impairment can become persistent and irreversible.

In some embodiments a method is provided for treatment of infection of a mammal by administration of an aminoglycoside antibiotic, the improvement comprising administering a therapeutically effective amount of one or more compound disclosed herein which down-regulate expression a target gene, to the subject in need of such treatment to reduce or prevent ototoxin-induced hearing impairment associated with the antibiotic.

The compounds and composition described herein are also effective in the treatment of acoustic trauma or mechanical trauma, preferably acoustic or mechanical trauma that leads to inner ear hair cell loss. With more severe exposure, injury can proceed from a loss of adjacent supporting cells to complete disruption of the organ of Corti. Death of the sensory cell can lead to progressive Wallerian degeneration and loss of primary auditory nerve fibers. The methods provided are useful in treating acoustic trauma caused by a single exposure to an extremely loud sound, or following long-term exposure to everyday loud sounds above 85 decibels, for treating mechanical inner ear trauma, for example, resulting from the insertion of an electronic device into the inner ear or for preventing or minimizing the damage to inner ear hair cells associated with the operation.

Another type of hearing loss is presbycusis, which is hearing loss that gradually occurs in most individuals as they age. About 30-35 percent of adults between the ages of 65 and 75 years and 40-50 percent of people 75 and older experience hearing loss. The compounds and compositions disclosed herein are useful in preventing, reducing or treating the incidence and/or severity of inner ear disorders and hearing impairments associated with presbycusis.

Provided is a method of treating a subject suffering from or at risk of an ear disorder which comprises topically administering to the canal of the subject's ear a compound or pharmaceutical composition comprising a compound described herein, thereby reducing expression of a gene associated with the disorder in the ear of the subject in an amount effective to treat the subject. Further provided is a method of treating a subject suffering from or at risk of an ear disorder which comprises transtympanically administering to the canal of the subject's ear a compound or pharmaceutical composition described herein, thereby reducing expression of a gene associated with the disorder in the ear of the subject in an amount effective to treat the subject. In one embodiment, the compound is delivered via a posterior semicircular canalostomy. In one embodiment, the compound is delivered as ear drops.

Diseases and Disorders of the Vestibular System: In various embodiments the nucleic acid compounds and pharmaceutical compositions disclosed herein are useful for treating disorders and diseases affecting the vestibular system in which expression of HES1, HES5, HEY1, HEY2, ID1, ID2, ID3, CDKN1B, CDKN2A, GSK3B or NOTCH1 is detrimental, for example Meniere's Disease. The vestibular sensory system in most mammals, including humans, contributes to balance, and to a sense of spatial orientation and stability. Together with the cochlea it constitutes the labyrinth of the inner ear. The vestibular system comprises two components: the semicircular canal system, which indicate rotational movements; and the otoliths, which indicate linear accelerations.

Further provided is a method of a method of treating a vestibular disorder in a subject, comprising administering to the subject at least one sphingolipid-polyalkylamine oligonucleotide compound which functions as an activator of atonal gene (Atohl), thereby treating the vestibular disorder in the subject. Further provided is a method of promoting regeneration of sensory hair cells in the vestibulum of the inner ear of a subject, comprising administering to the subject a therapeutically effective amount at least one sphingolipid-polyalkylamine oligonucleotide activator of Atohl, thereby promoting regeneration of sensory hair cells in the vestibulum of the subject. Meniere's Disease: Meniere's disease, also known as idiopathic endolymphatic hydrops (ELH), is a disorder of the inner ear resulting in vertigo and tinnitus, and eventual neuronal damage leading to hearing loss. Meniere's disease may affect one or both of a subject's ears. The primary morbidity associated with Meniere's disease is the debilitating nature of vertigo and the progressive hearing loss. Current therapies have not been successful at preventing progression of neuronal degeneration and associated hearing loss. A therapeutic treatment, which would protect the neurons of the inner ear including the vestibulocochlear nerve from damage and/or induce regeneration of the vestibulocochlear nerve and thereby attenuate or prevent hearing loss in Meniere's patients would be highly desirable. The compounds, compositions, methods and kits provided herein are useful in treating subjects at risk of or suffering from Meniere's disease.

In various embodiments the compounds and pharmaceutical compositions of the invention are useful in treating or preventing various diseases, disorders and injury that affect the ear, such as, without being limited to, the diseases, disorders and injury that are disclosed herein below. Without being bound by theory, it is believed that the molecules of the present invention prevent death or various types of cells within the ear.

### Combination Therapy

The methods of treatment disclosed herein include administering an oligonucleotide compound (i.e. sphingolipid-polyalkylamine oligonucleotide) disclosed herein alone or in combination with one or more additional compounds, such as a substance which improves the pharmacological properties of the oligonucleotide compound, or a therapeutically active agent known to be effective in the treatment of a subject suffering from or susceptible to any of the hereinabove mentioned diseases and disorders.

Therefore, provided are pharmaceutical compositions comprising an oligonucleotide compound (i.e. sphingolipid-polyalkylamine oligonucleotide) disclosed herein in combination with at least one additional therapeutically active agent.

By “in conjunction with” or “in combination with” is meant that the oligonucleotide compound is administered simultaneously or sequentially (either prior to or subsequent to) with administration of the additional therapeutically active agent. Accordingly, the individual components of such a combination are administered either simultaneously or sequentially from the same or separate pharmaceutical formulations.

Accordingly, in another embodiment, an additional therapeutically active agent is administered in conjunction with the oligonucleotide compound disclosed herein. In addition, the oligonucleotide compounds disclosed herein are used in the preparation of a medicament for use as adjunctive therapy with a second therapeutically active compound, and optionally a third or fourth therapeutically active compound to treat such conditions. Appropriate doses of known second therapeutically active agents, optionally third or fourth therapeutically active agent, for use in combination with the oligonucleotide compound disclosed herein are readily appreciated by those skilled in the art. For example, the second and optionally third or fourth therapeutically active agent may be an oligonucleotide (e.g. dsNA, ssNA, aptamer and the like), an oligonucleotide conjugated to a lipophilic agent (vitamin E, cholesterol, sphingolipid), an antibody or fragment thereof, a small molecule, a peptide or derivative thereof and the like.

In some embodiments the combinations referred to above are presented for use in the form of a single pharmaceutical formulation. In other embodiments, the combinations referred to above are presented for use in the form of multiple pharmaceutical formulations.

As is the case for the sphingolipid-polyalkylamine oligonucleotide compounds, which are preferably administered locally to for example, the ear, the eye, the lung etc. a second therapeutically active agent can be administered by the same route or any other suitable route, for example, by transtympanic, intravitreal, oral, buccal, inhalation, sublingual, rectal, vaginal, transurethral, nasal, otic, ocular, topical, percutaneous (i.e., transdermal), or parenteral (including intravenous, intramuscular, subcutaneous, and intracoronary) administration.

In some embodiments, a sphingolipid-polyalkylamine oligonucleotide compound disclosed herein and a second therapeutically active agent (dsNA or other) are administered by the same route, either provided in a single composition as two or more different pharmaceutical compositions. However, in other embodiments, a different route of administration for the sphingolipid-polyalkylamine oligonucleotide compound disclosed herein and the second therapeutically active agent is either possible or preferred. Persons skilled in the art are aware of the best modes of administration for each therapeutic agent, either alone or in combination.

The treatment regimen according to the disclosure herein is carried out, in terms of administration mode, timing of the administration, and dosage, so that the functional recovery of the subject from the adverse consequences of the conditions disclosed herein is improved or so as to postpone the onset of a disorder. The amount of active ingredient that can be combined with a carrier to produce a single dosage form varies depending upon the host treated and the particular mode of administration.

Dosage unit forms for monotherapy or combination therapy generally contain between from about 0.001 mg (1 μg) to about 50 mg of an active ingredient. Dosage units may be adjusted for local delivery, for example for transtympanic delivery. For treatment of auditory and vestibular function diseases and disorders, the amount to be delivered to each ear is normally in the range of 5 micrograms to 5 mg total compound per ear, preferably 100 micrograms to 1 mg total compound per ear. For treatment of ocular diseases and disorders, the amount to be delivered to an eye is normally in the range of 0.001-50 mg/dose total compound per eye, preferably 0.01 to about 5 mg total compound per eye. If adminstered as eye drops, the dose is from about 1 ug (microgram) to 1 mg per drop, or from about 50-500 ug/drop. For intravitreal injection, a dose is about 0.01 mg to about 50 mg/injection or about 0.1 mg to about 5 mg compound per injection.

The invention has been described in an illustrative manner, and it is to be understood that the terminology used is intended to be in the nature of words of description rather than of limitation.

Modifications and variations of the present invention are possible in light of the above teachings. It is, therefore, to be understood that within the scope of the appended claims, the invention can be practiced otherwise than as specifically described.

The present invention is illustrated in detail below with reference to examples, but is not to be construed as being limited thereto.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ (in cell) PCR in combination with Flow Cytometry is useful for detection of cells containing specific DNA and mRNA sequences (Testoni et al., Blood 1996, 87:3822.) Methods of performing RT-PCR and qPCR are also well known in the art.

### Example 1

### Selection and Generation of dsNA Sense Strand and Antisense Strand Sequences

Using proprietary algorithms and the known sequence of a target RNA, single stranded and double stranded oligonucleotides were generated. In some embodiments, 18-mer and 19-mer sequences are selected for generating dsNA molecules. For dsNA compounds, the antisense strand sequences generated using this method are fully or substantially complementary to a section of target RNA sequence. In some embodiments the antisense sequence is fully complementary to a section of the corresponding RNA sequence. For generating some of the exemplary sphingolipid-polyalkylamine oligonucleotide compounds disclosed herein, the nucleotide at the 5′ terminal position (5′ terminus) of the antisense strand (N)x (position 1) is substituted to generate a double-stranded nucleic acid molecule of with a mismatch to the target RNA. In some embodiments, the nucleotide at the 3′ terminal position (3′ terminus) of the sense strand (N′)y is substituted to be fully complementary with the antisense strand.

In general, the double-stranded nucleic acid molecules having specific sequences that are selected for in vitro testing are specific for human and a second species such as rat, mouse non-human primate or rabbit genes.

The exemplary compounds disclosed herein were selected and designed to target Rac1 (Homo sapiens ras-related C3 botulinum toxin substrate 1 (rho family, small GTP binding protein Rac1) (RAC1), transcript variant Rac1, mRNA) gi|156071503|ref|NM_006908.4| SEQ ID NO:1; HES5 (Homo sapiens hairy and enhancer of split 5 (Drosophila) gi|145301612|ref|NM_001010926.3| SEQ ID NO:2; HEY2 (hairy/enhancer-of-split related with YRPW motif 2; gi|105990529|ref|NM_012259.2| SEQ ID NO:3, MYD88 (Homo sapiens myeloid differentiation primary response gene (88) (MYD88)) including all splice variants (gi|197276653|ref|NM_002468.4| SEQ ID NO:4 Homo sapiens myeloid differentiation primary response 88 (MYD88), transcript variant 2, mRNA; gi|28954652|ref|NM_001172569.1| SEQ ID NO:5 Homo sapiens myeloid differentiation primary response 88 (MYD88), transcript variant 4, mRNA; gi|289546502|ref|NM_001172567.1| SEQ ID NO:6 Homo sapiens myeloid differentiation primary response 88 (MYD88), transcript variant 1, mRNA; gi|289546499|ref|NM_001172566.1 SEQ ID NO:7 Homo sapiens myeloid differentiation primary response 88 (MYD88), transcript variant 5, mRNA; gi|289546580|ref|NM_001172568.1| SEQ ID NO:8 Homo sapiens myeloid differentiation primary response 88 (MYD88), transcript variant 3, mRNA.

Polynucleotide sequences of target RNA sequences of mammalian and non-mammalian genes are available, for example, on the NCBI web site [http://www.ncbi.nlm.nih.gov/].

The sense strand and antisense strand of each double stranded molecule were chemically synthesized and chemically modified nucleotide monomers were incorporated into the strands. The chemical modifications utilized herein were as follows:

The sense strand and antisense strand were chemically synthesized and chemically modified nucleotide monomers were incorporated into the strands. The chemical modifications utilized herein were as follows:

Rac1_28:
SENSE STRAND
(SEQ ID NO: 9)
(5′ > 3′) CGUGCAAAGUGGUAUCCUG
and
ANTISENSE STRAND
(SEQ ID NO: 10)
(5′ > 3′) CAGGAUACCACUUUGCACG
Hes5_8:
SENSE STRAND
(SEQ ID NO: 11)
(5′ > 3′) GGGUUCUAUGAUAUUUGUA
and
ANTISENSE STRAND
(SEQ ID NO: 12)
(5′ > 3′) UACAAAUAUCAUAGAACCC
HEY2_8:
SENSE STRAND
(SEQ ID NO: 13)
(5′ > 3′) GGGUAAAGGCUACUUUGAU
and
ANTISENSE STRAND
(SEQ ID NO: 14)
(5′ > 3′) AUCAAAGUAGCCUUUACCC
MYD88_11:
SENSE STRAND
(SEQ ID NO: 15)
(5′ > 3′) GAAUGUGACUUCCAGACCA
and
ANTISENSE STRAND
(SEQ ID NO: 16)
(5′ > 3′) UGGUCUGGAAGUCACAUUC.

It is to be emphasized that the compounds (sequences and modifications) used herein are provided as examples only and should not, in any way, be considered as limiting the scope of the present invention.

### Example 2

### Synthesis of Sphingolipid-Spermine/Sphingolipid-Spermidine Phosphoramidite and siRNA

The synthesis of a sphingosine-spermine-phosphoramidite or sphingosine-spermidine-phosphoramidite and methods of generating sphingolipid-polyalkylamine oligonucleotides are disclosed in U.S. Patent Application Ser. No. 61/860,274 co-owned by applicants of the present application and co-filed with the present application, and incorporated by reference herein in its entirety. For example, a sphingolipid-polyalkylamine oligonucleotide compound may be synthesized using a sphingolipid-polyalkylamine phosphoramidite coupled to the 5′ terminus of a nucleotide in a synthesizer, for example, at the final step of synthesis. Alternatively, a sphingolipid-polyalkylamine compound may be coupled to a solid support followed by the addition of nucleotides to form a conjugate with a 2′ or 3′ linkage (sphingolipid-polyalkylamine covalently linked to the 2′ or 3′ position in the sugar of the terminal nucleotide of the oligonucleotide). Another possibility is to prepare the oligonucleotide and then, in a post synthesis step, to attach or couple the sphingolipid-polyalkylamine conjugate to a terminal nucleotide or internal nucleotide, after removal of a suitable protective group on the selected nucleotide, to form a linkage at a terminal site or at an internal site on the oligonucleotide. Preferably, the sphingolipid-polyalkylamine conjugate is attached to a terminal nucleotide, to form a conjugate with a linkage at a terminal site. For siRNA oligonucleotides, the sphingolipid-polyalkylamine conjugate may be attached to one terminus or both termini of the sense strand or to the 3′ terminus of the antisense strand, either directly or via a linker. The compounds generated by synthetic coupling or post-synthetic coupling are known as “conjugates”.

### Example 3

### In-Vitro Knockdown Activity of Sphingolipid Spermine siRNA Compounds

Chemically synthesized RAC1, HES5, HEY2 and MYD88 compounds linked or unlinked to a sphingolipid-polyalkylamine moiety (Table 1) were tested for knockdown activity of target mRNA. Gene target knockdown activity was studied using the psiCHECK™ system (Promega), which enables the evaluation of the intrinsic potency of inhibitory oligonucleotides, e.g. siRNA or antisense, by monitoring the changes in the activity of a Luciferase reporter gene carrying the target sites in its 3′ untranslated region (3′-UTR). The activity of a siRNA (unconjugated or conjugated to a sphingolipid polyalkylamine) toward the target sequence results in, for example, cleavage and subsequent degradation of the fused mRNA or in translation inhibition of the encoded protein. In addition, the psiCHECK™-2 vector contains a second reporter gene, Firefly luciferase, transcribed from a different promoter and non-affected by the oligonucleotide under study, useful for normalization of Renilla luciferase expression across different transfections.

TABLE 1
Exemplary siRAC1 (siRNA targeting RAC1) strands and compounds
synthesized:

| | Sense strand | Antisense strand |
|---|---|---|
| Compound name | (5′ > 3′) | (5′ > 3′) |
| RAC1_28_S2045 | zSLSp; mC; rG; mU; | mU; rA; rG; rG; |
| | rG; mC; rA; rA; | rA; mU; rA; rC; |
| | rA; rG; mU; rG; | mC; rA; mC; rU; |
| | rG; mU; rA; rU; | mU; rU; mG; rC; |
| | mC; rC; mU; Ra | mA; rC; mG |
| RAC1_28_S2081 | zSLSpdp; mC; rG; mU; | mU; rA; rG; rG; |
| | rG; mC; rA; rA; | rA; mU; rA; rC; |
| | rA; rG; mU; rG; | mC; rA; mC; rU; |
| | rG; mU; rA; rU; | mU; rU; mG; rC; |
| | mC; rC; mU; rA | mA; rC; mG |
| RAC1_28_S2139 | zSLSpdp; mC; rG; mU; | rUps; rA; rG; rG; |
| | rG; mC; rA; rA; | rA; 2fU; rA; |
| | rA; rG; mU; rG; | 2fC; 2fC; 2fA; 2fC; |
| | rG; mU; rA; rU; | 2fU; 2fU; 2fU; |
| | mC; rC; mU; rA | rG; 2fC; rA; 2fC; |
| | | rGps; zdTps; zdT$ |
| RAC1_28_S1908 | mC; rG; mU; rG; | mU; rA; rG; rG; |
| | mC; rA; rA; rA; | rA; mU; rA; rC; |
| | rG; mU; rG; rG; | mC; rA; mC; rU; |
| | mU; rA; rU; mC; | mU; rU; mG; rC; |
| | rC; mU; rA | mA; rC; mG |

RAC1_28_S2045: (Sphingolipid-Spermine Conjugated siRNA to RAC1)

sense strand (SEQ ID NO:4) with 2′-O-methyl sugar modified ribonucleotides present in position (5′>3′) 1, 3, 5, 10, 13, 16 and 18, a sphingolipid-spermine moiety covalently attached to the 5′ terminus, and a 3′ phosphate.

antisense strand (SEQ ID NO:5) with 2′-O-methyl sugar modified ribonucleotides present in position (5′>3′) 1, 6, 9, 11, 13, 15, 17 and 19, and a 3′ phosphate.

RAC1_28_S2081: (Sphingolipid-Spermidine Conjugated siRNA to RAC1)

sense strand (SEQ ID NO:4) with 2′-O-methyl sugar modified ribonucleotides present in position (5′>3′) 1, 3, 5, 10, 13, 16 and 18, a sphingolipid-spermidine moiety covalently attached to the 5′ terminus, and a 3′ phosphate.

antisense strand (SEQ ID NO:5) with 2′-O-methyl sugar modified ribonucleotides present in position (5′>3′) 1, 6, 9, 11, 13, 15, 17 and 19, and a 3′ phosphate.

RAC1_28_S2139 (Sphingolipid-Spermidine Conjugated siRNA to RAC1)

sense strand (SEQ ID NO:3) with 2′-O-methyl sugar modified ribonucleotides present in position (5′>3′) 1, 3, 5, 10, 13, 16 and 18, a sphingolipid-spermidine moiety covalently attached to the 5′ terminus, and a 3′ phosphate.

antisense strand (SEQ ID NO:4) with 2′-deoxy-fluro sugar modified ribonucleotides present in position (5′>3′) 6, 8, 9, 10, 11, 12, 13, 14, 16 and 18, a dTdt overhang covalently attached to the 3′ terminus and phosphorothioate linkages between nucleotides 1-2, and between the 3′ terminal nucleotide and the dT and between dT-dT.

RAC1_28_S1908 (Unlinked Control Molecule):

sense strand (SEQ ID NO:4) with 2′-O-methyl sugar modified ribonucleotides present in position (5′>3′) 1, 3, 5, 10, 13, 16 and 18, and a 3′ phosphate.

antisense strand (SEQ ID NO:5) with 2′-O-methyl sugar modified ribonucleotides present in position (5′>3′) 1, 6, 9, 11, 13, 15, 17 and 19, and a 3′ phosphate.

Similar modifications were utilized to generate the HES5, HEY2 and MYD88 sphingolipid-polyalkylamine oligonucleotide compounds, set forth in Table 2.

TABLE 2
HES5, HEY2 and MYD88 sphingolipid-polyalkylamine oligonucleotide
compounds (siHES5, siHEY2, siMYD88).

| Compound name | Sense strand (5′ > 3′) | Antisense strand (5′ > 3′) |
|---|---|---|
| HES1_36_S2390 | zSLSp; mC; rA; rG; rC; rG; rA; rG; rU; rG; | 5′p; rA; rU; mC; rG; rU; rU; rC2p; rA; |
| | rC; rA; rU; rG; rA; rA2p; rC2p; rG2p; rA2p; | mU; rG; mC; rA; rC; rU; mC; rG; rC; mU; |
| | rU2p; zc3p | rG; zc3p; zc3p |
| HES5_8_S2323 | zc3p; rG; rG; rG; rU; rU; rC; mU; rA; mU; | 5′p; mU; rA; mC; rA; rA; rA; rU2p; rA; |
| | rG; rA; mU; rA; rU; rU; mU; rG; mU; rA; zc3p | rU; rC; rA; rU; mA; rG; rA; rA; rC; rC; rC |
| HES5_8_S2391 | zSLSp; rG; mG; rG; mU; rU; mC; rU; mA; | 5′p; mU; rA; mC; rA; rA; rA; mU; rA; rU; |
| | rU; mG; rA; mU; rA; mU; rU; mU; rG; mU; | rC; rA; rU; mA; rG; rA; rA; rC; rC; rC; |
| | rA; zc3p | zc3p; zc3p |
| HES5_8_S2392 | zSLSp; rG; rG; rG; rU; rU; rC; mU; rA; mU; | 5′p; mU; rA; mC; rA; rA; rA; mU; rA; rU; |
| | rG; rA; mU; rA; rU; rU; mU; rG; mU; rA; | rC; rA; rU; mA; rG; rA; rA; rC; rC; rC; |
| | zc3p | zc3p; zc3p |
| HES5_8_S2395 | zSLSp; rG; mG; rG; mU; rU; mC; rU; mA; | mU; rA; mC; rA; rA; rA; mU; rA; rU; rC; |
| | rU; mG; rA; mU; rA; mU; rU; mU; rG; mU; | rA; rU; mA; rG; rA; rA; rC; rC; rC; zc3p; |
| | rA; zc3p | zc3p |
| HES5_8_S2396 | zSLSp; rG; rG; rG; rU; rU; rC; mU; rA; mU; | mU; rA; mC; rA; rA; rA; mU; rA; rU; rC; |
| | rG; rA; mU; rA; rU; rU; mU; rG; mU; rA; | rA; rU; mA; rG; rA; rA; rC; rC; rC; zc3p; |
| | zc3p | zc3p |
| HEY2_2_S2399 | zSLSp; rG; rG; rG; mU; rA; rA; rA; rG; rG; | mU; rU; mC; rA; rA; rA; rG2p; mU; rA; |
| | rC; mU; rA; mC; rU; rU; mU; rG; rA; rA; | rG; mC; rC; mU; rU; mU; rA; mC; rC; |
| | zc3p | mC; zc3p; zc3p |
| HEY2_8_S2402 | zSLSp; rG; rG; rG; mU; rA; rA; rA; rG; rG; | 5′p; rA; rU; mC; rA; rA; rA; rG2p; mU; |
| | rC; mU; rA; mC; rU; rU; mU; rG; rA; rU; | rA; rG; mC; rC; mU; rU; mU; rA; mC; rC; |
| | zc3p | mC; zc3p; zc3p |
| HEY2_8_S2410 | zSLSp; rG; rG; rG; mU; rA; rA; rA; rG; rG; | rA; rU; mC; rA; rA; rA; rG2p; mU; rA; rG; |
| | rC; mU; rA; mC; rU; rU; mU; rG; rA; rU; | mC; rC; mU; rU; mU; rA; mC; rC; mC; |
| | zc3p | zc3p; zc3p |
| MYD88_11_S2289 | zidB; rG; rA; rA; rU; rG; rU; rG; rA; rC; rU; | 5′p; mU; rG; rG; mU; mC; mU; rG; rG; |
| | rU; rC; rC; rA; rG2p; rA2p; rC2p; rC2p; rA2p; | mA; rA; rG; mU; mC; rA; mC; rA; mU; |
| | zc3p | mU; mC; zc3p; zc3p$ |
| MYD88_11_S2327 | zSLSp; rG; rA; rA; rU; rG; rU; rG; rA; rC; | 5′p; mU; rG; rG; mU; mC; mU; rG; rG; |
| | rU; rU; rC; rC; rA; rG2p; rA2p; rC2p; rC2p; | mA; rA; rG; mU; mC; rA; mC; rA; mU; |
| | rA2p | mU; mC; zc3p; zc3p$ |
| MYD88_11_S2477 | zidB; rG; rA; rA; rU; rG; rU; rG; rA; rC; rU; | 5′p; mU; rG; rG; mU; mC; mU; rG2p; rG; |
| | rU; rC; rC; rA; rG2p; rA2p; rC2p; rC2p; rA2p; | mA; rA; rG; mU; mC; rA; mC; rA; mU; |
| | zc3p | mU; mC; zc3p; zc3p |
| MYD88_11_S2479 | zSLSp; rG; rA; rA; rU; rG; rU; rG; rA; rC; | 5′p; mU; rG; rG; mU; mC; mU; rG2p; rG; |
| | rU; rU; rC; rC; rA; rG2p; rA2p; rC2p; rC2p; | mA; rA; rG; mU; mC; rA; mC; rA; mU; |
| | rA2p | mU; mC; zc3p; zc3p |
| MYD88_11_S2478 | zidB; rG; rA; rA; rU; rG; rU; rG; rA; rC; rU; | 5′p; mU; rG; rG; mU; mC; rU2p; rG; rG; |
| | rU; rC; rC; rA; rG2p; rA2p; rC2p; rC2p; rA2p; | mA; rA; rG; mU; mC; rA; mC; rA; mU; |
| | zc3p | mU; mC; zc3p; zc3p |
| MYD88_11_S2480 | zSLSp; rG; rA; rA; rU; rG; rU; rG; rA; rC; | 5′p; mU; rG; rG; mU; mC; rU2p; rG; rG; |
| | rU; rU; rC; rC; rA; rG2p; rA2p; rC2p; rC2p; | mA; rA; rG; mU; mC; rA; mC; rA; mU; |
| | rA2p | mU; mC; zc3p; zc3p |
| MYD88_11_S2509 | zc3p; rG; rA; rA; rU; rG; rU; rG; rA; rC; rU; | 5′p; mU; rG; rG; mU; mC; mU; rG2p; rG; |
| | rU; rC; rC; rA; rG2p; rA2p; rC2p; rC2p; rA2p; | mA; rA; rG; mU; mC; rA; mC; rA; mU; |
| | zc3p | mU; mC; zc3p; zc3p |
| MYD88_11_S2511 | zSLSp; rG; rA; rA; rU; rG; rU; rG; rA; rC; | 5′p; mU; rG; rG; mU; mC; mU; rG2p; rG; |
| | rU; rU; rC; rC; rA; rG2p; rA2p; rC2p; rC2p; | mA; rA; rG; mU; mC; rA; mC; rA; mU; |
| | rA2p; zc3p | mU; mC; zc3p; zc3p |
| MYD88_11_S2510 | zc3p; rG; rA; rA; rU; rG; rU; rG; rA; rC; rU; | 5′p; mU; rG; rG; mU; mC; rU2p; rG; rG; |
| | rU; rC; rC; rA; rG2p; rA2p; rC2p; rC2p; rA2p; | mA; rA; rG; mU; mC; rA; mC; rA; mU; |
| | zc3p | mU; mC; zc3p; zc3p |
| MYD88_11_S2512 | zSLSp; rG; rA; rA; rU; rG; rU; rG; rA; rC; | 5′p; mU; rG; rG; mU; mC; rU2p; rG; rG; |
| | rU; rU; rC; rC; rA; rG2p; rA2p; rC2p; rC2p; | mA; rA; rG; mU; mC; rA; mC; rA; mU; |
| | rA2p; zc3p | mU; mC; zc3p; zc3p |

TABLE 3
Legend for compound Tables 1 and 2

| Modification Code | Modification Description |
|---|---|
| $ | No 3′ Phosphate |
| m | 2′-O-methyl ribo-nucleotide-3′-phosphate |
| Ld | Spiegelmer deoxy-nucleotide (mirror image DNA) |
| lna | Locked deoxy Nucleic Acid (deoxy) |
| psiU | PseudoUridine |
| rN2p | ribo-nucleotide-2′-phosphate |
| nc | Nicked |
| zdT | Deoxy-Thymidine-3′-Phosphate |
| zidT | Inverted-Deoxy-Thymidine-5′-Phosphate |
| zcy3 | Cyanine Dye (Red Excitation) |
| 3mN2p | 3′-O-methyl ribo-nucleotide-2′-phosphate |
| mNpeth | 2′-O-methylnucleotide-3′-ethoxyphosphate |
| d | deoxyribose-5′-phosphate |
| zdT; zdT | overhang at 3′ |
| zidB | Inverted abasic deoxyribose-5′-phosphate; At 5′ = 5′-5′ idAb; |
| | At 3′ = 3′-3′ idAb |
| ziLd | Inverted L-DNA |
| zc6Np | Amino-C6-Phosphate |
| 5′p | 5′-regular Phosphate |
| dB | abasic deoxyribose-3′-phosphate (Tetrahydrofuran) |
| Lr | mirror image RNA |
| zc12Np | Amino-C12-Phosphate |
| zOle | Oleic acid |
| zPalm; zc6Np | Palmitoyl-Amino-C6-Phosphate |
| z(c6Np)2-SD | (C6-Amino-Pi)2-Symmetrical Doubler |
| zc5Np | Amino-C5-Phosphate |
| m5r | 5-Methyl-ribonucleotide (cytidine/uridine) |
| zrA; zrG | rArG |
| zirB | Inverted abasic ribose-5′-phosphate |
| zrB; zrB | abasic ribose-3′-phosphate x2 |
| zirB; zirB | Inverted abasic ribose-5′-phosphate x2 |
| zdB; zdB | abasic deoxyribose-3′-phosphate x2 |
| zc3p; zc3p | 1,3-Propanediol-Pi x2 = (CH2)3-Pi x2 |
| zc3p; zrG | (CH2)3-Pi_rG |
| zc3p; zrB | (CH2)3-Pi; ribo-Abasic-3′-Pi |
| zc3p | (CH2)3-Pi = 3-Hydroxypropane-1-phosphate |
| z(c12Np)2-SD | (C12-Amino-Pi)2-Symmetrical Doubler |
| z(c12p)2-SD | (C12-Pi)2-Symmetrical Doubler |
| zpRNH3 | Amino Modifier Serinol (Glen Research) |
| zpRNH3; zpRNH3 | Amino Modifier Serinol x 2 (Glen Research) |
| dC(C6N) | Amino-Modifier-C6-dC (dC-derivative) |
| zdC(C6N) | Amino-Modifier-C6-dC (dC-derivative) |
| zdC(C6N); zdC(C6N) | Amino-Modifier-C6-dC x2 (dC-derivative) |
| dT(C2N) | Amino-Modifier-C2-dT (dU-derivative) |
| dC(N4al) | deoxy Cytidine N4 Amino linker |
| zdC(N4al) | deoxy Cytidine N4 Amino linker |
| zpRNH3; zpRNH3; zpRNH3 | Amino Modifier Serinol x 3 (Glen Research) |
| zc6Np; zrC; zrA | Amino-C6-Phosphate_rCrA |
| d | deoxyUridine |
| rNps | Phosphorothioated RNA base (rNps = rN*) |
| zc3p; zc3p; zc3p | (CH2)3-Pi x3; = 3-Hydroxypropane-1-phosphate; |
| zc3p; zc3ps | (CH2)3-pi_1,3-Propanediol-Phosphorotioate |
| zb | 1,3-bis(hydroxymethyl)benzene |
| zb; zb | 1,3-bis(hydroxymethyl)benzene X2 |
| zc3p; zcy3 | (CH2)3-Pi (=3-Hydroxypropane-1-phosphate); Cyanine Dye |
| zmU | 2′-O-methyluridine-3′-ethoxyphosphate |
| zmC | 2′-O-methylCytidine-3′-phosphate |
| zLdT | L-deoxyriboThymidine-3′-phosphate |
| idB | Inverted abasic deoxyribose-5′-phosphate |
| s | 5′ phosphorothioate = non-cleavable Pi |
| zLdA | L-deoxyriboAdenosine-3′-phosphate |
| zLdC | L-deoxyriboCytidine-3′-phosphate |
| yLdG | replaced by L-deoxyriboGuanosine-3′-phosphate |
| dNps | Phosphorothioated DNA base (dNps = dN*) |
| zdU | cap deoxyUridine |
| zc3p; zc3p; zcy3 | (CH2)3-Pi; (CH2)3-Pi; Cyanine Dye |
| zc6Np; zc6p | NH2-C6-pi_(CH2)6-pi |
| zc6Np; zc12p | NH2-C6-pi_(CH2)12-pi |
| zLdG | L-deoxyriboGuanosine-3′-phosphate |
| ptd | Pyrazolo-triazine Deoxy, C—C nucleoside |
| zc12Np | Amino-C12-Phosphate |
| z(CH2CH2O)3p; z(CH2CH2O)3p | (CH2CH2O)3-pi_(CH2CH2O)3-pi |
| zTHNBc6p; zc6p | Tetrahydronaphtalene-butyric-C6 phosphate_(CH2)6-pi |
| zTHNBc6p; z(CH2CH2O)3p | Tetrahydronaphtalene-butyric-C6-phosphate_(CH2CH2O)3- |
| | pi |
| zmG | 2′-O-methylGuanosine-3′-phosphate; |
| zGlu | Glutamic acid |
| z4FB-CONH2; zc12Np | 4-Formylbenzoate Sodium_C12-Amino-Pi |
| zHS; zc6p | mercapto radical (HS)_(CH2)6-pi |
| zSLSp | SphingoLipid-Spermine_phosphate |
| zTHNBc6p | Tetrahydronaphtalene-butyric-C6 phosphate |
| zSLSp; zThiC6SSp | SphingoLipid-Spermine-pi_Thiol Modifier-C6—S—S-phosphate |
| zSLSpd; zThiC6SSp | SphingoLipid-Spermidine-pi_Thiol Modifier-C6—S—S- |
| | phosphate |
| zSLSpdp | SphingoLipid-Spermidine-phosphate |
| zThiC6SSp | Thiol Modifier-C6—S—S-phosphate |
| zc6Np; zThiC6SSp | NH2-C6-pi_Thiol Modifier-C6—S—S-phosphate |
| ztnaA | TNA adenosine |
| ztnaC | TNA cytidine |
| dtna | D-Threose Nucleic Acid |
| mNps | Phosphorothioated-2′OMe RNA base (mNps = mN*) |
| 2f | 2′-deoxy-2′-fluoro nucleoside |
| zdTps; zdT | Thymidine-Phosphorothioate; Thymidine_overhang at 3′end |
| zThiC6SSp; zVEp | Thiol Modifier-C6—S—S_Vitamin E-pi |
| zPGA; zc6Np | PGA_NH2-C6-pi |
| ptr | Pyrazolo-triazine Ribo, C—C nucleoside |
| zPGA;;c6Np; zThiC6SSp | PGA_NH2-C6-pi_Thiol Modifier-C6—S—S-phosphate |
| rN2ps | Ribo-nucleotide-2′-phosphorotioate; Phosphorothioated 2′-5′- |
| | bridge (rN2ps = rN2*) |
| zc3ps; zc3p | 1,3-Propanediol-Phosphorotioate_(CH2)3 |
| zc3ps | 1,3-Propanediol-Phosphorotioate |
| zSD | Symmetrical Doubler |
| z(VEp)2-SD | (Vitamin E-Pi)2-Symmetrical Doubler |
| zptrA | rA-Pyrazolo-triazine |
| zptrA; zptrA | rA-Pyrazolo-triazine x2 |
| zc3ps; zc3ps | 1,3-Propanediol-Phosphorotioate x2 |
| zptdA | dA-Pyrazolo-triazine |
| zptdA; zptdA | dA-Pyrazolo-triazine x2 |
| zbAla | b-Alanine (beta amino acid) |

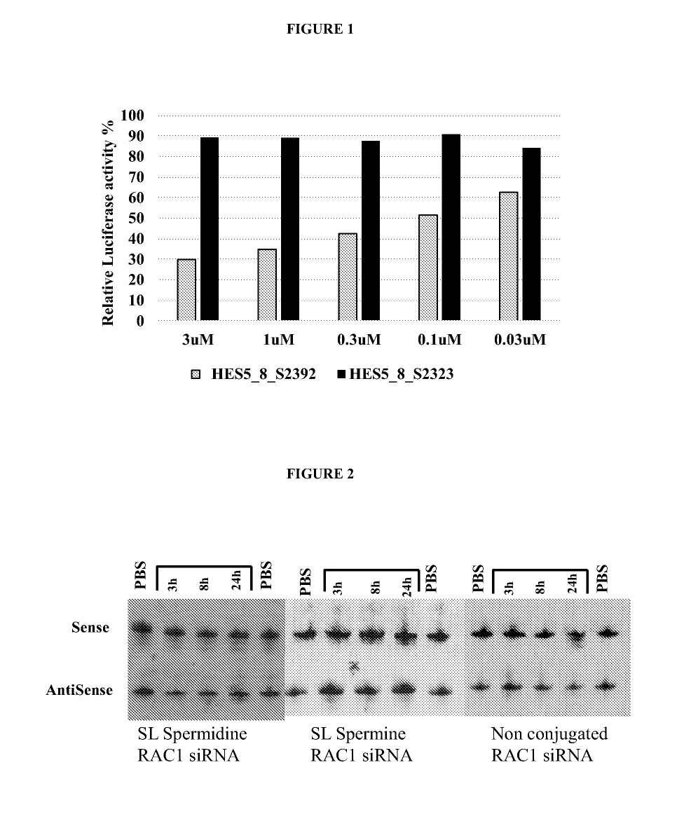

A psiCHECK™-2-based construct was prepared for the evaluation of the on-target activity of the guide strands (GS) of Rac1, HES1, HEY2 and MYD88 sphingolipid polyalkylamine siRNA compounds. In the construct, one copy of the full target sequence of the GS was cloned into the multiple cloning site located in the 3′-UTR of the Renilla luciferase, downstream to the stop codon. The psiCHECK™-2 plasmid was transfected into human HeLa cells. The transfected HeLa cells were then seeded into a 96-well plate and incubated at 37° C. with the siRNA of interest added in duplicates and without transfection reagent. The final siRNA concentrations of the sphingolipid polyalkylamine siRNA compounds tested were 0.03, 0.1, 0.3, 1, and 3 μM. Control cells were not exposed to any siRNA. 48 hours following siRNA addition, the cells were harvested for protein extraction. Renilla and FireFly Luciferase activities were measured in individual cell protein extracts using the Dual-Luciferase® Assay kit according to the manufacturer's procedure. Renilla Luciferase activity values were normalized by Firefly Luciferase activity values obtained from the same samples. siRNA activity was expressed as percentage of residual normalized Renilla Luciferase activity in a test sample from the normalized Renilla Luciferase activity in the control cells. FIG. 1 is a graph showing dose-dependent, transfection reagent-independent knockdown of Renilla Luciferase activity buy a sphingolipid-spermine conjugated siHES5 compound (HES5_8_S2392) compared to an unconjugated siHES5 compound (HES5_8_S2323).

The stability of the chemically synthesized and modified siRNAs either non-conjugated or conjugated to sphingolipid (SL) spermine (Table 1) against degradation by nucleases was analyzed. Stability of the siRNAs was analyzed in both rabbit vitreous.

The siRNA compounds were incubated for 24 hours at 37° C. in either or rabbit vitreous. At time points between 0 and 24 hours after incubation, 1 ng aliquots were transferred to TBE-loading buffer, snap frozen in liquid nitrogen and stored at −20° C. until use. The aliquots were thawed on ice and analyzed by non-denaturing polyacrylamide gel electrophoresis. Based on the gel migration patterns, presented in FIG. 2, the sphingolipid polyalkylamine siRNA compounds target Rac1 were found to be stable for at least 24 hours at 37° C. in rabbit vitreous. Similar results were obtained for the HES5 and HEY32 conjugates in vitreous fluid and in cerebrospinal fluid (CSF) data not shown.

### Example 4

### Sphingolipid Spermine or Sphingolipid Spermidine Oligonucleotides Display Improved Accumulation and Prolonged Residence Time in the Retina Upon Intravitreal Injection

4A) In the present experiment, the level of unconjugated, sphingolipid spermine or sphingolipid spermidine siRAC1 compounds (Table 1, above, RAC1_28_S1908, RAC1_28_S2045, RAC1_28_S2081) was determined in the rat retina 24 hours after IVT injection.

Each experimental group included 6 independently injected eyes of adult male Sprague Dawley rats (8-12 week old) into which 20 ug (microgram) siRNA/10 μl PBS were injected. At 24 hours post IVT injection, rats were euthanized, eyes—harvested, retinas—dissected and subjected to siRNA quantification by Stem and Loop (S&L) qPCR method. Total RNA was prepared from retina samples using EZ-RNA II Total RNA Isolation Kit (Biological Industries, #20-410-100). In some cases, triton extracts were prepared from the retina samples: retina samples were weighed and a 10× volume of 0.25% preheated Triton X-100 was added to each sample. The mixtures were vortexed, incubated at 95° C. for 10 min, cooled on ice (10 min) and finally centrifuged (20,000 g, 20 min, 4° C.). Supernatants were collected.

For specific amplification of the siRNA contained in the total RNA samples (or in the triton extracts), complementary DNA (cDNA) was prepared by a reverse transcription (RT) reaction using Superscript II kit (Invitrogen, #18064-014), 1 μg total RNA (or 5 μl triton extract supernatant) as template and a Stem and Loop (S&L) primer, which is partially complementary to the antisense strand of the subject siRNA and in addition, harbors a stem and loop structure at its 5′-end.

RT primers: For RAC1-28 amplification. 1648-2/Rac128ASRT:

(SEQ ID NO: 17)
GTCGTATCCAGTGCAGGGTCCGAGGTATTCGCACTGGATACGACCGTG
CAA

The resulting cDNA served as a template for siRNA amplification using the SYBER-Green based quantitative PCR (qPCR) method (SYBR Green Master Mix, Applied Biosystems; #4309155) and two amplification primers: one complementary to the siRNA sequence and the second complementary to the stem and loop region of the RT primer.

qPCR primers: RAC1-28

1695-3/Rac128ASF2
(SEQ ID NO: 18)
CGGCGGCAGGATACCACTTTG
1681-1/Rev_3
(SEQ ID NO: 19)
AGTGCAGGGTCCGAGGTATT

For absolute quantification of siRNA in the test samples, standard curves were generated by the spiking of several known siRNA quantities (10-3 pmols) into retina extracts followed by RNA extraction and cDNA preparation as described above. Serial dilutions prepared from the spiked samples cDNA, were amplifies by qPCR. The resulting Ct values (Ct=Threshold Cycle, the PCR cycle in which fluorescence level exceeds a chosen threshold limit) obtained in each reaction, were plotted against the corresponding (Log₁₀) siRNA quantity values for the generation of a standard curve, which was then used for the quantification of siRNA in unknown samples by interpolation.

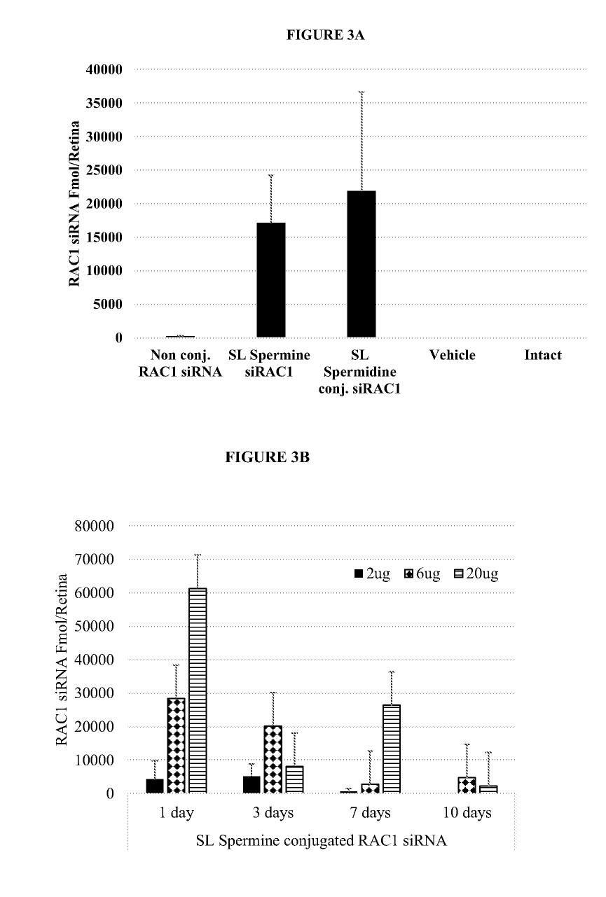

Results: FIG. 3A shows the siRNA concentration (fmole/retina) detected in rat retina 24 hours after IVT injection. As can be seen, while non-conjugated siRNA was detected in the retinal tissues 24 hours post IVT injection in low levels, siRNA compounds lined to sphingolipid spermine and sphingolipid spermidine were detected in amounts approximately 100 times higher than the non-conjugated control compounds.

4B) In the present experiment, the delivery of sphingolipid (SL) spermine siRAC1 compound was assessed. The presence of said siRNA compound in the retina of the treated rats was analyzed 1, 3 and 7 days post IVT injection. Each experimental group included 6 independently injected eyes of adult male Sprague Dawley rats (8-12 week old) into which either 20 ug, 6 ug or 2 ug siRNA/10 μl PBS were injected. 1, 3 or 7 days post IVT injection, rats were euthanized, eyes were harvested, and the retinas dissected and subjected to siRNA quantification by Stem and Loop qPCR method (methods are described above in 4A). Rats injected with vehicle only (10 μl PBS) served as controls.

Results: the results of this study are summarized in FIG. 3B. Generally, as can be clearly seen in FIG. 3B, the accumulation of the sphingolipid spermine siRAC1 compound in the retinal tissue following IVT injection was significantly and substantially increased and in most cases showed dosage dependent accumulation in the retina. Additionally, the sphingolipid spermine siRAC1 compound displayed prolonged residence time in the retinal tissue and was detected in the retina at least 10 days post IVT injection.

### Example 5

### Retinal Distribution of Sphingolipid Polyalkylamine siRNA Compounds Following Intravitreal Injection in Rats

Retinal distribution of chemically synthesized and modified RAC1 siRNAs (listed in Table 1) either non-conjugated or sphingolipid spermine- or sphingolipid spermidine siRNA compounds (RAC1_28_S2045, RAC1_28_52081) were analyzed by siRNA in situ hybridization (siISH) 24 hours post intravitreal injection of 20 ug of each of the siRNAs into rat eyes. Two independent studies (4 eyes for each compound) were performed.

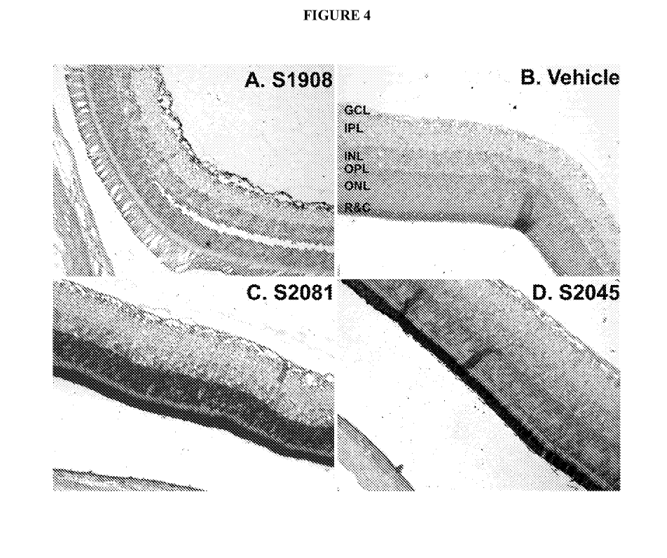

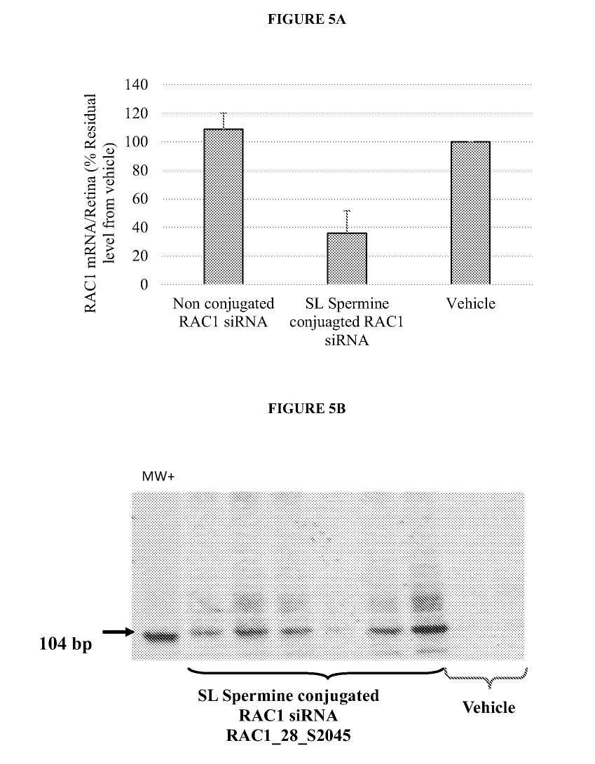

Enucleated eyes were immersed in 10% neutral buffered formalin (NBF) followed by paraffin embedding and sectioning. All samples were subjected to extensive sectioning (250 micron intervals) and several sets of about 8 representative sections (representing approximately half of the eye) were collected from each eye sample. Sections were mounted on slides and subjected to siISH with an oligonucleotide probe complementary to the antisense strand of the siRAC1_28 used in these studies. The probes were labeled with digoxygenin The sections were subjected to microscopic examination by a skilled histopathologist masked to the study group identity. Eye structures and retinal layers displaying detectable siRNA hybridization signals were recorded for each analyzed eye. The results of the analysis of the first study are summarized in Table 4 below. Representative siISH images of retinal sections are shown in FIG. 4.

Both types of sphingolipid-polyalkylamine siRNA compounds and non-conjugated siRNA displayed hybridization signals in the retinal layers proximal to the vitreous (the injection site), including the ganglion cell layer (GCL) and nerve fiber layer (NFL), although intensity of the hybridization signals obtained with non-conjugated siRNA was much weaker than those obtained with both types of the sphingolipid-polyalkylamine siRNA compounds. The non-conjugated siRNA was not detected in the retinal layers more distal to the vitreous, while both sphingolipid-polyalkylamine siRNA compounds displayed very prominent hybridization signals in the retinal layers more distal to the vitreous. Specifically, very strong hybridization signals were observed in, in the outer nuclear layer (ONL) in the majority of the eyes injected with both sphingolipid polyalkylamine siRNA compounds, as well as in the inner nuclear layer (INL) of some of the injected eyes. Moreover, very prominent hybridization signals associated with both sphingolipid-polyalkylamine siRNA compounds were detected in the layer of rods and cones (R&C) and in the retinal pigment epithelium (RPE).

TABLE 4
In situ hybridization results of Study 1:

| | siRNA hybridization signals in the eye, N (siRNA-positive eyes)/(4 eyes examined) | | | | | | | | | |
|---|---|---|---|---|---|---|---|---|---|---|
| siRNA | RPE | R&C | ONL | OPL | INL | IPL | GCL | NFL | Iris | Lens |
| RAC1_28_S1908 | 1 | 2 | 0 | 0 | 0 | 0 | 1 | 1 | 1 | 2 |
| RAC1_28_S2081 | 4 | 4 | 4 | 1 | 1 | 1 | 4 | 4 | 4 | 4 |
| RAC1_28_S2045 | 3 | 3 | 3 | 0 | 0 | 0 | 0 | 3 | 0 | 3 |
| Vehicle | 0 | 0 | 0 | 0 | 0 | 0 | 0 | 0 | 0 | 0 |

The results of the in situ siRNA hybridization analysis of the second study (Table 5) further confirmed retinal distribution of sphingolipid-polyalkylamine conjugated siRNA compounds observed in the first study. Prominent siRNA hybridization signals were observed in all retinal layers examined, in most of the eyes injected with both sphingolipid-polyalkylamine conjugated siRNAs. Negative siRNA signals were observed in vehicle-treated eyes (not shown).

TABLE 5
In situ hybridization results of sphingolipid-polyalkylamine siRNA
compound-injected eyes of Study 2.

siRNA hybridization signals
in the eye, N (siRNA-positive
eyes)/(4 eyes examined)

| siRNA | R&C | ONL | INL | GCL |
|---|---|---|---|---|
| RAC1_28_S2081 | 3 | 4 | 2 | 4 |
| RAC1_28_S2045 | 4 | 4 | 2 | 3 |

These experiments demonstrate that sphingolipid-polyalkylamine siRNA compounds (both spermine and spermidine-types) administered to the eye by intravitreal injection were taken up and widely distributed in all retinal layers (both proximal and distal to the vitreous)—GCL, INL, ONL, R&C and RPE. Distribution to GCL, ONL and RPE was the most consistently observed.

FIG. 4 shows retinal distribution patterns of non-conjugated and two types of sphingolipid-polyalkylamine conjugated RAC1-28 siRNAs, 24 hours after the IVT injection into the rat eye (original magnification ×200). Control eyes were injected with the vehicle

### Example 6

### Effects of Sphingolipid-Spermine, Sphingolipid-Spermidine or Non Conjugated RAC1 Compounds Upon Intravitreal Administration in Rats

6A) In the present study the target knockdown efficiency, confirmation of RNAi mechanism of action and analysis of potential pro-inflammatory effects of sphingolipid spermine conjugated and non conjugated compounds were assessed in rat retina 24 hours after intravitreal injection. In the present experiment a dose of 20 μg of the RAC1 siRNA (RAC1_28_S1908 and RAC1_28_S2045), in 10 μL of PBS vehicle was microinjected into the vitreous body of adult, Sprague-Dawley rats (6 eyes per experimental group). A control group was injected in the same manner with PBS vehicle. Study was terminated 24 hours after siRNA/vehicle administration. Animals were euthanized, eyes harvested, retinas dissected and subjected to RNA extraction. RNA from each sample was used to quantify RAC1 mRNA levels by qPCR (knockdown assessment). For quantifying the Rac1 mRNA levels, total RNA was prepared from retina samples using EZ-RNA II Total RNA Isolation Kit (Biological Industries, #20-410-100). Complementary DNA (cDNA) was prepared using Superscript II kit (Invitrogen, #18064-014)) from 1 μg RNA in a 15 μl reaction. The resulting cDNA served as a template for specific transcript qPCR based amplification using SYBR Green Master Mix (Applied Biosystems; #4309155) and target gene specific amplification primers.

For the determination of target gene transcript amount in each sample, a standard DNA fragment was prepared from the target gene amplicon (the target gene region amplified by qPCR). A series of qPCR reactions was performed on several known amounts (10 pg-100 attogr) of the standard DNA. A standard curve was then generated by plotting the Ct values (threshold cycle—the number of cycles that were needed for the fluorescence to exceed a chosen threshold) obtained in the qPCR reactions against the corresponding (Log₁₀) standard quantity values. The amount of target transcript/s in the experimental samples was determined by interpolation to the standard curve. The amounts of the target gene transcript were normalized against the amounts of at least two reference gene transcripts, βActin and PPIA

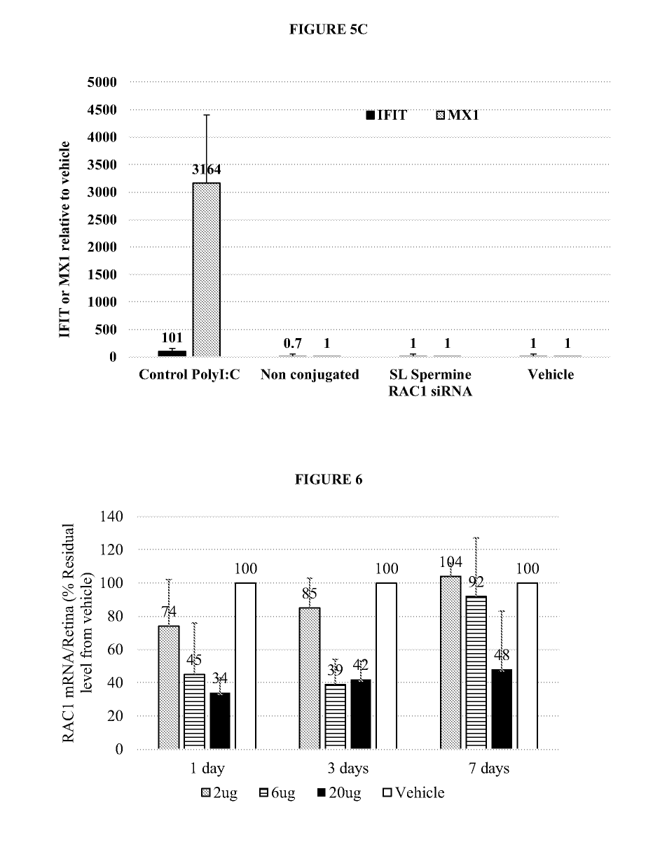

The results are presented in FIG. 5A as an average of the mRNA quantity per retina (presented as % of residual levels of intact eyes) obtained for each group (N=6 eyes). As can be seen in FIG. 5A, only sphingolipid spermine RAC1 siRNA elicited RAC1 mRNA knockdown activity.

The RNAi-mediated cleavage of RAC1 mRNA in the rat eye following IVT administration of the sphingolipid spermine siRNA compound was confirmed by Rapid Amplification of cDNA Ends (RACE). RNAi-mediated cleavage of a target mRNA occurs between nucleotides complementary to bases 10-11 of the siRNA guide strand to produce two mRNA fragments: a 5′ fragment representing the region upstream to the cleavage site and the 3′-fragment representing the region downstream to the cleavage site. The presence of the downstream fragment can be detected using the RACE method, which is based on the ligation of an oligonucleotide adapter to the 5′ end of this fragment, followed by RT-PCR amplification using adapter-specific forward and gene-specific reverse primers. RACE analysis is based on ligation of the extracted RNA with a RAC1 siRNA specific RNA adaptor oligonucleotide

Ligation mix (10-20 ul) included Ligase Buffer, 2 u/ul T4 RNA Ligase (New England Biolabs), 1nMATP and 4 u/ul RnaseOut (Invitrogen #10777019). Ligation mix was incubated at 37° C. for 1 hour followed by RNA precipitation under 70% ethanol and suspension in 5-10 ul distilled water. Ligated RNA (up to 5 ul) was subjected to reverse transcription (RT) by a target gene specific primer using 200 Unit/ul SuperScriptIII RT (Invitrogen #18080-093). RT reaction (10 ul) was incubated at 50° C. for 60 min and terminated by heating at 70° for 15 min Transcribed cDNA (5 ul of RT reaction) was amplified by PCR using an adaptor specific primer GenRace_F3 (5′-CGACTGGAGCACGAGGACACTGCAT) (SEQ ID NO:20) together with one gene specific primer (PCR program: Annealing 600C, elongation time—30 sec, ×15 cycles). Products (10 ul) of the PCR reaction (1^{st} PCR) were used for further PCR amplification (2^{nd} PCR, 100 ul reaction volume) with an adaptor specific primer GenRace_F4 (5′-GGACACTGCATGGACTGAAGGAGTA) (SEQ ID NO:21) together with a second (nested) gene specific primer (PCR program: Annealing 60° C., elongation time—30 sec, ×30 cycles)

The second PCR products (20 ul) were separated on 8% non-denaturing acrylamide gel (1×TBE, 90v, 36 mA, 50 min). Gels were stained by Ethidium Bromide and then heated at 95° C. in a water bath, in a sealed plastic bag for 10 min. DNA was then transferred to HybondN+ (Amersham) membrane in a semi dry blotter at 500 mA for 20 min Blots were pre-hybridization: in 6×SSC, 1×Denhardt, 0.5% SDS, 250 ug/ml SSDNA, 500 ug/ml tRNA, 0.05% NaPPi, at 420 C for ˜2 hours. A ³³P-end-labeled target specific oligonucleotide probe (1pmol/ml) was added to the hybridization mix. Hybridization was carried out by overnight at 42° C. Blots were washed of excess probe (two 40 min washes in 2×SSC+0.5% SDS at 42° C.) and then exposed to an X-ray film for 3-72 hrs. Ref52 rat cells transfected with 20 nM of RAC1 siRNA served as the positive control (produces a 104 bp band).

The results presented in FIG. 5B indicate the generation of the specific proper RT-PCR (RACE) product as predicted for RNAi-mediated cleavage of RAC1 mRNA by sphingolipid spermine siRAC1 compound.

The extent of the interferon (IFN) response following IVT injection of sphingolipid spermine siRNA compounds and non-conjugated siRNA molecules was further quantified by measuring mRNA levels of genes involved in the IFN response (MX1 and IFIT1) using the IFNr qRT-primers system. The levels of IFN-responsive genes were quantified using quantitative RT-PCR (described above) and expressed as the fold difference relative to levels measured in non-treated animals (FIG. 5C). As can be seen in FIG. 5C, IVT injection of PolyI:C (positive control) induced a strong increase in the expression levels of IFIT1 and MX1. Sphingolipid spermine siRAC1 (siRNA targeting RAC1) compounds did not induce the IFN-responsive genes in the eye.

6B) In the present study dosage dependent and duration of target knockdown efficiency, of sphingolipid spermine siRAC1 compounds (as in section 4A) were assessed in rat retina 1 day, 3 days and 7 days after intravitreal injection. siRNA was exemplified by siRNA targeting RAC1 mRNA and structural details of the compounds used are summarized in Table 1 above. In the present experiment a dose of 20 μg, 6 μg and 2 μg of the sphingolipid spermine siRAC1 in 10 μL of PBS vehicle was microinjected into the vitreous body of adult, Sprague-Dawley rats (6 eyes per experimental group). A control group was injected in the same manner with PBS vehicle. The activity of the siRNA compounds in the retina of the treated rats was analyzed 1, 3 and 7 days post IVT injection. 1, 3 or 7 days post IVT injection, rats were euthanized, eyes—harvested, retinas—dissected and subjected to RNA extraction. RNA from each sample was used to quantify RAC1 mRNA levels by qPCR (knockdown assessment). The results are presented in FIG. 5 as an average of the RAC1 mRNA quantity per inner ear (presented as % of residual levels of Vehicle ears) obtained for each group. As can be seen in FIG. 6, the sphingolipid spermine siRAC1 displayed significant knockdown activity with dosage dependent effect. Moreover the knockdown activity could be observed also after 7 days from treatment.

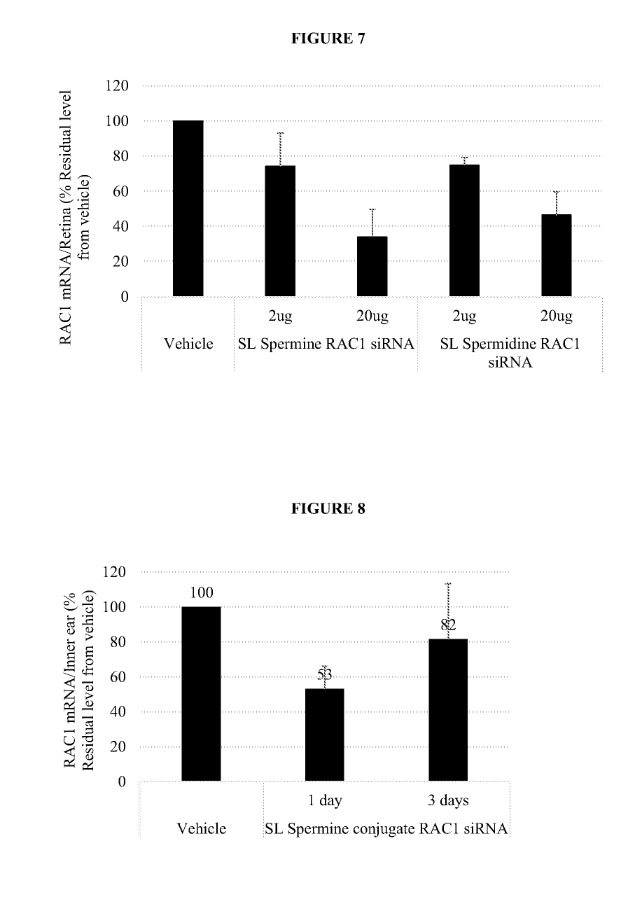

4C) In the present study target knockdown efficiency, of sphingolipid spermine and sphingolipid spermidine conjugated siRNA were assessed in rat retina 24 hours after intravitreal injection. siRNA was exemplified by siRNA targeting RAC1 (RAC1_28_S2045 and RAC1_28_S2081). In the present experiment a dose of 20 μg, and 2 μg of the RAC1 siRNA listed in table 1 above, in 10 μL of PBS vehicle was microinjected into the vitreous body of adult, Sprague-Dawley rats (6 eyes per experimental group). A control group was injected in the same manner with PBS vehicle. The activity of the siRNA compounds in the retina of the treated rats was analyzed 24 hours post IVT injection. Each experimental group included 6 independently injected eyes of adult male Sprague Dawley rats (8-12 week old) into which either Sphingolipid Spermine or Sphingolipid Spermidine at either 20 μg, or 2 μg siRNA/10 μl PBS were injected. 24 hours post IVT injection, rats were euthanized, eyes—harvested, retinas—dissected and subjected to RNA extraction. RNA from each sample was used to quantify RAC1 mRNA levels by qPCR (knockdown assessment). The results are presented in FIG. 7 as an average of the RAC1 mRNA quantity per inner ear (presented as % of residual levels of vehicle ears) obtained for each group. As can be seen in FIG. 7, both the sphingolipid spermine and sphingolipid spermidine siRNA compounds displayed significant and dosage dependent knockdown activity.

The extent of the interferon (IFN) response following IVT injection of sphingolipid spermine conjugated and non-conjugated siRNA molecules was further quantified by measuring mRNA levels of genes involved in the IFN response (MX1 and IFIT1) using the IFNr qRT-primers system. The levels of IFN-responsive genes were quantified using quantitative RT-PCR (described above) and expressed as the fold difference relative to levels measured in non-treated animals. IVT injection of PolyI:C (positive control) induced a strong increase in the expression levels of IFIT1 and MX1. Sphingolipid spermine or sphingolipid spermidine siRAC1 did not induce the IFN-responsive genes in the eye (data not shown).

### Example 7

### The Effects of Sphingolipid Spermine siRNA Compounds Targeting RAC1 mRNA in the Cochlea of Rats Upon Transtympanic Administration

In the present study, the target knockdown efficiency of sphingolipid spermine conjugated siRNA co were assessed in the rat cochlea 1 and 3 days after transtympanic injection. Each experimental group included 5 adult male Sprague Dawley rats (8-12 week old). siRNA was exemplified by siRNA targeting RAC1 mRNA (RAC1_28_S2045).

The siRNA compounds were administered by transtympanic injection into the left middle ear cavity, at 60 ug in 20 μl 0.5% hyaluronic Acid (HA). Rats administered with vehicle only (20 μl of 0.5% hyaluronic Acid (HA) and untreated rats (intact group) served as negative control. Animals were sacrificed 1 day and 3 days after siRNA administration. Soft cochlea tissues were dissected from bony cochlea of each animal and subjected to RNA extraction. RNA from each sample was used to quantify RAC1 mRNA levels by qPCR (knockdown assessment; methods are described above; example 4). The results are presented in FIG. 8 as an average of the RAC1 mRNA quantity per ug (microgram) RNA extracted from inner ear (presented as % of residual levels of intact ears) obtained for each group. As can be seen in FIG. 8, the sphingolipid spermine conjugated siRNA compound displayed significant knockdown activity already after 1 day from treatment, as indicated by the lower % residual levels of RAC1 in sphingolipid spermine conjugated siRNA. Significant although lower knockdown activity could still be observed after 3 days from treatment.

### Example 8

### Sphingolipid Spermine siRNA Compounds Display Improved Accumulation in the Lung Upon Intratracheal Administration Compared to Non Conjugated siRNA

In the present study, the delivery of sphingolipid spermine and non conjugated siRNA compounds were assessed in mice lungs 24 hours after intratracheally administered. Each experimental group included 6 adult C57BL/6 mice (10-12 week old). siRNA was exemplified by siRNA targeting RAC1 mRNA (RAC1_28_S1908 and RAC1_28_S2045)

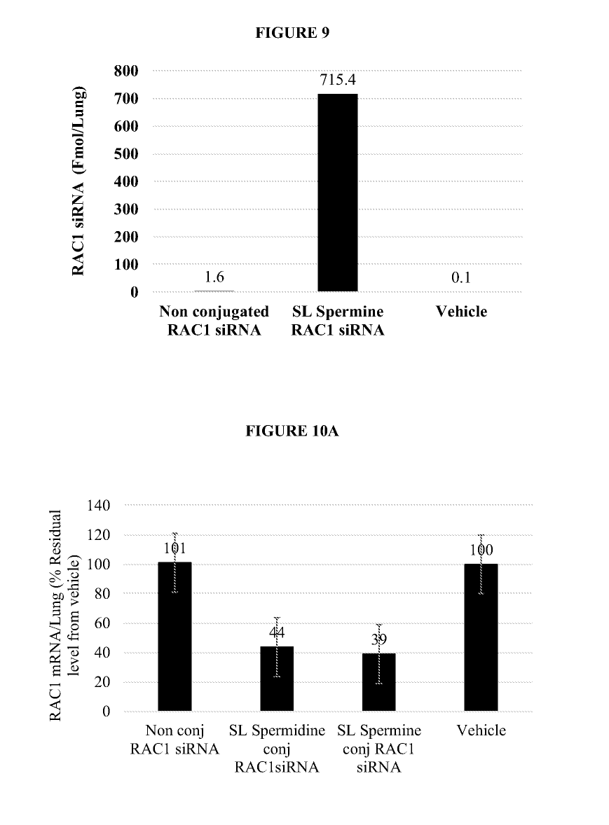

For administering intratracheally (I.T.), the trachea was exposed by blunt dissection, under the dissecting microscope. A sterile 27/30-gauge needle was used to tracheal puncture between the cartilage rings. The siRNA solution (50 μl) or saline (50 μl) was slowly injected by 0.3 ml syringe, with the needle tip directed towards the lungs. Control group was treated with sterile saline. Animals were sacrificed 24 hours after siRNA administration. Both left and right lungs were dissected from each animal and subjected to RNA extraction and subjected to siRNA quantification by Stem and Loop qPCR method (methods are described above in Example 4A). FIG. 9 shows the siRNA concentration (fmole/ug RNA) detected in mice lungs 24 hours after intratracheally administered. As can be seen, while non conjugated siRNA could hardly be detected in the lung tissues 24 hours post intratracheally administered injection, while sphingolipid spermine siRNA compounds were detected in amounts at least 400 times higher.

### Example 9

### The Effects of Sphingolipid Polyalkylamine siRNA Compounds or Non Conjugated siRNA Compounds Targeting RAC1 in the Lung Upon Intratracheal Administration

In the present study, target knockdown efficiency, confirmation of RNAi mechanism of action and analysis of potential pro-inflammatory effects of sphingolipid spermine siRNA compounds, and sphingolipid spermidine siRNA compounds were assessed in mice lungs 24 hours after intratracheally administered. Each experimental group included 6 adult C57BL/6 mice (10-12 week old). The oligonucleotides were exemplified by siRNA targeting RAC1 mRNA and structural details of the compounds (RAC1_28_S1908, RAC1_28_S2045, RAC1_28_S2081) used are summarized in Table 1 above.

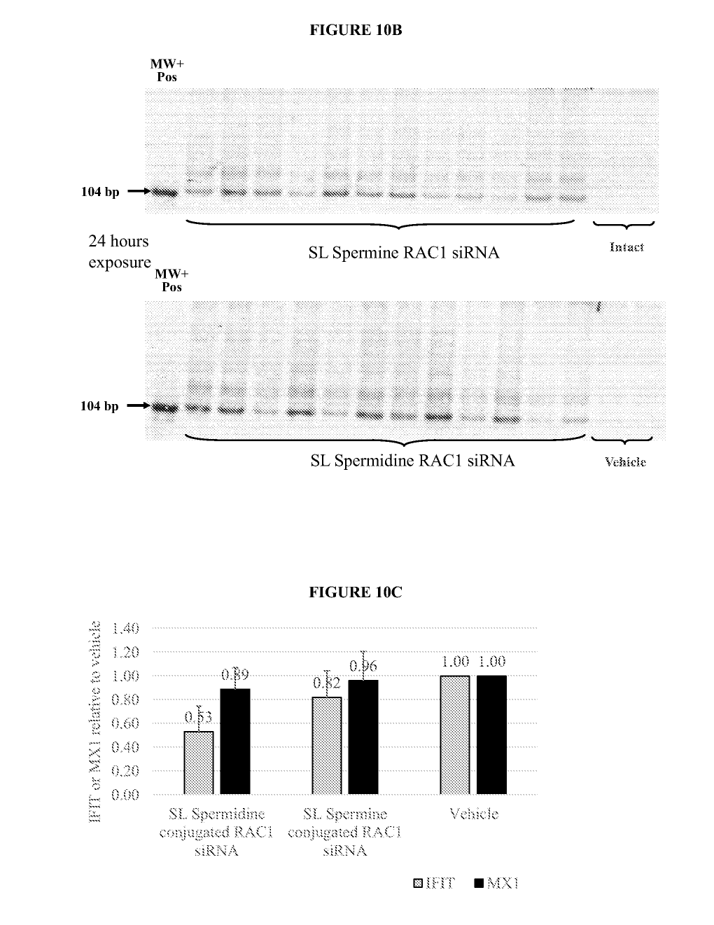

All siRNAs were administered intratracheally (I.T.). The trachea was exposed by blunt dissection, under the dissecting microscope. A sterile 27/30-gauge needle was used to tracheal puncture between the cartilage rings. The siRNA solution (50 μl) or saline (50 μl) was slowly injected by 0.3 ml syringe, with the needle tip directed towards the lungs. Control group was treated with sterile saline. Animals were sacrificed 24 hours after siRNA administration. Both left and right lungs were dissected from each animal and subjected to RNA extraction. RNA from each sample was used to quantify RAC1 mRNA levels by qPCR (knockdown assessment), for confirmation of the RNAi mechanism of action (RACE analysis) and for qPCR quantification of the expression levels of interferon-inducible genes as a measure of potential siRNA-elicited pro-inflammatory effects in the ear (all methods are described above; Example 6). The results of RAC1 mRNA levels are presented in FIG. 10A as an average of the RAC1 mRNA quantity per mg lung tissue (presented as % of residual levels in vehicle treated lungs) obtained for each group. As can be seen in FIG. 10A, the sphingolipid spermine and sphingolipid spermidine siRNA compounds displayed significant knockdown activity while no change in RAC1 mRNA levels was observed in the lung of mice treated with non conjugated siRNA. The RNAi-mediated cleavage of RAC1 mRNA in the lungs following intratracheal administration of the sphingolipid spermine and sphingolipid spermine siRNA compounds was confirmed by Rapid Amplification of cDNA Ends (RACE). RNAi-mediated cleavage of a target mRNA occurs between nucleotides complementary to bases 10-11 of the siRNA guide strand to produce two mRNA fragments: a 5′ fragment representing the region upstream to the cleavage site and the 3′-fragment representing the region downstream to the cleavage site. The presence of the downstream fragment can be detected using the RACE method, which is based on the ligation of an oligonucleotide adapter to the 5′ end of this fragment, followed by RT-PCR amplification using adapter-specific forward and gene-specific reverse primers. For RACE analysis is based on ligation of the RNA extracted with a RAC1 siRNA specific RNA adaptor oligonucleotide (detailed method described in Example 6).

Ref52 rat cells transfected with 20 nM of RAC1 siRNA served as the positive control (produces a 104 bp band).

The results presented in FIG. 10B indicate the generation of the specific RT-PCR (RACE) product predicted for RNAi-mediated cleavage of RAC1 mRNA by sphingolipid spermine or sphingolipid spermine siRNA compounds.

The extent of the interferon (IFN) response following IVT injection of sphingolipid spermine siRNA compounds and non-conjugated siRNA molecules was further quantified by measuring mRNA levels of genes involved in the IFN response (MX1 and IFIT1) using the IFNr qRT-primers system. The levels of IFN-responsive genes were quantified using quantitative RT-PCR (described above) and expressed as the fold difference relative to levels measured in non-treated animals (FIG. 10C). As can be seen in FIG. 10C, RAC1 sphingolipid spermine or sphingolipid spermidine siRNA compounds did not induce expression of the IFN-responsive genes in the eye.

### Example 10

### Rat Model of Aminoglycoside-Induced Hair Cell Loss

An ototoxic combination of kanamycin and ethacrynic acid is used for almost complete damage of the auditory sensory epithelia leading to a complete loss of auditory (hearing) function in Norway Brown rats. Assessment of hearing function is performed by monaural Auditory Brainstem Response (ABR) audiometry (a neurologic test of auditory brainstem function in response to auditory (click) stimuli of different frequencies), prior to (on day 0), and after (on day 3) the ototoxic damage, followed by fortnightly measurements till the end of the study. The animals are sacrificed 10 weeks after the study begins and dissected cochlea are processed for histology and immunohistochemical evaluation of the hair cell markers.

Study details: On day 0, kanamycin (KM, 200 mg/ml) and ethacrynic acid (EA, 20 mg/ml) cocktail (in PBS (pH 8.0) is injected transtympanically to Norway Brown rats. Loss of hearing function is confirmed by increased ABR threshold levels on day 3. On day 4, animals are randomized to two study groups, receiving a combination of non-conjugated or sphingolipid polyalkylamine siRNA compounds as described herein against, for example HES1, HES5 and/or HEY2 at a dosage of 30 ug of each siRNAs/ear or vehicle (sterile saline). The test siRNA compounds and vehicle are administered by application of a 3 ul volume on the GelFoam piece placed onto the round window membrane via surgical access.

Analysis of the results shows that the sphingolipid-polyalkylamine siRNA compounds cause regeneration of the auditory sensory epithelia and consequent restoration of auditory function in chemically—induced hearing loss in rats.

### Example 11

### Testing Potential Efficacy of Sphingolipid-Polyalkylamine Oligonucleotide Compounds in Restoration of Auditory Function in 120 dB Broad Band Noise-Induced Hearing Loss Model in Mice

Feasibility of hearing restoration and hair cell regeneration upon local application of application of a combination of non-conjugated or sphingolipid-polyalkylamine siRNA compound against, for example, HES1, HES5 and/or HEY2 (as described herein) is assessed in a mouse noise-induced hearing loss model. The model used is essentially similar to that described in Wang Y, et al., J Assoc. Res in Otolaryngology. 2002; 03:248-268, with the following modifications. Female FBV mice (Maison et al., J. Neurosci. 2002: 22(24): 10838-46) are subjected to acoustic trauma produced by a 2 hr exposure to an 8-16 kHz octave band noise presented at 120 dB SPL causing nearly complete loss of the hair cells and subsequent loss of hearing function. Upon confirmation of the hearing function loss (assessed by measurement of the ABR thresholds, as described in Example 10) early after the noise insult, the mice are treated with sphingolipid-polyalkylamine siRNA compounds or vehicle. The test compounds are introduced via direct intratympanic injection, in a 5 μl injection volume. Functional recovery is followed by fortnightly monaural ABR measurements until the end of the study. Tissue harvest and processing is carried out at termination. All ears from all animals are fixed for further cochlea dissection followed by histopathology and immunohistochemical evaluation of the hair cell markers.

Analysis of the results shows that the sphingolipid-polyalkylamine siRNA compounds cause regeneration of the auditory sensory epithelia and consequent restoration of auditory function in noise—induced hearing loss in mice.

### Example 12

### Testing Potential Efficacy of Sphingolipid-Polyalkylamine siRNA Compounds in Restoration of Auditory Function in Mouse Cre-loxP Conditional Gene Expression Model of Hearing Loss

A recently developed Cre-loxP technology for conditional gene expression in the inner ear of the mice (reviewed in Cox, et al., J Assoc Res Otolaryngol. 2012; 13(3): 295-322) is utilized in this study, to damage consistently the outer hair cells in the early postnatal mouse cochlea. Specifically, a mouse model is created by crossing the following two lines: (a) prestin-CreER transgenic mouse line (CreER allele where an altered ligand-binding domain of the estrogen receptor is fused to Cre which expression is controlled by an outer hair-cell—specific Prestin promoter) and (b) Rosa-DTA (diphtheria toxin) reporter mouse line. This cross produces heterozygous offspring where DTA may be induced by tamoxifen injection on specific postnatal days, causing death of the outer hair cells and complete loss of auditory function in these mice. Upon confirmation of the hearing function loss (assessed by measurement of the ABR thresholds, as described in Example 10) early after the hair cell death, the mice are treated with sphingolipid-polyalkylamine siRNA compounds or vehicle. The test compounds are introduced via direct intratympanic injection, as described in Example 11. Auditory function is followed by a fortnightly monaural ABR measurements until the end of the study. Tissue harvest and processing is carried out at termination. All ears from all animals are fixed for further cochlea dissection followed by histopathology and immunohistochemical evaluation of the hair cell markers. Morphological assessment is performed subsequently.

Analysis of the results shows that the sphingolipid-polyalkylamine siRNA compounds cause regeneration of the auditory sensory epithelia and consequent restoration of auditory function in genetically—induced hearing loss in mice.

Although the above examples have illustrated particular ways of carrying out embodiments of the invention, in practice persons skilled in the art will appreciate alternative ways of carrying out embodiments of the invention, which are not shown explicitly herein. It should be understood that the present disclosure is to be considered as an exemplification of the principles of this invention and is not intended to limit the invention to the embodiments illustrated.

Those skilled in the art will recognize, or be able to ascertain using no more than routine experimentation, equivalents of the specific embodiments of the invention described herein. Such equivalents are intended to be encompassed by the following claims.

1. A sphingolipid-polyalkylamine-oligonucleotide compound having general formula I: 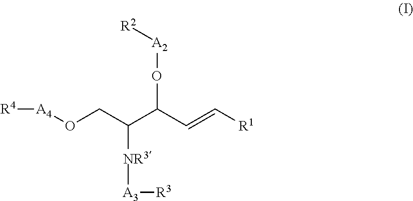
wherein
R¹ is a branched or linear C₇-C₂₄ alkyl, alkenyl or polyenyl;
R², R³ and R⁴ each independently is hydrogen, C₁-C₄ alkyl, a branched or linear polyalkylamine or derivative thereof, or an oligonucleotide;
R^{3′} is hydrogen or C₁-C₄ alkyl;
A₂, A₃ and A₄ each independently is present or absent but if present is one of C(O), C(O)NHX, C(O)NHR⁵X, C(O)R⁵X, C(O)R⁵C(O)X, R⁵X or R⁵OC(O)X;
R⁵ is a branched or linear C₁-C₂₀ alkyl chain optionally substituted with one or more heteroatoms;
X is present or absent but if present is S, P, O or NH;
at least one of R², R³ or R⁴ is a branched or linear polyalkylamine or derivative thereof;
at least one of R², R³ or R⁴ is an oligonucleotide;
or a salt of such compound;
for use in treating or preventing a disease or disorder in a subject; and
with the proviso that the disease or disorder is other than cancer.

2. The compound of claim 1, wherein the disease or condition is selected from the group consisting of an inner ear disease or disorder, an eye disease or disorder, a respiratory disease or disorder, a central nervous system or peripheral nervous system disease or disorder, a skin disease or disorder, a renal disease or disorder, a cardiac disease or disorder, a liver disease or disorder, inflammatory disease or disorder, an infectious (viral, bacterial, fungal) disease and a fibrotic disease or disorder of any organ.

3. The compound of claim 2, wherein the inner ear disease or condition is a hearing loss or a balance loss or disorder, and wherein the compound or salt of such compound is to be administered to the subject via a local otic administration route selected from ear drops to the tympanic membrane, transtympanic delivery to the middle ear and intraoperational delivery methods to the round window or to the endolymph.

4. The compound of claim 2, wherein the disease or condition is an eye disease or disorder, and wherein the compound or salt of such compound is to be administered to the subject via a local ocular administration route or local otic administration route selected from eye drops, intravitreal, subretinal, subscleral or transtympanic administration.

5. The compound of claim 2, wherein the disease or condition is a respiratory disease or disorder and wherein the compound or salt of such compound is to be administered to the subject via a local pulmonary administration routes selected from intranasal, intratracheal and/or intrabronchal instillation or inhalation.

6. The compound of claim 2, wherein the disease or condition is selected from a central nervous system disease or disorder or peripheral nervous system disease or disorder; and wherein the compound or salt of such compound is to be administered to the subject via a local administration route selected from intraotic, intrathecal/suprathecal or intraventricular administration.

7. The compound of claim 2, wherein the disease or condition is a skin disease or disorder; and wherein the compound or salt of such compound is to be administered to the subject via a local administration route selected from topical, subcutaneous or intradermal administration.

8. The compound of claim 2, wherein the disease or condition is selected from the group consisting of a cardiac disease or disorder; and wherein the compound or salt of such compound is to be administered to the subject via a local administration route selected from intramyocardial or intracoronary administration.

9. The compound of claim 2, wherein the disease or condition is a renal disease or disorder, a cardiac disease or disorder, a liver disease or disorder and an inflammatory or fibrotic disease or disorder of any location; and wherein the compound or salt of such compound is to be administered to the subject via parenteral administration selected from the group consisting of intravenous, intraarterial, subcutaneous, transdermal, intraperitoneal, and intramuscular administration.

10. The compound of any of claims 1 to 9, wherein in the sphingolipid-polyalkylamine oligonucleotide compound R¹ is C₇-C₂₄ alkyl selected from the group consisting of C₁₀-C₂₀ alkyl, C₁₀-C₁₆ alkyl and C₁₃ alkyl, preferably C₁₃ alkyl.

11. The compound of claim 10, wherein in the sphingolipid-polyalkylamine-oligonucleotide compound R¹ is C₁₃ alkyl, A₂ is C(O), A₃ is absent and R² is selected from spermine and having general formula (Ia) or spermidine and having general formula (Ib) 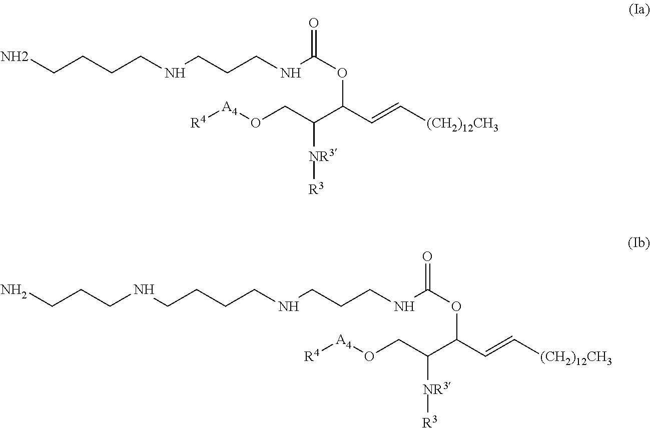
wherein
A₄ is selected from the group consisting of C(O), C(O)NHX, C(O)NHR⁵X, C(O)R⁵X, C(O)R⁵C(O)X, R⁵X and R⁵OC(O)X;
R⁵ is a branched or linear C₁-C₂₀ alkyl chain optionally substituted with one or more heteroatoms;
R³ and R^{3′} each independently is hydrogen or C₁-C₄ alkyl;
R⁴ is an oligonucleotide;
or a salt of such compound.

12. The compound of claim 11, wherein the oligonucleotide is a single-stranded oligonucleotide or a double-stranded oligonucleotide.

13. The compound of claim 12, wherein the single-stranded oligonucleotide is selected from an antisense nucleic acid (NA) molecule, pre-mRNA, a non-coding RNA, an aRNA, an aptamer, a ribozyme, a synthetic mRNA and shRNA.

14. The compound of claim 12, wherein the double-stranded oligonucleotide is a double stranded NA (dsNA) molecule capable of acting via RNA interference and selected from the group consisting of a siRNA, a miRNA and a miRNA mimetic.

15. The compound of any of claims 12 to 14, wherein the oligonucleotide is partially or fully chemically modified.

16. The compound of any of claims 1 to 15, wherein the double-stranded oligonucleotide has the duplex structure set forth below
5′ (N)x-Z 3′ (antisense strand)
3′ Z′—(N′)y-z″ 5′ (sense strand)
wherein each of N and N′ is an unmodified ribonucleotide, a modified ribonucleotide or an unconventional moiety;
wherein each of (N)x and (N′)y is an oligonucleotide in which each consecutive N or N′ is joined to the next N or N′ by a covalent bond;
wherein each of x and y is independently an integer from 8 to 49 and y is an integer from 15 to 49;
wherein z″ is present or absent, but if present is a capping moiety covalently attached to the 5′ terminus of the sense strand;
wherein each of Z and Z′ is independently present or absent, but if present is 1-5 consecutive nucleotides or non-nucleotide moieties or a combination thereof covalently attached at the 3′ terminus of the strand in which it is present;
wherein a sphingolipid-polyalkylamine is covalently attached to at least one of the 3′ terminus of the antisense strand, the 3′ terminus of the sense strand, the 5′ terminus of the sense strand or the 5′ terminus of the antisense strand;
wherein the nucleotide sequence of (N′)y has complete or partial complementarity to the nucleotide sequence of (N)x; and wherein (N)x comprises a nucleotide sequence with complete or partial complementary to a consecutive sequence in a target RNA;
wherein when the sphingolipid-polyalkylamine is attached at the 5′ terminus of the sense strand then z″ is absent;
wherein when the sphingolipid-polyalkylamine is attached at the 3′ terminus of the sense strand then Z′ is absent;
wherein when the sphingolipid-polyalkylamine is attached at the 3′ terminus of the antisense strand then Z is absent.

17. The compound of claim 16, wherein each covalent bond joining each consecutive N or N′ is independently selected from the group consisting of a phosphodiester bond, a phosphotriester bond and a phosphorothioate bond.

18. The compound of claim 16 or 17, wherein x is an integer from 19 to 25 and y is an integer from 15 to 25; preferably x=y=19.

19. The compound of any of claims 16 to 18, wherein a capping moiety (z″) is covalently attached to the 5′ terminus of (N′)y and is selected from the group consisting of an abasic ribose, an abasic deoxyribose; an inverted abasic ribose, an inverted abasic deoxyribose; C6-amino-Pi and a mirror nucleotide.

20. The compound of any of claims 16 to 19, wherein each of Z and Z′ is a non-nucleotide moiety independently selected from the group consisting of C3OH, C3Pi, C3Ps, C3Pi-C3OH, C3Pi-C3Pi, C3Ps-C3OH and C3Ps-C3Ps.

21. The compound of any of claims 14 to 20, wherein the 5′ terminal nucleotide of the antisense strand [(N)x] is a complementary DNA or is mismatched to the target RNA and is selected from the group of A, U, dA, dU, or dT with or without additional chemical modifications to the sugar and/or to the linkage, and wherein the corresponding nucleotide on the sense strand [(N′)y] is complementary to the 5′ terminal nucleotide of the antisense strand.

22. The compound of any of claims 16 to 21, wherein the sphingolipid-polyalkylamine is covalently attached to at least one of the 3′ terminus of the antisense strand, the 3′ terminus of the sense strand or the 5′ terminus of the sense strand.

23. The compound of any of claims 16 to 22, wherein at least one of N or N′ is a modified ribonucleotide or an unconventional moiety

24. The compound of claim 23, wherein the at least one unconventional nucleotide is present in (N′)y or (N)x and is selected from the group consisting of a 2′-5′ linked nucleotide, a threose nucleic acid (TNA), a locked nucleic acid (LNA), a pyrazolotriazine nucleotide and a mirror nucleotide.

25. The compound of any of claims 16 to 24, wherein x=y=19 and at least one of a 2′-5′ linked nucleotide is present in positions (5′>3′) 16, 17, 18, and 19 or 15, 16, 17, 18, and 19 of (N′)y and/or wherein a 2′-5′ linked nucleotide, a threose nucleic acid (TNA) or a mirror nucleotide is present in at least one of positions 6, 7, or 8 in (N)x.

26. The compound of any of claims 16 to 25, wherein at least one of N or N′ is a sugar modified ribonucleotide, wherein the sugar modification comprises a 2′ sugar modification selected from the group consisting of 2′O-methyl sugar modification (2′OMe), 2′deoxyribose sugar modification (2′H), 2′deoxyfluoro sugar modification (2′F), 2′-O-methoxyethyl (2′MOE) sugar modification and a 2′-amino sugar modification, preferably a 2′O-methyl sugar modification.

27. The compound of any of claims 16 to 26, wherein the target RNA is the transcription product of mammalian, viral, plant, fungal or bacterial genome representing either coding or non-coding RNA.

28. The compound of any of claims 1 to 27, for generating a plant with an altered phenotype or treatment a plant disease.

29. A sphingolipid-polyalkylamine siRNA compound selected from a compound set forth in Table 2.
